(19) United States
(12) Patent Application Publication
(10) Pub. No.: US 2025/0311689 A1
(43) Pub. Date: Oct. 9, 2025

(54) SEEDLING GERMINATION AND GROWTH CONDITIONS

(71) Applicant: PIONEER HI-BRED INTERNATIONAL, INC., JOHNSTON, IA (US)

(72) Inventors: AJITH ANAND, DAVIS, CA (US); PING CHE, AMES, IA (US); WILLIAM JAMES GORDON-KAMM, URBANDALE, IA (US); TODD J JONES, JOHNSTON, IA (US); LARISA A RYAN, ANKENY, IA (US); NAGESH SARDESAI, JOHNSTON, IA (US); NING WANG, JOHNSTON, IA (US); XINLI EMILY WU, JOHNSTON, IA (US)

(73) Assignee: PIONEER HI-BRED INTERNATIONAL, INC., JOHNSTON, IA (US)

(21) Appl. No.: 18/696,217

(22) PCT Filed: Sep. 26, 2022
(86) PCT No.: PCT/US2022/077036

§ 371 Date: Mar. 27, 2024

(51) Int. Cl. A01H 3/04

(52) U.S. Cl. CPC A01H 3/04

(57) ABSTRACT

Methods for pre-treating and transforming monocot leaf explants are provided.

### CROSS-REFERENCE TO RELATED APPLICATIONS

This application claims priority to International (PCT) Patent Application Serial No. PCT/US22/77036, filed on Sep. 26, 2022, which claims priority to and the benefit of U.S. Provisional Patent Application No. 63/249,191 filed on Sep. 28, 2021, all of which is hereby incorporated herein in its entirety by reference.

### REFERENCE TO SEQUENCE LISTING SUBMITTED ELECTRONICALLY

The official copy of the sequence listing is submitted electronically via Patent Center as an XML formatted sequence listing with a file named 8529.xml created on Sep. 22, 2022 and having a size of 3,828,345 bytes and is filed concurrently with the specification. The sequence listing comprised in this XML formatted document is part of the specification and is herein incorporated by reference in its entirety.

The sequence descriptions and sequence listing attached hereto comply with the rules governing nucleotide and amino acid sequence disclosures in patent applications as set forth in 37 C.F.R. §§ 1.831-1.835.

### FIELD OF THE DISCLOSURE

The present disclosure relates to the field of plant molecular biology, including genetic manipulation of plants. More particularly, the present disclosure pertains to the transformation of monocot leaf explants.

### BACKGROUND OF THE DISCLOSURE

In recent years, there has been a tremendous expansion of the capabilities for the genetic engineering of plants. Current transformation technology provides an opportunity to produce commercially viable transgenic plants, enabling the creation of new plant varieties containing desirable traits. One limitation of the genetic engineering of plants is the availability of plant tissue explants that are amenable to transformation since many plant tissue explants are recalcitrant to transformation and regeneration. Thus, there is a need for plant transformation methods permitting a broader range of transformable and regenerable plant explant tissues.

### SUMMARY OF THE DISCLOSURE

The present disclosure comprises methods and compositions for the pre-treatment of seedlings to produce leaf explants that exhibit improved transformation frequencies. In an aspect, a method of pre-treating a seedling with at least one chemical compound, the method comprising contacting the seedling with the at least one chemical compound and isolating leaf explants from the seedling for transformation, wherein the transformation frequency of the leaf explants from the pre-treated seedling is higher than the transformation frequency of leaf explants from seedlings not treated with the at least one chemical compound is provided.

In another aspect, the at least one chemical compound is an auxin selected from the group consisting of 2,4-D, dicamba, 2,4,5-T, 1-Naphthaleneacetic acid (NAA), picloram, indole-3-acetic acid, 4-chloroindole-3-acetic acid, phenylacetic acid, indole-3-butyric acid, and indole-3-propionic acid.

In another aspect, the at least one chemical compound is anti-oxidant selected from the group consisting of dithiolthreitol, cysteine, and glutathione.

In another aspect, the at least one chemical compound is gibberellic acid inhibitors selected from the group consisting of ancymidol, paclobutrazol, uniconazole, chlormequat chloride, mepiquat, AMO-1618 [(2′-isopropyl-4′-(trimethylammoniumchloride)-5′-methylphenylpiperidinecarboxylate)], clorphonium-Cl, tetcylacis, flurprimidol, inabenfide, pro-hexadione, trinexapac-ethyl, daminozide, exo-16,17-acetate, malic hydrazide, phoshor D, alar, chlorocholine chloride, cycocel, the fungicides tebuconazole and metconazole, trinexapac-ethyl, prohexadione-calcium, and exo-16,17-dihydro-GA5-13-acetate. In a further aspect, the gibberellic acid inhibitor is ancymidol. In yet a further aspect, the seedling is contacted with ancymidol at a concentration of about 1 mg/l to about 2 mg/l.

In still another aspect, the at least one chemical compound is an inhibitor of abscisic acid biosynthesis selected from the group consisting of norflurazon, fluridone, diflufenican, abamine, and nordihydroguaiaretic acid (NDGA).

In another aspect, the at least one chemical compound is an abscisic acid antagonist selected from the group consisting of AA1 [1-[(4-benzylpiperazin-1-yl)methyl]-2-sulfanylidene-1H,2H,5H,6H,7H,8H-[1,3,4]thiadiazolo[3,2-a][1,3]diazepin-5-one], AS6 [3′-hexylsulfanyl-ABA], DFPM ([5-(3,4-Dichlorophenyl) furan-2-yl]-piperidin-1-ylmethanethione), and RK460.

In another aspect, the at least one chemical compound is the abscisic acid agonist, pyrabactin.

In another aspect, the at least one chemical compound is a cytokinin selected from the group consisting of BAP [6-benylamino purine], BA [benzyladenine], kinetin, TDZ [thidiazuron], and trans-zeatin.

In another aspect, the at least one chemical compound is an anti-cytokinin selected from the group consisting of S-4893 [a 4-phenylquinazoline derivative], 4-phenylquinazoline, 6-(2,5-Dihydroxybenzylamino) purine (LGR-991), and 2-hydroxy-3-methyl-benzyladenine.

In another aspect, the at least one chemical compound is the cyclin-dependent kinase (CDK) inhibitor, caffeine.

In another aspect, the at least one chemical compound is an auxin agonist selected from the group consisting of quinclorac, RubNeddins1 (RN1), RubNeddins2 (RN2), RubNeddins3 (RN3), and RubNeddins4 (RN4).

In another aspect, the at least one chemical compound is an auxin antagonist selected from the group consisting of p-Chlorophenoxy isobutylic (PCIB), tert-butoxycarbonylaminohexyl-IAA (BH-Indole Acetic Acid=BH-IAA), α-(phenylethyl-2-oxo)-IAA (PEO-IAA), and α-(2,4-dimethylphenylethyl-2-oxo)-IAA (auxinole).

In another aspect, the at least one chemical compound is a methylation inhibitor selected from the group consisting of 5-azacytidine, Decitabine, RG108 [N-Phthalyl-L-tryptophan], zebularine, thioguanine, 2′-deoxy-5-fluoreocytidine, CM-272 (6-methoxy-2-(5-methyl-2-furyl)-N-(1-methyl-4-piperidyl)-7-(3-pyrrolidin-1-ylpropoxy) quinolin-4-amine), CM-579 (6-methoxy-2-(5-methyl-2-furyl)-N-[(1-methyl-4-piperidyl)methyl]-7-(3-pyrrolidin-1-ylpropoxy) quinolin-4-amine), SGI-1027 [N-[4-[(2-Amino-6-methyl-4-pyrimidinyl)amino]phenyl]-4-(4-quinolinylamino)benzamide], gamma-oryzanol, and (−)-epigallocatechin gallate (ECGC), β-Thujaplicin, and procainamide.

In another aspect, the at least one chemical compound is a histone deacetylase (HDAC) inhibitor selected from the group consisting of valproic acid, tacedinaline, cambinol, trichostain A (TSA), verinostat, panobinostat, belinostat, RGFP-966 [(E)-N-(2-amino-4-fluorophenyl)-3-(1-cinnamyl-1H-pyrazol-4-yl) acrylamide], tubastatin A, and PCI-34051 [N-Hydroxy-1-[(4-methoxyphenyl)methyl]-1H-indole-6-carboxamide].

In another aspect, the at least one chemical compound is a sugar composition selected from the group consisting of sucrose and maltose.

In another aspect, the at least one chemical compound is a composition comprising a metal selected from the group consisting of copper, zinc, and manganese.

In another aspect, the at least one chemical compound is an amino acid selected from the group consisting of proline, asparagine, methionine, and glutamine.

In another aspect, the at least one chemical compound is an ethylene inhibitor selected from the group consisting of silver nitrate, 2-aminoethoxyvinyl glycine (AVG), silver ions (Ag), and 1-methylcyclopropene (1-MCP).

In another aspect, the at least one chemical compound is the ethylene competitive inhibitor, norbornadiene.

In another aspect, the at least one chemical compound is a polycomb repressive complex 2 (PRC2) inhibitor selected from the group consisting of EPZ005687 [1-cyclopentyl-N-[(4,6-dimethyl-2-oxo-1H-pyridin-3-yl)methyl]-6-[4-(morpholin-4-ylmethyl)phenyl]indazole-4-carboxamide], UNC1999 [N-[(6-methyl-2-oxo-4-propyl-1H-pyridin-3-yl)methyl]-1-propan-2-yl-6-[6-(4-propan-2-ylpiperazin-1-yl) pyridin-3-yl]indazole-4-carboxamide], PF-06726304 [C₂₂H₂₁C₁₂N₃O₃], Lirametostat, Tazemetostat, EPZ011989 (C₃₅H₅₁N₅O₄·HCl), CPI-169 (C₂₇H₃₆N₄O₅S), and JQ-EZ-05 (C₃₀H₃₈N₈O₂).

In another aspect, the at least one chemical compound is a Jasmonate, selected from the group consisting of Jasmonic acid and Methyl-jasmonate.

In another aspect, the at least one chemical compound is a safener selected from the group consisting of naphthalic anhydride, salicylic acid, fenchlorazole, cloquintocet-mexyl, and mefenpyr diethyl.

In another aspect, the at least one chemical compound is a kinase inhibitor selected from the group consisting of N-[(2R)-2,3 dihydroxypropoxy]-3,4-difluoro-2-(2-fluoro-4-iodoanilino) benzamide, anthra (1,9-cd) pyrazol-6 (2H)-one:4-(4-Fluoro phenyl)-2-(4-methylsulfinylphenyl)-5-(4-pyridyl) 1H imidazole, and N-benzyl-2-(pyrimidin-4-ylamino)-1,3 thiazole-4-carboxamide.

In another aspect, the at least one chemical compound is a reactive oxygen species (ROS) scavenger selected from the group consisting of Tiron, dimethylthiourea (DMTU), and diphenyleneiodonium (DPI).

In a further aspect, the at least one chemical compound is 2,4-D, dicamba, 2,4,5-T, NAA, picloram, indole-3-acetic acid, 4-chloroindole-3-acetic acid, phenylacetic acid, indole-3-butyric acid, indole-3-propionic acid, dithiolthreitol (DTT), cysteine, glutathione, ancymidol, paclobutrazol, uniconazole, chlormequat chloride, mepiquat, AMO-1618 [(2′-isopropyl-4′-(trimethylammoniumchloride)-5′-methylphenylpiperidinecarboxylate)], clorphonium-Cl, tetcylacis, flurprimidol, inabenfide, pro-hexadione, trinexapac-ethyl, daminozide, exo-16,17-acetate, malic hydrazide, phoshor D, alar, chlorocholine chloride, cycocel, the fungicides tebuconazole and metconazole, trinexapac-ethyl, prohexadione-calcium, exo-16,17-dihydro-GA5-13-acetate, norflurazon, fluridone, diflufenican, abamine, nordihydroguaiaretic acid (NDGA), AA1 [1-[(4-benzylpiperazin-1-yl)methyl]-2-sulfanylidene-1H,2H,5H,6H,7H,8H-[1,3,4]thiadiazolo[3,2-a][1,3]diazepin-5-one], AS6 [3′-hexylsulfanyl-ABA], DFPM ([5-(3,4-Dichlorophenyl) furan-2-yl]-piperidin-1-ylmethanethione), RK460, pyrabactin, BAP [6-benylamino purine], BA [benzyladenine], kinetin, TDZ [thidiazuron], trans-zeatin, S-4893 [a 4-phenylquinazoline derivative], 4-phenylquinazoline, 6-(2,5-Dihydroxybenzylamino) purine (LGR-991), 2-hydroxy-3-Methyl-benzyladenine, caffeine, quinclorac, RubNeddins1 (RN1), RubNeddins2 (RN2), RubNeddins3 (RN3), RubNeddins4 (RN4), p-Chlorophenoxy isobutylic (PCIB), tert-butoxycarbonylaminohexyl-IAA (BH-Indole Acetic Acid=BH-IAA), α-(phenylethyl-2-oxo)-IAA (PEO-IAA), α-(2,4-dimethylphenylethyl-2-oxo)-IAA (auxinole), 5-azacytidine, Decitabine, RG108 [N-Phthalyl-L-tryptophan], zebularine, thioguanine, 2′-deoxy-5-fluoreocytidine, CM-272 (6-methoxy-2-(5-methyl-2-furyl)-N-(1-methyl-4-piperidyl)-7-(3-pyrrolidin-1-ylpropoxy) quinolin-4-amine), CM-579 (6-methoxy-2-(5-methyl-2-furyl)-N-[(1-methyl-4-piperidyl)methyl]-7-(3-pyrrolidin-1-ylpropoxy) quinolin-4-amine), SGI-1027 [N-[4-[(2-Amino-6-methyl-4-pyrimidinyl)amino]phenyl]-4-(4-quinolinylamino)benzamide], gamma-oryzanol, and (−)-epigallocatechin gallate (ECGC), β-Thujaplicin, procainamide, valproic acid, tacedinaline, cambinol, trichostain A (TSA), verinostat, panobinostat, belinostat, RGFP-966 [(E)-N-(2-amino-4-fluorophenyl)-3-(1-cinnamyl-1H-pyrazol-4-yl) acrylamide], tubastatin A, PCI-34051 [N-Hydroxy-1-[(4-methoxyphenyl)methyl]-1H-indole-6-carboxamide], sucrose, maltose, copper, zinc, manganese, proline, asparagine, methionine, glutamine, silver nitrate, 2-aminoethoxyvinyl glycine (AVG), silver ions (Ag), 1-methylcyclopropene (1-MCP), norbornadiene, EPZ005687 [1-cyclopentyl-N-[(4,6-dimethyl-2-oxo-1H-pyridin-3-yl)methyl]-6-[4-(morpholin-4-ylmethyl)phenyl]indazole-4-carboxamide], UNC1999 [N-[(6-methyl-2-oxo-4-propyl-1H-pyridin-3-yl)methyl]-1-propan-2-yl-6-[6-(4-propan-2-ylpiperazin-1-yl) pyridin-3-yl]indazole-4-carboxamide], PF-06726304 [C₂₂H₂₁Cl₂N₃O₃], Lirametostat, Tazemetostat, EPZ011989 (C₃₅H₅₁N₅O₄·HCl), CPI-169 (C₂₇H₃₆N₄O₅S), JQ-EZ-05 (C₃₀H₃₈N₈O₂), Jasmonic acid, Methyl-jasmonate, naphthalic anhydride, salicylic acid, fenchlorazole, cloquintocet-mexyl, mefenpyr diethyl, N-[(2R)-2,3 dihydroxypropoxy]-3,4-difluoro-2-(2-fluoro-4-iodoanilino) benzamide, anthra (1,9-cd) pyrazol-6 (2H)-one:4 (4-Fluoro phenyl)-2-(4-methylsulfinylphenyl)-5-(4-pyridyl) 1H imidazole, N-benzyl-2-(pyrimidin-4-ylamino)-1,3 thiazole-4-carboxamide, Tiron, dimethylthiourea (DMTU), diphenyleneiodonium (DPI), or any combinations thereof.

In another aspect, the pre-treated seedling is a monocot seedling.

In another aspect, a method of pre-treating a seedling, comprising exposing the seedling to an increased spectrum of light, an increased heat treatment, or a cold-shock treatment, and isolating leaf explants from the exposed seedling for transformation, wherein the transformation frequency of the leaf explants from the pre-treated seedling is higher than the transformation frequency of leaf explants from an unexposed seedling is provided. In a further aspect, the heat treatment is 45° C. for 3 hours at 70% relative humidity. In another aspect, the heat treatment is 37° C. for 17 hours at 50% relative humidity. In another aspect, the pre-treated seedling is a monocot seedling.

In another aspect, a method of pre-treating a seedling with at least one chemical compound, the method comprising contacting the seedling with the at least one chemical compound, wherein the seedling is further contacted with a non-chemical pre-treatment comprising exposing the seedling to an increased spectrum of light, an increased heat treatment, or a cold-shock treatment, and isolating leaf explants from the pre-treated seedling for transformation, wherein the transformation frequency of the leaf explants from the pre-treated seedling is higher than the transformation frequency of leaf explants from an unexposed seedling is provided. In a further aspect, the pre-treatment of a seedling with the at least one chemical compound and the non-chemical pre-treatment occur simultaneously or sequentially.

### DETAILED DESCRIPTION

The disclosures herein will be described more fully hereinafter, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods pertain having the benefit of the teachings presented in the following descriptions. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

As used herein, “contacting”, “contact”, “contacted”, “comes in contact with” or “in contact with” means “direct contact” or “indirect contact”. For example, cells are placed in a condition where the cells can come into contact with an expression cassette, a nucleotide, a peptide, a RNP (ribonucleoprotein), or other substance disclosed herein. Such expression cassette, nucleotide, peptide, or other substance is allowed to be present in an environment where the cells survive (for example, medium or expressed in the cell or expressed in an adjacent cell) and can act on the cells. For example, medium comprising a selection agent may have direct contact with a cell or the medium comprising the selection agent may be separated from the cell by filter paper, plant tissues, or other cells thus, the selection agent is transferred through the filter paper, plant tissues, or other cells to the cell. The expression cassettes, nucleotides, peptides, and other substances disclosed herein may be contacted with a cell by T-DNA transfer, particle bombardment, electroporation, PEG transfection, or RNP (ribonucleoprotein) delivery.

As used herein, a “somatic embryo” is a multicellular structure that progresses through developmental stages that are similar to the development of a zygotic embryo, including formation of globular and transition-stage embryos, formation of an embryo axis and a scutellum, and accumulation of lipids and starch. Single somatic embryos derived from a zygotic embryo germinate to produce single non-chimeric plants, which may originally derive from a single cell.

As used herein, an “embryogenic callus” or “callus” is a friable or non-friable mixture of undifferentiated or partially undifferentiated cells which subtend proliferating primary and secondary somatic embryos capable of regenerating into mature fertile plants.

As used herein, “germination” is the growth of a regenerable structure, such as a somatic embryo, to form a plantlet which continues growing to produce a plant.

As used herein, a “transgenic plant” is a mature, fertile plant that contains a transgene.

The methods of the disclosure can be used to transform leaf explants. As used herein, “leaf explants” include but are not limited to radical leaves, cauline leaves, alternate leaves, opposite leaves, decussate leaves, opposite superposed leaves, whorled leaves, petiolate leaves, sessile leaves, subsessile leaves, stipulate leaves, exstipulate leaves, simple leaves, or compound leaves. Leaf explants include buds, including but not limited to lateral buds, leaf primordia, the leaf sheath, leaf base or the portion of the leaf immediately proximal to its attachment point to the petiole or stem. Such vegetative organs and their composite tissues can be used for transformation with nucleotide sequences encoding agronomically important traits. In this context, the leaf primordia contained within a mature maize seed are transformable explants.

As used herein, a “leaf” is a flat lateral structure that protrudes from a plant's stem, including the supporting stalk between the flattened leaf and the plant stem, but not including the axillary meristem located at the junction of the petiole and stem, including but not limited to a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, or a compound leaf. Once an axillary meristem is de-repressed and stimulated to begin growing, new leaf primordia and developing leaves result around it's expanding periphery and become transformable explants. In this manner, in vitro shoot proliferation can be useful to mass-produce transformable explants through hormonally-manipulated multiple shoot proliferation.

As used herein, the term “morphogenic gene” means a gene that when ectopically expressed stimulates formation of a somatically-derived structure that can produce a plant. More precisely, ectopic expression, or mutation, or silencing, or decreased expression of the morphogenic gene stimulates the de novo formation of a somatic embryo or an organogenic structure, such as a shoot meristem or an axillary meristem, that can produce a plant or stimulates regeneration of a plant. This stimulated de novo formation occurs either in the cell in which the morphogenic gene is expressed, or silenced, or repressed, or in a neighboring cell. A morphogenic gene can be a transcription factor that regulates expression of other genes, or a gene that influences hormone levels in a plant tissue, both of which can stimulate morphogenic changes. To enhance the growth rate of somatic embryos, genes that stimulate growth rates (i.e. cell division) such as GRF/GIF genes, cyclins, CDKs, or RepA, can be used. A morphogenic gene may be stably incorporated into the genome of a plant or it may be transiently expressed. In an aspect, expression of the morphogenic gene is controlled. The expression can be controlled transcriptionally or post-transcriptionally. The controlled expression may also be a pulsed expression of the morphogenic gene for a particular period of time. Alternatively, the morphogenic gene may be expressed in only some transformed cells and not expressed in others. The control of expression of the morphogenic gene can be achieved by a variety of methods as disclosed herein below. The morphogenic genes useful in the methods of the present disclosure may be obtained from or derived from any plant species.

As used herein, the term “morphogenic factor” means a morphogenic gene and/or the protein expressed by a morphogenic gene.

A morphogenic gene is involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem or axillary meristem, initiation and/or development of shoots, or a combination thereof, such as WUS/WOX genes (WUS, WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39 are useful in the methods of the disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), a CLAVATA gene (see, for example, U.S. Pat. No. 7,179,963), an Enhancer of Shoot Regeneration 1 (ESR1) gene (see Banno et al. (2001), The Plant Cell, Vol. 13:2609-2618), a Corngrass1 (Cg1) gene (see Chuck et al. (2007) Nature Genetics, Vol. 39(4):544-549), a Cup-Shaped Cotyledon (CUC) gene (see Hibara et al. (2006) The Plant Cell, Vol. 18:2946-2957), a REVOLUTA (REV) gene (see Otsuga et al. (2001) The Plant Journal 25(2):223-236), a More Axillary Growth 1 (MAX1) gene (see Stirnberg et al. (2002) Development 129:1131-1141), a SUPERSHOOT (SPS) gene (see Tanikanjana, et al. (2001) Genes & Development 15:1577-1588), a Lateral Suppressor (LAS) gene (see Greb et al. (2003) Genes & Development 17:1175-1187), a More Axillary Growth4 (MAX4) gene (see Sorefan et al. (2003) Genes & Development 17:1469-1474), a Stem Cell-Inducing Factor 1 (STEMIN1) gene (see Ishikawa et al. (2019) Nature Plants 5:681-690), a Growth-Regulating Factor 4 (GRF4) gene and/or a GRF-Interacting Factor 1 (GIF1) gene (see Debernardi et al. bioRxiv 2020.08.23.263905; doi:https://doi.org/10.1101/2020.08.23.263905), and a Growth-Regulating Factor 5 (GRF5) gene (see Kong et al. bioRxiv 2020.08.23.263947; doi:https://doi.org/10.1101/2020.08.23.263947).

Morphogenic polynucleotide sequences and amino acid sequences of functional WUS/WOX polypeptides are useful in the disclosed methods. As defined herein, a “functional WUS/WOX nucleotide” is any polynucleotide encoding a protein that contains a homeobox DNA binding domain, a WUS box, and an EAR repressor domain (Ikeda et al., 2009 Plant Cell 21:3493-3505). As demonstrated by Rodriguez et al., 2016 PNAS www.pnas.org/cgi/doi/10.1073/pnas.1607673113 removal of the dimerization sequence which leaves behind the homeobox DNA binding domain, a WUS box, and an EAR repressor domain results in a functional WUS/WOX polypeptide. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the functional WUS/WOX polypeptides useful in the methods of the present disclosure is a WUS, WUS1, WUS2, WUS3, WOX2A, WOX4, WOX5, WOX5A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX polypeptides useful in the methods of the present disclosure can be obtained from or derived from any plant including but not limited to monocots, dicots, Angiospermae, and Gymnospermae. Additional functional WUS/WOX sequences useful in the methods of the present disclosure are listed in Table 2.

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol-Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844).

As used herein, the term “transcription factor” means a protein that controls the rate of transcription of specific genes by binding to the DNA sequence of the promoter and either up-regulating or down-regulating expression. Examples of transcription factors that are also morphogenic genes, include members of the AP2/EREBP family (including BBM (ODP2)), plethora and aintegumenta sub-families, CAAT-box binding proteins such as LEC1 and HAP3, and members of the MYB, bHLH, NAC, MADS, bZIP and WRKY families.

Morphogenic polynucleotide sequences and amino acid sequences of Ovule Development Protein 2 (ODP2) polypeptides, and related polypeptides, e.g., Babyboom (BBM) protein family proteins are useful in the methods of the disclosure. In an aspect, a polypeptide comprising two AP2-DNA binding domains is an ODP2, BBM2, BMN2, or BMN3 polypeptide see, US Patent Application Publication Number 2017/0121722, herein incorporated by reference in its entirety. ODP2 polypeptides useful in the methods of the disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

ODP2 polypeptides useful in the methods of the disclosure share homology with several polypeptides within the AP2 family, e.g., see FIG. 1 of U.S. Pat. No. 8,420,893, which is incorporated herein by reference in its entirety, and provides an alignment of the maize and rice ODP2 polypeptides with eight other proteins having two AP2 domains. A consensus sequence of all proteins appearing in the alignment of U.S. Pat. No. 8,420,893 is also provided in FIG. 1 therein. The polypeptide comprising the two AP2-DNA binding domains useful in the methods of the disclosure can be obtained from or derived from any of the plants described herein. In an aspect, the polypeptide comprising the two AP2-DNA binding domains useful in the methods of the disclosure is an ODP2 polypeptide. In an aspect, the polypeptide comprising the two AP2-DNA binding domains useful in the methods of the disclosure is a BBM2 polypeptide. The ODP2 polypeptide and the BBM2 polypeptide useful in the methods of the disclosure can be obtained from or derived from any plant including but not limited to monocots, dicots, Angiospermae, and Gymnospermae. Additional Ovule Development Protein 2 (ODP2) sequences and Babyboom (BBM) (BBM, BBM1, BBM2, BBM3, BMN2, and BMN3) sequences useful in the methods of the present disclosure are listed in Table 2.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated regions).

As used herein, the term “UBI” or “UBI1” or “UBI PRO” or “UBI1 PRO” or “ZM-UBI PRO” or “ZM-UBI1 PRO” or “ZM-UBI1 PRO Complete” (SEQ ID NO: 339) is made up of the UBI1ZM PRO sequence (SEQ ID NO: 333) and the UBI1ZM 5UTR (SEQ ID NO: 334) and the UBI1ZM INTRON1 (SEQ ID NO: 335).

As used herein, the term “3×ENH” (SEQ ID NO: 340) is made up of the FMV ENH (SEQ ID NO: 336) and the PCSV ENH (SEQ ID NO: 337) and the MMV ENH (SEQ ID NO: 338).

As used herein, the term “transfer cassette” means a T-DNA comprising an expression cassette or expression cassettes flanked by the right border and the left border.

As used herein, “T-DNA” means a portion of a Ti plasmid that is inserted into the genome of a host plant cell.

As used herein, the term “selectable marker” means a transgene that when expressed in a transformed/transfected cell confers resistance to selective agents such as antibiotics, herbicides and other compounds toxic to an untransformed/untransfected cell.

As used herein, the term “EAR” means an Ethylene-responsive element binding factor-associated Amphiphilic Repression motif having general consensus sequences that act as transcriptional repression signals within transcription factors. Addition of an EAR-type repressor element to a DNA-binding protein such as a transcription factor, dCAS, or LEXA (as examples) confers transcriptional repression function to the fusion protein (Kagale, S., and Rozwadowski, K. 2010. Plant Signaling and Behavior 5:691-694).

As used herein, the term “activation domain” means the portion of a transcription factor protein that specifically confers transcriptional activation when it is brought in proximity of promoter elements such as the TATA box and Transcription Start Site (TTS). Addition of an activation domain such as the CBF1A activation domain to an endogenous DNA binding peptide, dCAS, or LEXA (as examples), positioning the activation domain near the TATA/TSS region of an endogenous genes can activate transcription (for example, Cheng et al., 2013, Cell Res 23:1163-1171; Hummel et al., US20200332307A1)

In an aspect, the methods of the disclosure comprise contacting a monocot leaf explant with a recombinant expression cassette or construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) polypeptide to produce a transgenic monocot plant comprising a heterologous polynucleotide.

In an aspect, a nucleotide sequence encoding a functional WUS/WOX polypeptide, or a nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be targeted for excision by a site-specific recombinase. Thus, the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be controlled by excision at a desired time post-transformation. It is understood that when a site-specific recombinase is used to control the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide, the expression construct comprises appropriate site-specific excision sites flanking the polynucleotide sequences to be excised, e.g., Cre lox sites if Cre recombinase is utilized. It is not necessary that the site-specific recombinase be co-located on the expression construct comprising the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide. However, in an aspect, the morphogenic gene expression cassette further comprises a nucleotide sequence encoding a site-specific recombinase.

The site-specific recombinase used to control expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be chosen from a variety of suitable site-specific recombinases. For example, in various aspects, the site-specific recombinase is FLP, FLPe, KD, Cre, SSV1, lambda Int, phi C31 Int, HK022, R, B2 (Nern et al., (2011) PNAS Vol. 108, No. 34 pp 14198-14203), B3 (Nern et al., (2011) PNAS Vol. 108, No. 34 pp 14198-14203), Gin, Tn1721, CinH, ParA, Tn5053, Bxb1, TP907-1, or U153. The site-specific recombinase can be a destabilized fusion polypeptide. The destabilized fusion polypeptide can be TETR(G17A)˜CRE or ESR(G17A)˜CRE.

In an aspect, the nucleotide sequence encoding a site-specific recombinase is operably linked to a constitutive promoter, an inducible promoter, a tissue-specific promoter, or a developmentally-regulated promoter. Suitable constitutive promoters, inducible promoters, tissue-specific promoters, and developmentally-regulated promoters include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2 (Kalla et al., 1994. Plant J. 6:849-860 and U.S. Pat. No. 5,525,716 incorporated herein by reference in its entirety), HSP17.7, HSP26, HSP18A, AT-HSP811, AT-HSP811L, GM-HSP173B, promoters activated by tetracycline, ethametsulfuron or chlorsulfuron, PLTP, PLTP1, PLTP2, PLTP3, SDR, LGL, LEA-14A, or LEA-D34 (United States Patent Application publications 20170121722 and 20180371480 incorporated herein by reference in their entireties).

In an aspect, the chemically inducible promoter operably linked to the site-specific recombinase is XVE (Zuo et al. (2002) The Plant Journal 30(3):349-359). The chemically-inducible promoter can be repressed by the tetracycline repressor (TETR), the ethametsulfuron repressor (ESR), or the chlorsulfuron repressor (CR), and de-repression occurs upon addition of tetracycline-related or sulfonylurea ligands. The repressor can be TETR and the tetracycline-related ligand is doxycycline or anhydrotetracycline. (Gatz, C., Frohberg, C. and Wendenburg, R. (1992) Stringent repression and homogeneous de-repression by tetracycline of a modified CaMV 35S promoter in intact transgenic tobacco plants, Plant J. 2, 397-404). Alternatively, the repressor can be ESR and the sulfonylurea ligand is ethametsulfuron, chlorsulfuron, metsulfuron-methyl, sulfometuron methyl, chlorimuron ethyl, nicosulfuron, primisulfuron, tribenuron, sulfosulfuron, trifloxysulfuron, foramsulfuron, iodosulfuron, prosulfuron, thifensulfuron, rimsulfuron, mesosulfuron, or halosulfuron (US20110287936 incorporated herein by reference in its entirety). An alternative method for inducible expression is use of the glucocorticoid system in which an encoded glucocorticoid repressor (Ouwerkerk et al. (2001) Planta 213:370-378) is fused to an encoded gene of interest (e.g., a morphogenic protein such as WUS2 or ODP2 protein).

In an aspect, when the morphogenic gene expression cassette or construct comprises site-specific recombinase excision sites, the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be operably linked to an auxin inducible promoter, a developmentally regulated promoter, a tissue-specific promoter, or a constitutive promoter. Exemplary auxin inducible promoters, developmentally regulated promoters, tissue-specific promoters, and constitutive promoters useful in this context include UBI, LLDAV, EVCV, DMMV, BSV (AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1 (U.S. Pat. No. 6,838,593 incorporated herein by reference in its entirety), DR5, XVE, GLB1, OLE, LTP2, HSP17.7, HSP26, HSP18A, AT-HSP811 (Takahashi, T, et al., (1992) Plant Physiol. 99 (2): 383-390), AT-HSP811L (Takahashi, T, et al., (1992) Plant Physiol. 99 (2): 383-390), GM-HSP173B (Schöffl, F., et al. (1984) EMBO J. 3(11): 2491-2497), promoters activated by tetracycline, ethametsulfuron or chlorsulfuron, PLTP, PLTP1, PLTP2, PLTP3, SDR, LGL, LEA-14A, LEA-D34 (United States Patent Application publications 20170121722 and 20180371480 incorporated herein by reference in their entireties), and any of the promoters disclosed herein.

When using a morphogenic gene cassette and a trait gene cassette (heterologous polynucleotide) to produce transgenic plants it is desirable to have the ability to segregate the morphogenic gene locus away from the trait gene (heterologous polynucleotide) locus in co-transformed plants to provide transgenic plants containing only the trait gene (heterologous polynucleotide). This can be accomplished using an Agrobacterium tumefaciens two T-DNA binary system, with two variations on this general theme (see Miller et al., 2002). For example, in the first, a two T-DNA vector, where expression cassettes for morphogenic genes and herbicide selection (i.e. HRA) are contained within a first T-DNA and the trait gene cassette (heterologous polynucleotide) is contained within a second T-DNA, where both T-DNA's reside on a single binary vector. When a plant cell is transformed by an Agrobacterium containing the two T-DNA plasmid a high percentage of transformed cells contain both T-DNA's that have integrated into different genomic locations (for example, onto different chromosomes). In the second method, for example, two Agrobacterium strains, each containing one of the two T-DNA's (either the morphogenic gene T-DNA or the trait gene (heterologous polynucleotide) T-DNA), are mixed together in a ratio, and the mixture is used for transformation. After transformation using this mixed Agrobacterium method, it is observed at a high frequency that recovered transgenic events contain both T-DNA's, often at separate genomic locations. For both co-transformation methods, it is observed that in a large proportion of the produced transgenic events, the two T-DNA loci segregate independently and progeny T1 plants can be readily identified in which the T-DNA loci have segregated away from each other, resulting in the recovery of progeny seed that contain the trait genes (heterologous polynucleotides) with no morphogenic genes/herbicide genes. See, Miller et al. Transgenic Res 11(4):381-96.

The methods provided herein provide seedling pre-treatments that enhance transformation frequency of leaf explants from the pre-treated seedlings. The methods provided herein rely upon the use of bacteria-mediated and/or biolistic-mediated gene transfer, in addition to electroporation, PEG transfection, or RNP (ribonucleoprotein) delivery to produce regenerable plant cells having an incorporated nucleotide sequence of interest. Bacterial strains useful in the methods of the disclosure include, but are not limited to, a disarmed Agrobacterium, an Ochrobactrum bacteria or a Rhizobiaceae bacteria. Disarmed Agrobacteria useful in the present methods include, but are not limited to, AGL-1, EHA105, GV3101, LBA4404, LBA4404 THY—(see U.S. Pat. No. 8,334,429 incorporated herein by reference in its entirety) and LBA4404 TD THY—in which both copies of the Tn904 transposon removed have been removed from LBA4404 THY—(see PCT/US20/24993 filed Mar. 26, 2020 which claims the benefit of U.S. Provisional Patent Application No. 62/825,054 filed on Mar. 28, 2019, all of which is hereby incorporated herein in its entirety by reference). Agrobacterium strain LBA4404 TD THY—is A. tumefaciens LBA4404 THY-strain deposited with the ATCC, assigned Accession Number PTA-10531 wherein a functional Tn904 transposon is not present or both copies of the Tn904 transposon have been deleted. Ochrobactrum bacterial strains useful in the present methods include, but are not limited to, those disclosed in U.S. Pat. Pub. No. US20180216123 incorporated herein by reference in its entirety. Rhizobiaceae bacterial strains useful in the present methods include, but are not limited to, those disclosed in U.S. Pat. No. 9,365,859 incorporated herein by reference in its entirety.

Also embodied is a plant, generated from a leaf explant wherein its seedling has been treated with various compounds, light exposures, and/or temperatures to enhance the transformability of the leaf explant. The plant comprises the described expression cassette stably incorporated into the genome of the plant. Also embodied is a seed of the plant, wherein the seed comprises the expression cassette. Further embodied is a plant wherein a gene or gene product of a heterologous polynucleotide or a polynucleotide of interest that confers a nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway. A plant wherein expression of a heterologous polynucleotide or a polynucleotide of interest alters the phenotype of said plant is also embodied.

The methods, sequences, and genes disclosed herein are useful for genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic. The methods of pre-treating seedlings described herein are used to enhance transformability of leaf explants. Leaf explants derived from pre-treated methods disclosed herein are used to increase efficiency of stable integration of heterologous polynucleotides.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The term “plant” refers to whole plants, plant organs (e.g., leaves, stems, roots, etc.), plant tissues, plant cells, plant parts, seeds, propagules, embryos, and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, meristematic regions, organogenic callus, callus tissue, protoplasts, embryos derived from mature ear-derived seed, leaves, leaf bases, leaves from mature plants, leaf tips, immature inflorescences, tassel, immature ear, silks, cotyledons, immature cotyledons, embryonic axes, cells from leaves, cells from stems, cells from roots, cells from shoots, roots, shoots, gametophytes, sporophytes, pollen, microspores, multicellular structures (MCS), regenerable plant structures (RPS), and embryo-like structures.

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

The present disclosure also includes plants obtained by any of the methods disclosed herein. The present disclosure also includes seeds from a plant obtained by any of the methods disclosed herein.

In a further aspect, the leaf explants used in the disclosed methods can be derived from pre-treated seedlings. Pre-treatment methods disclosed herein include the use of compounds (e.g., ancymidol, 2.4-D, and anti-oxidants), exposure to increased or decreased temperatures, artificial lighting (e.g., LED lights) and combinations thereof. Disclosed pre-treatment methods are used to enhance transformability of leaf explants. Disclosed pre-treatment methods may, for example, produce leaf segments that exhibit improved frequencies of T-DNA delivery, improved somatic embryo response (more rapid growth and higher numbers), increased production of TO plants, and increased single-copy integration frequencies. In other aspects, disclosed pre-treatment methods may, for example, produce leaf segments that exhibit improved transformation as the result of improved nuclease-mediated genome modification, such as when CAS-mediated cutting is used for mutagenesis, dropping-out (excision via two flanking cuts), or Homology-Dependent Repair (HDR) for precise integration into the genome.

In a further aspect, the leaf explant used in the disclosed methods can be derived from any plant, including higher plants of the Angiospermae class. Plants of the subclasses of the Monocotyledonae are suitable. Suitable species may come from the family Alliaceae, Alstroemeriaceae, Amaryllidaceae, Arecaceae, Bromeliaceae, Colchicaceae, Dioscoreaceae, Melanthiaceae, Musaceae, and Poaceae.

Suitable species from which the leaf explant used in the disclosed methods can be derived include members of the genus, Allium, Alstroemeria, Ananas, Andropogon, Arundo, Colchicum, Cynodon, Dioscorea, Elaeis, Erianthus, Festuca, Galanthus, Hordeum, Lolium, Miscanthus, Musa, Oryza, Panicum, Pennisetum, Phalaris, Phleum, Poa, Saccharum, Secale, Sorghum, Spartina, Triticosecale, Triticum, Uniola, Veratrum, and Zea.

In a further aspect, the leaf explant used in the disclosed methods can be derived from a plant that is important or interesting for agriculture, horticulture, biomass for the production of liquid fuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Zea mays (corn), Triticum aestivum (wheat), Oryza sativa (rice), Pennisetum glaucum (pearl millet), Panicum spp., Sorghum spp., Miscanthus spp., Saccharum spp., Erianthus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Triticosecale spp. (triticum—wheat X rye), Bamboo, Elaeis guineensis (palm), Musa paradisiaca (banana), Ananas comosus (pineapple), Allium cepa (onion), Colchicum autumnale, Veratrum californica., Dioscorea spp., Galanthus wornorii, Alstroemeria spp., Uniola paniculata (oats), bentgrass (Agrostis spp.), Hordeum vulgare (barley), Poa pratensis (bluegrass), Lolium spp. (ryegrass), and Phleum pratense (timothy). Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Triticosecale spp. (triticum—wheat×rye), and Bamboo; and starch-based energy crops like Zea mays (corn); and sucrose-based energy crops like Saccharum sp. (sugarcane); and biodiesel-producing energy crops like Elaeis guineensis (palm).

In a further aspect, the leaf explant from a pre-treated seedling resulting from the methods disclosed herein can be derived from any plant found within the monocot families listed in Table 1 along with representative genera and/or species.

TABLE 1

| Family Names | Order | Representative Genera/Species |
|---|---|---|
| Agavaceae | Asparagales | Agave: A. cantala (Maguey, fiber crop); Yucca |
| Alliaceae | Asparagales | Thirteen genera e.g. Allium: A. cepa (Onion, spice |
| | | crop); Ipheion, Leucocoryne, Tulbaghia |
| Amaryllidaceae | Asparagales | Fifty-nine genera e.g. Amaryllis: A. |
| | | belladona (Belladona lily, ornamental |
| | | crop); Crinum, Galanthus, Hippeastrum, Leucojum, |
| | | Lycoris, Narcissus |
| Araceae | Alismatales | Over one hundred genera e.g. Colocasia: C. esculenta |
| | | (Taro or Gabi), Alocasia, Xanthosoma (food crops, |
| | | root and tuber crops), Aglaonema, Anthurium, |
| | | Caladium, Dieffenbachia, Monstera, Philodendron, |
| | | Spathiphylum, Syngonium, Syngonium, Zantedeschia |
| | | (ornamental crops), Lemna, Pistia, Wolfia |
| Arecaceae or | Arecales | Areca: A. catechu (Betel |
| Palmae | | nut); Arenga, Cocos, Elaeis, Metroxylon, |
| | | Phoenix, Washingtonia, Lodoicea maldivica (biggest |
| | | seed), Rhapia spp. (largest leaves), Calamus (rattan) |
| Asparagaceae | Asparagales | Asparagus: A. officinalis (Vegetable Asparagus) |
| Asphodelaceae | Asparagales | Fifteen genera e.g. Aloe: A. vera (Aloe or |
| | | sabila); Asphodelus, Bulbine, Gasteria, Haworthia, |
| | | Kniphofia |
| Bromeliaceae | Poales | Ananas: A. comosus (Pineapple, fruit and fiber |
| | | crop); Aechmea, Neoregalia, Puya, Tillandsia |
| Cannaceae | Zingiberales | One genus i.e, Canna: Canna spp. |
| Colchicaceae | Liliales | Colchicum: C. autumnale (Autumn |
| | | Crocus); Burchardia |
| Commelinaceae | Commelinales | Commelina: C. diffusa (Climbing |
| | | dayflower); Rhoeo, Cyanotis, Tradescantia, Zebrina |
| Costaceae | Zingiberales | Costus: C. speciosus (Common spiral ginger) |
| Cyclanthacea | Pandanales | Carludovica palmata (fiber) |
| Cyperaceae | Poales | One hundred four genera e.g. Cyperus: C. |
| | | alternifolius, Carex, Eleocharis, Scirpus |
| Dioscoreaceae | Dioscoreales | Four genera e.g. Dioscorea: D. alata (Yam, tuber |
| | | crop); Stenomeris, Tacca, Trichopus |
| Eriocaulaceae | Poales | Ten genera e.g. Eriocaulon (pipewort), Leiothrix, |
| | | Paepalanthus, Syngonanthus |
| Heliconiaceae | Zingiberales | Heliconia: H. humilis (ornamental crop) |
| Iridaceae | Asparagales | Seventy genera e.g. Iris: I. reticulata (Reticulated Iris, |
| | | ornamental crop); Crocus, Dietes, Freesia, Gladiolus |
| Juncaceae | Poales | Seven genera e.g. Juncus: J. effusus (Soft |
| | | rush); Distichia |
| Liliaceae | Liliales | Sixteen generae.g. Lilium: L. longiflorum (White |
| | | Trumpet Lily, ornamental |
| | | crop); Tulipa, Calochortus, Erythronium, Fritillaria, |
| | | Medeola |
| Marantaceae | Zingiberales | Maranta: M. arundinacea (Arrow root, root |
| | | crop); Calathea, Thalia |
| Musaceae | Zingiberales | Three genera e.g. Musa: Musa spp. (Banana, fruit |
| | | crop; Abaca, fiber crop); Ensete, Musella |
| Orchidaceae | Asparagales | Seven to eight hundred genera |
| | | e.g. Cattleya, Cymbidium, Dendrobium, Phalaenopsis, |
| | | Vanda: V. sanderiana (waling-waling orchid, |
| | | ornamental crop), Vanilla |
| Pandanaceae | Pandanales | Three genera e.g. Pandanus: P. tectorius (Screw Pine, |
| | | fiber crop); Freycinetia |
| Poaceae or | Poales | Six hundred sixty-eight genera e.g. Avena, Hordeum, |
| Gramineae | | Sorghum, Oryza, Triticum, Zea (cereals); Bambusa, |
| | | Dendrocalamus (bamboos); Saccharum: |
| | | S. officinarum (sugarcane); Cymbopogon (lemon grass, |
| | | spice, essential-oil); Brachiaria, Cynodon, Panicum, |
| | | Pennisetum (forage crops); Axonopus, Paspalum, |
| | | Zoysia (turfgrasses) |
| Pontederiaceae | Commelinales | Pontederia: P. cordata (Pickerel |
| | | weed); Eichhornia, Heteranthera, Monochoria |
| Smilacaceae | Liliales | Smilax: S. bracteata (Sarsaparilla) |
| Strelitziaceae | Zingiberales | Three genera e.g. Strelitzia: S. reginae (Bird of |
| | | Paradise, ornamental |
| | | crop); Phenakospermum, Ravanela |
| Typhaceae | Poales | One genus: Typha: T. angustifolia (Cattail, aquatic |
| | | ornamental crop and food crop) |
| Xyridaceae | Poales | Five genera e.g. Xyris: X. pauciflora (grass-like |
| | | weed), Abolboda, Achlyphila, Aratitiyopea, Orectanth |
| Zingiberaceae | Zingiberales | Zingiber: Z. officinalis (Ginger, spice |
| | | crop), Alpinia, Curcuma, Elettaria, Hedychium |
| Zosteraceae | Alismatales | Zostera marina (eelgrass), Phyllospadix serrulatus |
| | | (surfgrass) |

In yet a further aspect, leaf explants from seedlings from the Poaceae family, including leaf explants from the sub-families Chloridoideae, Danthonioideae, Micrairoideae, Arundinoideae, Panicoideae, Aristidoideae. Oryzoideae, Bambusoideae, Pooideae, Puelioideae, Pharoideae, and Anomochlooideae are pre-treated by the methods disclosed herein and are useful in the methods of the present disclosure. Poaceae (also referred to historically as the Gramineae) is a large family of monocotyledonous flowering plants known as grasses. It includes the cereal grasses, bamboos and the grasses of natural grassland and species cultivated in lawns and pasture. Examples of species within the Poaceae useful in seedling pre-treatment methods of the present disclosure include, but are not limited to bamboo (Phyllostachys edulis), barley (Hordeum vulgare), bentgrass (Agrostis sp.), creeping bent (Agrostis stolonifera), bluegrass (Poa sp.), fescue (Festuca sp.), green bristlegrass (Setaria viridis), reed canarygrass (Phalaris arundinacea), guinea grass (Megathyrsus maximus), golden bamboo (Phyllostachys aurea), elephant grass (Arundo donax), desert grass (Stipagrostis plumosa), inland sea oats (Chasmanthium latifolium), silver grass (Miscanthus sinensis), foxtail millet (Setaria italica), finger millet (Eleusine coracana), little millet (Panicum sumatrance), kodo millet (Paspalum scrobiculatum), barnyard millet (Echinochloa frumentacea) and proso millet (Panicum miliaceum), orchard grass (Dactylis glomerata), switchgrass (Panicum virgatum), pearl millet (Pennisetum glaucum), purple false brome (Brachypodium distachyon), rice (Oryza sativa; both Japonica and indica varieties), rye (Secale cereale), ryegrass (Lolium perenne), sorghum (Sorghum bicolor), Saint Augustine grass (Stenotaphrum secundatum), sugarcane (Saccharum officinarum), teff (Eragrostis tef), fonio (Digitaria exilis), timothy (Phleum pratense), triticale (Triticosecale sp.), wheat (Triticum aestivum), durum wheat (Triticum durum), emmer wheat (Triticum dicoccum), einkorn wheat (Triticum monococcum), spelt wheat (Triticum spelta), goatgrass (Aegilops spp), wheatgrass (Agropyron cristatum), oats (Avena sativa), corn (Zea mays), teosinte (Zea mays spp. mexicana or spp. parviglumis), and perennial teosinte (Zea diploperennis).

In specific aspects, leaf explants derived from pre-treated seedlings disclosed in the methods herein and useful in the methods of the present disclosure include, but are not limited to leaf explants of bamboo (Phyllostachys edulis), barley (Hordeum vulgare), bentgrass (Agrostis sp.), creeping bent (Agrostis stolonifera), bluegrass (Poa sp.), fescue (Festuca sp.), green bristlegrass (Setaria viridis), reed canarygrass (Phalaris arundinacea), guinea grass (Megathyrsus maximus), golden bamboo (Phyllostachys aurea), elephant grass (Arundo donax), desert grass (Stipagrostis plumosa), inland sea oats (Chasmanthium latifolium), silver grass (Miscanthus sinensis), foxtail millet (Setaria italica), finger millet (Eleusine coracana), little millet (Panicum sumatrance), kodo millet (Paspalum scrobiculatum), barnyard millet (Echinochloa frumentacea) and proso millet (Panicum miliaceum), orchard grass (Dactylis glomerata), switchgrass (Panicum virgatum), pearl millet (Pennisetum glaucum), purple false brome (Brachypodium distachyon), rice (Oryza sativa; both Japonica and Indica varieties), rye (Secale cereale), ryegrass (Lolium perenne), sorghum (Sorghum bicolor), Saint Augustine grass (Stenotaphrum secundatum), sugarcane (Saccharum officinarum), teff (Eragrostis tef), fonio (Digitaria exilis), timothy (Phleum pratense), triticale (Triticosecale sp.), wheat (Triticum aestivum), durum wheat (Triticum durum), emmer wheat (Triticum dicoccum), einkorn wheat (Triticum monococcum), spelt wheat (Triticum spelta), goatgrass (Aegilops spp), wheatgrass (Agropyron cristatum), oats (Avena sativa), corn (Zea mays), teosinte (Zea mays spp. mexicana or spp. parviglumis), and perennial teosinte (Zea diploperennis).

Heterologous coding sequences, heterologous polynucleotides, and polynucleotides of interest may be used in the methods of the disclosure for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant.

General categories of heterologous polynucleotides or nucleotide sequences of interest for use in the methods of the present disclosure include, for example, those genes involved in information, such as zinc fingers, those involved in communication, such as kinases and those involved in housekeeping, such as heat shock proteins. More specific categories of transgenes (heterologous polynucleotides or nucleotide sequences of interest), for example, include genes encoding important traits for agronomics, insect resistance, disease resistance, herbicide resistance, environmental stress resistance (altered tolerance to cold, salt, drought, etc.) and grain characteristics. Still other categories of transgenes include genes for inducing expression of exogenous products such as enzymes, cofactors, and hormones from plants and other eukaryotes as well as prokaryotic organisms. It is recognized that any gene or polynucleotide of interest can be operably linked to a promoter and expressed in a plant using the methods disclosed herein.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

An “enhanced trait” as used herein describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, improved seed quality, enhanced nitrogen use efficiency, early plant growth and development, late plant growth and development, enhanced seed protein, and enhanced seed oil production.

Multiple genes of interest (heterologous polynucleotides or nucleotide sequences of interest) can be used in the methods of the disclosure and expressed in a plant, for example insect resistance traits herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, nutritional enhancement, and the like).

Such genes (heterologous polynucleotides or nucleotide sequences of interest) include, for example, Bacillus thuringiensis toxic protein genes, U.S. Pat. Nos. 5,366,892; 5,747,450; 5,736,514; 5,723,756; 5,593,881 and Geiser, et al., (1986) Gene 48:109, the disclosures of which are herein incorporated by reference in their entirety. Genes (heterologous polynucleotides or nucleotide sequences of interest) encoding disease resistance traits can also be used in the methods of the disclosure including, for example, detoxification genes, such as those which detoxify fumonisin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), herein incorporated by reference in their entirety.

Numerous trait genes (heterologous polynucleotides or nucleotide sequences of interest) are known in the art and can be used in the methods disclosed herein. By way of illustration, without intending to be limiting, trait genes that confer resistance to insects, diseases, or herbicides, trait genes that confer or contribute to an altered grain characteristic, such as altered fatty acids, altered phosphorus content, altered carbohydrates or carbohydrate composition, altered antioxidant content or composition, or altered essential seed amino acids content or composition, are examples of the types of trait genes which can be operably linked to a promoter for expression in plants transformed by the methods disclosed herein. Additional genes known in the art may be included in the expression cassettes useful in the methods disclosed herein. Non-limiting examples include genes that create a site for site specific DNA integration, genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress, or other genes and transcription factors that affect plant growth and agronomic traits such as yield, flowering, plant growth and/or plant structure.

The methods of the disclosure can be used to transform a plant with a heterologous nucleotide sequence that is an antisense sequence for a targeted gene. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

As used herein, the terms “promoter” or “transcriptional initiation region” mean a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate. It is recognized that having identified the nucleotide sequences for the promoter regions disclosed herein, it is within the state of the art to isolate and identify further promoters in the 5′ untranslated region upstream from the particular promoter regions identified herein. Additionally, chimeric promoters may be provided. Such chimeras include portions of the promoter sequence fused to fragments and/or variants of heterologous transcriptional regulatory regions. Thus, the promoter regions disclosed herein can comprise upstream promoters such as, those responsible for tissue and temporal expression of the coding sequence, enhancers and the like.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the present disclosure a post-transcriptional regulatory element may include elements that are active following transcription initiation, for example translational and transcriptional enhancers, translational and transcriptional repressors and mRNA stability determinants.

A “heterologous nucleotide sequence”, “heterologous polynucleotide of interest”, or “heterologous polynucleotide” as used throughout the disclosure, is a sequence that is not naturally occurring with or operably linked to a promoter sequence. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

It is recognized that to increase transcription levels, enhancers may be. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of promoter sequences can provide for a range of expression of a heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The transformation methods disclosed herein are useful in the genetic manipulation of any plant, thereby resulting in a change in phenotype of the transformed plant. Changes in phenotype can be accomplished by T-DNA transfer, particle bombardment, electroporation, PEG transfection, or RNP (ribonucleoprotein) delivery.

The term “operably linked” means that the transcription or translation of a heterologous nucleotide sequence is under the influence of a promoter sequence. In this manner, the nucleotide sequences for the promoters may be provided in expression cassettes along with heterologous nucleotide sequences of interest for expression in the plant of interest, more particularly for expression in the reproductive tissue of the plant.

In one aspect of the disclosure, expression cassettes comprise a transcriptional initiation region comprising a promoter nucleotide sequence or variants or fragments thereof, operably linked to a morphogenic gene and/or a heterologous nucleotide sequence. Such an expression cassette can be provided with a plurality of restriction sites for insertion of the nucleotide sequence to be under the transcriptional regulation of the regulatory regions. The expression cassette may additionally contain selectable marker genes as well as 3′ termination regions.

The expression cassette can include, in the 5′-3′ direction of transcription, a transcriptional initiation region (i.e., a promoter, or variant or fragment thereof), a translational initiation region, a heterologous nucleotide sequence of interest, a translational termination region and optionally, a transcriptional termination region functional in the host organism. The regulatory regions (i.e., promoters, transcriptional regulatory regions, and translational termination regions) and/or the polynucleotide of the aspects may be native/analogous to the host cell or to each other. Alternatively, the regulatory regions and/or the polynucleotide of the aspects may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide.

The termination region may be native with the transcriptional initiation region, may be native with the operably linked DNA sequence of interest, may be native with the plant host, or may be derived from another source (i.e., foreign or heterologous to the promoter, the DNA sequence being expressed, the plant host, or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette useful in the methods of the disclosure may also contain at least one additional nucleotide sequence for a gene, heterologous nucleotide sequence, heterologous polynucleotide of interest, or heterologous polynucleotide to be co-transformed into the organism. Alternatively, the additional nucleotide sequence(s) can be provided on another expression cassette.

Where appropriate, the nucleotide sequences may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize AdhI intron (Kyozuka, et al., (1991) Mol. Gen. Genet. 228:40-48; Kyozuka, et al., (1990) Maydica 35:353-357) and the like, herein incorporated by reference in their entirety.

The DNA expression cassettes or constructs useful in the methods of the disclosure can also include further enhancers, either translation or transcription enhancers, as may be required. These enhancer regions are well known to persons skilled in the art and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene and can be specifically modified to increase translation of the mRNA. It is recognized that to increase transcription levels enhancers may be utilized in combination with the promoter regions of the aspects. Enhancers are known in the art and include the SV40 enhancer region, the 35S enhancer element, and the like.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the expression cassettes useful in the methods of the present disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes that could serve utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

As used herein, “vector” refers to a DNA molecule such as a plasmid, cosmid or bacterial phage for introducing a nucleotide construct, for example, an expression cassette or construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the disclosure involve introducing a polypeptide or polynucleotide into a plant. As used herein, “introducing” means presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P: 175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference in their entirety. Methods and compositions for rapid plant transformation of immature embryos are also found in US 2017/0121722, herein incorporated in its entirety by reference. Vectors useful in plant transformation are found in US 2019/0078106, herein incorporated by reference in its entirety.

In specific aspects, the DNA expression cassettes or constructs can be provided to a leaf explant from a pre-treated seedling derived from the methods disclosed herein using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylenimine (PEI; Sigma #P3143).

In other aspects, the polynucleotide may be introduced into plants by contacting plants with a virus or viral nucleic acids. Generally, such methods involve incorporating a nucleotide construct within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference in their entirety.

Transformed cells derived from leaf explants of pre-treated seedlings described herein may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct, for example, an expression cassette, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the aspects containing a desired polynucleotide is cultivated using methods well known to one skilled in the art.

Methods are known in the art for the targeted insertion of a polynucleotide at a specific location in the plant genome. The insertion of the polynucleotide at a desired genomic location is achieved using a site-specific recombination system. See, for example, U.S. Pat. No. 9,222,098 B2, U.S. Pat. No. 7,223,601 B2, U.S. Pat. No. 7,179,599 B2, and U.S. Pat. No. 6,911,575 B1, all of which are herein incorporated by reference in their entirety. Briefly, a polynucleotide of interest, flanked by two non-identical recombination sites, can be contained in a T-DNA transfer cassette. The T-DNA transfer cassette is introduced into a plant having stably incorporated into its genome a target site which is flanked by two non-identical recombination sites that correspond to the sites of the transfer cassette. Alternatives to T-DNA transfer include but are not limited to, particle bombardment, electroporation, PEG transfection, or RNP (ribonucleoprotein) delivery. An appropriate recombinase is provided, and the transfer cassette is integrated at the target site. The polynucleotide of interest is thereby integrated at a specific chromosomal position in the plant genome.

In an aspect, the disclosed pre-treatment methods can be used to enhance introduction into leaf explants, polynucleotides useful to target a specific site for modification in the genome of a plant. Site specific modifications that can be introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene includes a gene that is generally coupled, associated or close to or in the vicinity of flanking CRISPR loci. The terms “Cas gene” and “CRISPR-associated (Cas) gene” are used interchangeably herein.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG which can in principle be targeted. Additional Cas endonucleases and methodologies are continually being developed in the art. It is expected that those additional Cas endonucleases and methodologies would be useful in the methods of the present disclosure.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

A “Dead-CAS9” (dCAS9) as used herein, is used to supply a transcriptional repressor domain. The dCAS9 has been mutated so that can no longer cut DNA. The dCAS9 can still bind when guided to a sequence by the gRNA and can also be fused to repressor elements. The dCAS9 fused to the repressor element, as described herein, is abbreviated to dCAS9˜REP, where the repressor element (REP) can be any of the known repressor motifs that have been characterized in plants. An expressed guide RNA (gRNA) binds to the dCAS9˜REP protein and targets the binding of the dCAS9-REP fusion protein to a specific predetermined nucleotide sequence within a promoter (a promoter within the T-DNA). For example, if this is expressed beyond—the border using a ZM-UBI PRO::dCAS9˜REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9˜REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the methods of the disclosure the variable target domain is 12, 13, 14, 15, 16, 17, 18, 19, 20, 21, 22, 23, 24, 25, 26, 27, 28, 29 or 30 nucleotides in length.

In an aspect of the methods of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including chloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that is specifically recognized and/or bound by a double-strand break inducing agent such as a LIG3-4 endonuclease (US patent publication 2009/0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology.

In an aspect, the disclosed methods can be used to introduce into plants polynucleotides useful for the targeted integration of nucleotide sequences into a plant. For example, the disclosed methods can be used to introduce T-DNA expression cassettes comprising nucleotide sequences of interest flanked by non-identical recombination sites are used to transform a plant comprising a target site. In an aspect, the target site contains at least a set of non-identical recombination sites corresponding to those on the T-DNA expression cassette. The exchange of the nucleotide sequences flanked by the recombination sites is affected by a recombinase. Thus, the disclosed methods can be used for the introduction of T-DNA expression cassettes for targeted integration of nucleotide sequences, wherein the T-DNA expression cassettes which are flanked by non-identical recombination sites recognized by a recombinase that recognizes and implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the T-DNA expression cassette.

It is recognized that the transformed plant prepared in this manner may comprise multiple target sites; i.e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known. The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and re-ligate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21:969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A. 80:4223-4227. The FLP recombinase for use in the present disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; and Shaikh et al. (1977) J. Biol. Chem. 272:5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U.S. Pat. Nos. 5,380,831,5,436,391, and Murray et al. (1989) Nucleic Acids Res. 17:477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88:3324-3328; and Murray et al. (1989) Nucleic Acids Research, 17:477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses novel FLP recombination target sites (FRT). The FRT has been identified as a minimal sequence comprising two 13 base pair repeats, separated by an eight (8) base spacer. The nucleotides in the spacer region can be replaced with a combination of nucleotides, so long as the two 13-base repeats are separated by eight nucleotides. It appears that the actual nucleotide sequence of the spacer is not critical; however, for the practice of the present disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33:12746-12751, herein incorporated by reference.

Novel FRT mutant sites can be used in the practice of the disclosed methods. Such mutant sites may be constructed by PCR-based mutagenesis. Although mutant FRT sites are known (see SEQ ID Nos 2, 3, 4 and 5 of WO1999/025821), it is recognized that other mutant FRT sites may be used in the practice of the present disclosure. The present disclosure is not restricted to the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing T-DNA expression cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

The disclosed methods also provide for means to combine multiple expression cassettes at one location within the genome. Recombination sites may be added or deleted at target sites within the genome.

Any means known in the art for bringing the three components of the system together may be used in the present disclosure. For example, a plant can be stably transformed to harbor the target site in its genome. The recombinase may be transiently expressed or provided. Alternatively, a nucleotide sequence capable of expressing the recombinase may be stably integrated into the genome of the plant. In the presence of the corresponding target site and the recombinase, the T-DNA expression cassette, flanked by corresponding non-identical recombination sites, is inserted into the transformed plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a T-DNA expression cassette containing flanking non-identical recombination sites, which correspond to those in plant one. Either plant one or plant two contains within its genome a nucleotide sequence expressing recombinase. The recombinase may be under the control of a constitutive or inducible promoter. In this manner, expression of recombinase and subsequent activity at the recombination sites can be controlled.

The disclosed methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the present disclosure. The nucleotide sequence also may encode an ‘antisense’ sequence to turn off or modify gene expression.

It is recognized that the nucleotide sequences will be utilized in a functional expression unit or T-DNA expression cassette. By functional expression unit or T-DNA expression cassette is intended, the nucleotide sequence of interest with a functional promoter, and in most instances a termination region. There are various ways to achieve the functional expression unit within the practice of the present disclosure. In one aspect of the present disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. The T-DNA expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Sequences

Sequences useful in the methods of the disclosure are presented in Table 2.

TABLE 2

| SEQ | Polynucleotide (DNA) | | |
|---|---|---|---|
| ID NO: | or Polypeptide (PRT) | NAME | DESCRIPTION |
| 1 | DNA | PHP35648 | RB + UBI1ZM PRO::UBI1ZM 5UTR::UBI1ZM |
| | | | INTRON1::LOXP::AM-CYAN1::PINII TERM + |
| | | | RAB17 PRO::MO-CRE-EXON1::ST-LS1 |
| | | | INTRON1::MO-CRE EXON2::PINII TERM + |
| | | | GZ-W64A TERM + NOS PRO::ZM- |
| | | | WUS2::PINII TERM + CAMV35S |
| | | | ENH::UBI1ZM PRO PRO::UBI1ZM |
| | | | 5UTR::UBI1ZM INTRON1::ZM-ODP2::PINII |
| | | | TERM + LOXP::ZS-YELLOW1 N1::PINII |
| | | | TERM + UBI1ZM PRO::UBI1ZM |
| | | | 5UTR::UBI1ZM INTRON1::MO-PAT::PINII |
| | | | TERM + LB |
| 2 | DNA | PHP46332 | RB + UBI1ZM 5UTR::UBI1ZM |
| | | | INTRON1::LOXP::AM-CYAN1::PINII TERM + |
| | | | RAB17 PRO::MO-CRE-EXON1::ST-LS1 |
| | | | INTRON1::MO-CRE EXON2::PINII TERM + |
| | | | NOS PRO::ZM-WUS2::PINII TERM + UBI1ZM |
| | | | PRO PRO::UBI1ZM 5UTR::UBI1ZM |
| | | | INTRON1::ZM-ODP2::PINII TERM + |
| | | | LOXP::ZS-YELLOW1 N1::PINII TERM + |
| | | | UBI1ZM PRO::UBI1ZM 5UTR::UBI1ZM |
| | | | INTRON1::MO-PAT::PINII TERM + LB |
| 3 | DNA | PHP5096 | UBI1ZM 5UTR::UBI1ZM |
| | | | INTRON1::LOXP::FLPM::PINII TERM |
| 4 | DNA | PHP71539 | VIRB1 + VIRB2 + VIRB3 + VIRB4 + VIRB5 + |
| | | | VIRB6 + VIRB7 + VIRB8 + VIRB9 + VIRB 10 + |
| | | | VIRB11 + VIRG + VIRC2 + VIRC1 + VIRD1 + |
| | | | VIRD2 + VIRD3 + VIRD4 + VIRD5 + VIRE1 + |
| | | | VIRE2 + VIRE3 + GENT + COLE1 ORI + PVS1 |
| | | | ORI |
| 5 | DNA | PHP81855 | RB + LOXP + 8xDR5 ENH::CAMV35S |
| | | | PRO::TOP3::ZM-WUS2::PINII TERM + PLTP |
| | | | PRO::TOP::ZM-ODP2::OS-T28 TERM + RAB17 |
| | | | PRO::MO-CRE-EXON1::ST-LS1 |
| | | | INTRON1::MO-CRE EXON2::PINII + LOXP + |
| | | | UBI1ZM 5UTR::UBI1ZM |
| | | | INTRON1::FRT1::NPTII::PINII TERM + |
| | | | UBI1ZM PRO::UBI1ZM 5UTR::UBI1ZM |
| | | | INTRON1::MO-PAT::PINII TERM + FRT6 + LB |
| 6 | DNA | PHP81856 | RB + LOXP + ZM-AXIG1 PRO::TOP::ZM- |
| | | | WUS2::IN2-1 TERM + PLTP PRO::TOP::ZM- |
| | | | ODP2::OS-T28 TERM + RAB17 PRO:: MO- |
| | | | CRE-EXON1::ST-LS1 INTRON1::MO-CRE |
| | | | EXON2::PINII + LOXP + UBI1ZM |
| | | | 5UTR::UBI1ZM INTRON1::FRT1::NPTII::PINII |
| | | | TERM + UBI1ZM PRO::UBI1ZM |
| | | | 5UTR::UBI1ZM INTRON1::MO-PAT::PINII |
| | | | TERM + FRT6 + LB |
| 7 | DNA | PHP81857 | RB + LOXP + NOS PRO::2xTOP::ZM- |
| | | | WUS2::IN2-1 TERM + PLTP PRO::TOP::ZM- |
| | | | ODP2::OS-T28 TERM + RAB17 PRO:: MO- |
| | | | CRE-EXON1::ST-LS1 INTRON1::MO-CRE |
| | | | EXON2::PINII + LOXP + UBI1ZM |
| | | | 5UTR::UBI1ZM INTRON1::FRT1::NPTII::PINII |
| | | | TERM + UBI1ZM PRO::UBI1ZM |
| | | | 5UTR::UBI1ZM INTRON1::AM-CYAN1::PINII |
| | | | TERM + FRT6 + LB |
| 8 | DNA | PHP81858 | RB + LOXP + NOS PRO::ZM-WUS2::IN2-1 |
| | | | TERM + PLTP PRO::TOP::ZM-ODP2::OS-T28 |
| | | | TERM + RAB17 PRO:: MO-CRE-EXON1::ST- |
| | | | LS1 INTRON1::MO-CRE EXON2::PINII + |
| | | | LOXP + UBI1ZM PRO::UBI1ZM |
| | | | 5UTR::UBI1ZM INTRON1::FRT1::NPTII::PINII |
| | | | TERM + UBI1ZM PRO::UBI1ZM |
| | | | 5UTR::UBI1ZM INTRON1::AM-CYAN1::PINII |
| | | | TERM + FRT6 + LB |
| 9 | DNA | PHP83475 | RB + LOXP + PLTP PRO::ZM-WUS2::IN2-1 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII + UBI1ZM |
| | | | PRO::UBI1ZM 5UTR::UBI1ZM INTRON1::DS- |
| | | | RED2::PINII TERM + LOXP + SB-ALS |
| | | | PRO::ZM-ALS (HRA)::PINII TERM + LB |
| 10 | DNA | PHP83621 | RB + LOXP + PLTP PRO::ZM-WUS2::IN2-1 |
| | | | TERM + AT-5-IV-2 INS + PLTP |
| | | | PRO::TOP::ZM-ODP2::OS-T28 TERM + GLB1 |
| | | | PRO::MO-CRE-EXON1::ST-LS1 |
| | | | INTRON1::MO-CRE EXON2::PINII + LOXP + |
| | | | SB-ALS PRO::ZM-ALS (HRA)::PINII TERM + |
| | | | LB |
| 11 | DNA | PHP83652 | RB + LOXP + ZM-AXIG1 PRO::ZM- |
| | | | WUS2::IN2-1 TERM + AT-5-IV-2 INS + PLTP |
| | | | PRO::TOP::ZM-ODP2::OS-T28 TERM::PINII |
| | | | TERM::CZ19B1 TERM + ZM-HSP18A |
| | | | PRO::MO-CRE-EXON1::ST-LS1 |
| | | | INTRON1::MO-CRE EXON2::PINII + SB-UBI |
| | | | PRO:: SB-UBI INTRON1::ZS-GREEN1::OS-UBI |
| | | | TERM + LOXP + SB-ALS PRO::ZM-ALS |
| | | | (HRA)::PINII TERM + LB |
| 12 | DNA | PHP89030 | ZM-PLTP PRO::ZM-ODP2::OS-T28 TERM + |
| | | | FMV ENH + PCSV ENH |
| 13 | DNA | PHP89179 | ZM-PLTP PRO::ZM-WUS2::IN2-1 TERM + |
| | | | PSW1 + GZ-W64A TERM + FL2 TERM |
| 14 | DNA | PHP90842 | RB + UBI1ZMPRO::UBI1ZM 5UTR::UBI1ZM |
| | | | INTRON1::FLPM-EXON1::ST-LS1 |
| | | | INTRON2::FLPM-EXON2::PINII TERM::AT-T9 |
| | | | TERM + FRT1::PMI::PINII TERM::CZ19B1 |
| | | | TERM + OS-ACTIN PRO::OS-ACTIN |
| | | | INTRON1::ZM-WUS2::ZM-IN2-1 TERM + |
| | | | UBI1ZMPRO::UBI1ZM 5UTR::UBI1ZM |
| | | | INTRON1::ZM-ODP2::OS-T28 TERM::GZ- |
| | | | W64A TERM:: FL2 TERM + ZM-HSP17.7 |
| | | | PRO::MO-CRE-EXON1::ST-LS1 |
| | | | INTRON1::MO-CRE EXON2::SB-CPI8 TERM + |
| | | | LOXP + SB-UBI PRO::SB-UBI INTRON1::DS- |
| | | | RED2::SB-ACTIN TERM + FRT6 + LB |
| 15 | DNA | PHP92365 | RB + LOXP + PLTP PRO::ZM-WUS2::IN2-1 |
| | | | TERM + UBI1ZMPRO::UBI1ZM |
| | | | 5UTR::UBI1ZM INTRON1::ZM-LECI::OS-UBI |
| | | | TERM + LOXP + SI-UBI3 PRO::SI-UBI3 |
| | | | INTRON1::ZS-GREEN1::PINII TERM + SB- |
| | | | ALS PRO::ZM-ALS (HRA)::SB-PEPC1 TERM + |
| | | | LB |
| 16 | DNA | PHP92765 | RB + LOXP + PLTP PRO::ZM-WUS2::IN2-1 |
| | | | TERM + ZM-PLTP2 PRO::ZM-MPKL-A::ZM |
| | | | MIRNA PRECURSOR 396H::ZM-PKL-A STAR |
| | | | SEQ::SB-GKAF TERM + ZM-HSP17.7 |
| | | | PRO::MO-CRE-EXON1::ST-LS1 |
| | | | INTRON1::MO-CRE EXON2::PINII + LOXP + |
| | | | LB |
| 17 | DNA | PHP92928 | RB + FMV ENH::PCSV ENH::MMV |
| | | | ENH::PLTP PRO::ZM-WUS2::IN2-1 TERM + |
| | | | UBI1ZMPRO::UBI1ZM 5UTR::UBI1ZM |
| | | | INTRON1::ZM-LEC1::OS-T28 TERM + SI-UBI3 |
| | | | PRO::SI-UBI3 INTRON1::ZS-GREEN1::PINII |
| | | | TERM + SB-ALS PRO::ZM-ALS (HRA)::SB- |
| | | | PEPC1 TERM + LB |
| 18 | DNA | PHP93271 | RB + PLTP PRO::ZM-WUS2::IN2-1 TERM + |
| | | | UBI1ZMPRO::UBI1ZM 5UTR::UBI1ZM |
| | | | INTRON1::REP A (WDV)::OS-T28 TERM + SB- |
| | | | UBI PRO::SB-UBI INTRON1::ZS-GREEN1::OS- |
| | | | UBI TERM + SB-ALS PRO::ZM-ALS |
| | | | (HRA)::SB-PEPC1 TERM + LB |
| 19 | DNA | PHP93559 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::IN2-1 TERM + UBI1ZM PRO::UBI1ZM |
| | | | 5UTR::UBI1ZM INTRON1::ZM-ODP2::ZM-UBI |
| | | | TERM + LOXP + SB-UBI PRO::SB-UBI |
| | | | INTRON1::ZS-GREEN1::OS-UBI TERM + SB- |
| | | | UBI PRO::SB-UBI INTRON1::PMI::SB-UBI |
| | | | TERM + LB |
| 20 | DNA | PHP93613 | RB + LOXP + ZM-HSP17.7 PRO::MO-CRE- |
| | | | EXON1::ST-LS1 INTRON1::MO-CRE |
| | | | EXON2::PINII TERM + OS-ACTIN PRO::OS- |
| | | | ACTIN INTRON1::ZM-WUS2::IN2-1 TERM + |
| | | | UBI1ZM PRO::UBI1ZM 5UTR::UBI1ZM |
| | | | INTRON1::ZM-LEC1::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 21 | DNA | PHP93696 | RB + LOXP + ZM-HSP17.7 PRO::MO-CRE- |
| | | | EXON1::ST-LS1 INTRON1::MO-CRE |
| | | | EXON2::PINII TERM + 2xOC-EME1::UBI1ZM |
| | | | PRO::UBI1ZM 5UTR::UBI1ZM |
| | | | INTRON1::LOXP::2xOC-EME1::ZM-CAB |
| | | | PRO::ZM-WUS2::IN2-1 TERM + LOXP::ZM- |
| | | | LEC1::OS-T28 TERM + SB-UBI PRO::SB-UBI |
| | | | INTRON1::ZS-GREEN1::OS-UBI TERM + SI- |
| | | | ALS PRO::SI-ALS 5UTR::ZM-ALS (HRA)::SB- |
| | | | UBI TERM + LB |
| 22 | DNA | PHP93738 | RB + LOXP + ZM-HSP17.7 PRO::MO-CRE- |
| | | | EXON1::ST-LS1 INTRON1::MO-CRE |
| | | | EXON2::PINII TERM + OS-ACTIN PRO::OS- |
| | | | ACTIN INTRON1::ZM-WUS2::ZM-IN2-1 |
| | | | TERM + UBI1ZMPRO::UBI1ZM |
| | | | 5UTR::UBI1ZM INTRON1::ZM-ODP2::OS-T28 |
| | | | TERM + LOXP + SB-UBI PRO::SB-UBI |
| | | | INTRON1::ZS-GREEN1::OS-UBI TERM + SI- |
| | | | ALS PRO::SI-ALS 5UTR::ZM-ALS (HRA)::SB- |
| | | | UBI TERM + LB |
| 23 | DNA | PHP93739 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::ZM-IN2-1 TERM + |
| | | | UBI1ZMPRO::UBI1ZM 5UTR::UBI1ZM |
| | | | INTRON1::ZM-ODP2::ZM-UBI TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 24 | DNA | PHP93743 | RB + LOXP + ZM-HSP17.7 PRO::MO-CRE- |
| | | | EXON1::ST-LS1 INTRON1::MO-CRE |
| | | | EXON2::PINII TERM + SCBV PRO::SCBV |
| | | | 5UTR::ZM-WUS2::ZM-IN2-1 TERM + |
| | | | UBI1ZMPRO::UBI1ZM 5UTR::UBI1ZM |
| | | | INTRON1::REP A (WDV)::OS-T28 TERM + |
| | | | LOXP + SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 25 | DNA | PHP93766 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::ZM-IN2-1 TERM + |
| | | | UBI1ZMPRO::UBI1ZM 5UTR::UBI1ZM |
| | | | INTRON1::ZM-LEC1::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 26 | DNA | PHP93925 | RB + UBI1ZMPRO::UBI1ZM 5UTR::UBI1ZM |
| | | | INTRON1::ZM-WUS2::ZM-IN2-1 TERM + |
| | | | FMV ENH:PCSV ENH:MMV ENH::SB-UBI |
| | | | PRO::SB-UBII INTRON1::ZM-ODP2::ZM-UBI |
| | | | TERM + SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 27 | DNA | PHP93926 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::ZM-IN2-1 TERM + |
| | | | UBI1ZMPRO::UBI1ZM 5UTR::UBI1ZM |
| | | | INTRON1::ZM-ODP2::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 28 | DNA | PHP93932 | RB + LOXP + FMV ENH::PCSV ENH::MMV |
| | | | ENH::ZM-EXP11232.1 PRO::ZM-WUS2::IN2-1 |
| | | | TERM + ZM-EXP13262.2 PRO::ZM-MPKL- |
| | | | A::ZM MIRNA PRECURSOR 396H::ZM-PKL-A |
| | | | STAR SEQ::ZM-EXP23070.1 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 29 | DNA | PHP93933 | RB + NOS PRO::ZM-WUS2::IN2-1 TERM + |
| | | | FMV ENH::PCSV ENH::MMV ENH::UBI1ZM |
| | | | PRO::UBI1ZM 5UTR::UBI1ZM INTRON1::ZM- |
| | | | ODP2::OS-T28 TERM + SB-UBI PRO::SB-UBI |
| | | | INTRON1::ZS-GREEN1::OS-UBI TERM + SB- |
| | | | ALS PRO::ZM-ALS (HRA)::SB-PEPC1 TERM + |
| | | | LB |
| 30 | DNA | PHP94331 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::ZM-IN2-1 TERM + |
| | | | UBI1ZMPRO::UBI1ZM 5UTR::UBI1ZM |
| | | | INTRON1::ZM-ODP2::ZM-UBI TERM + ZM- |
| | | | CAB PRO::ZM-CAB 5UTR::ZM-MPKL-A::ZM |
| | | | MIRNA PRECURSOR 396H::ZM-PKL-A STAR |
| | | | SEQ::ZM-EXP23070.1 TERM + LOXP + SB-UBI |
| | | | PRO::SB-UBI INTRON1::ZS-GREEN1::OS-UBI |
| | | | TERM + SI-ALS PRO::SI-ALS 5UTR::ZM-ALS |
| | | | (HRA)::SB-UBI TERM + LB |
| 31 | DNA | PHP94332 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::ZM-IN2-1 TERM + |
| | | | UBI1ZMPRO::UBI1ZM 5UTR::UBI1ZM |
| | | | INTRON1::ZM-ODP2::ZM-UBI TERM + |
| | | | UBI1ZMPRO::UBI1ZM 5UTR::UBI1ZM |
| | | | INTRON1::ZM-MPKL-A::ZM MIRNA |
| | | | PRECURSOR 396H::ZM-PKL-A STAR |
| | | | SEQ::ZM-EXP23070.1 TERM + LOXP + SB-UBI |
| | | | PRO::SB-UBI INTRON1::ZS-GREEN1::OS-UBI |
| | | | TERM + SI-ALS PRO::SI-ALS 5UTR::ZM-ALS |
| | | | (HRA)::SB-UBI TERM + LB |
| 32 | DNA | PHP94636 | RB + LOXP + 8xDR5 ENH::CAMV35S |
| | | | PRO::TOP3::ZM-WUS2::PINII TERM + ZM- |
| | | | PLTP PRO::TOP::ZM-ODP2::OS-T28 TERM + |
| | | | RAB17 PRO::MO-CRE-EXON1::ST-LS1 |
| | | | INTRON1::MO-CRE EXON2::PINII + LOXP + |
| | | | UBI1ZM 5UTR::UBI1ZM |
| | | | INTRON1::FRT1::NPTII::PINII TERM + |
| | | | UBI1ZM PRO::UBI1ZM 5UTR::UBI1ZM |
| | | | INTRON1::AM-CYAN1::PINII TERM + FRT6 + |
| | | | LB |
| 33 | DNA | PHP94682 | RB + LOXP + ZM-HSP17.7 PRO::MO-CRE- |
| | | | EXON1::ST-LS1 INTRON1::MO-CRE |
| | | | EXON2::PINII TERM + SCBV PRO::SCBV |
| | | | 5UTR::ZM-WUS2::ZM-IN2-1 TERM + |
| | | | UBI1ZMPRO::UBI1ZM 5UTR::UBI1ZM |
| | | | INTRON1::ZM-LEC1::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 34 | DNA | PHP94684 | RB + LOXP + ZM-HSP17.7 PRO::MO-CRE- |
| | | | EXON1::ST-LS1 INTRON1::MO-CRE |
| | | | EXON2::PINII TERM + IN2-1 TERM::ZM- |
| | | | WUS2::ZM-UBI1 INTRON1 (B104)::ZM-UBI1 |
| | | | 5UTR (B104)::ZM-UBI1 MINPRO (B104) + |
| | | | UBI1ZMPRO::UBI1ZM 5UTR::UBI1ZM |
| | | | INTRON1::ZM-ODP2::ZM-UBI TERM + LOXP + |
| | | | SB-UBI PRO::SB-UBI INTRON1::ZS- |
| | | | GREEN1::OS-T28 TERM + SI-ALS PRO::SI- |
| | | | ALS 5UTR::ZM-ALS (HRA)::SB-UBI TERM + |
| | | | LB |
| 35 | DNA | PHP94685 | RB + LOXP + IN2-1 TERM::ZM-WUS2::ZM- |
| | | | UBI1 INTRON1 (B104)::ZM-UBI1 5UTR |
| | | | (B104)::ZM-UBI1 MINPRO (B104)::MMV |
| | | | ENH::PCSV ENH::FMV |
| | | | ENH::UBI1ZMPRO::UBI1ZM 5UTR::UBI1ZM |
| | | | INTRON1::ZM-ODP2::ZM-UBI TERM + LOXP + |
| | | | SB-UBI PRO::SB-UBI INTRON1::ZS- |
| | | | GREEN1::OS-T28 TERM + SI-ALS PRO::SI- |
| | | | ALS 5UTR::ZM-ALS (HRA)::SB-UBI TERM + |
| | | | LB |
| 36 | DNA | PHP94715 | RB + LOXP + 8xDR5 ENH::CAMV35S |
| | | | PRO::ZM-WUS2::PINII TERM + UBI1ZM |
| | | | PRO::UBI1ZM 5UTR::UBI1ZM INTRON1::ZM- |
| | | | ODP2::PINII TERM + RAB 17 PRO::MO-CRE- |
| | | | EXON1::ST-LS1 INTRON1::MO-CRE |
| | | | EXON2::PINII + LOXP + UBI1ZM |
| | | | PRO::UBI1ZM 5UTR::UBI1ZM |
| | | | INTRON1::FRT1::NPTII::PINII TERM + |
| | | | UBI1ZM PRO::UBI1ZM 5UTR::UBI1ZM |
| | | | INTRON1::AM-CYAN1::PINII TERM + FRT6 + |
| | | | LB |
| 37 | DNA | PHP95067 | RB + LOXP + NOS PRO::ZM-WUS2::PINII |
| | | | TERM + UBI1ZM PRO::UBI1ZM |
| | | | 5UTR::UBI1ZM INTRON1::ZM-CUC1-2::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 38 | DNA | PHP95068 | RB + LOXP + NOS PRO::ZM-WUS2::PINII |
| | | | TERM + UBI1ZM PRO::UBI1ZM |
| | | | 5UTR::UBI1ZM INTRON1::ZM-CUC2::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 39 | DNA | PHP95069 | RB + LOXP + NOS PRO::ZM-WUS2::PINII |
| | | | TERM + UBI1ZM PRO::UBI1ZM |
| | | | 5UTR::UBI1ZM INTRON1::ZM-CUC3-2::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 40 | DNA | PHP95070 | RB + LOXP + NOS PRO::ZM-WUS2::PINII |
| | | | TERM + UBI1ZM PRO::UBI1ZM |
| | | | 5UTR::UBI1ZM INTRON1::ZM-GPCNAC- |
| | | | 1::SB-GKAF TERM + ZM-HSP17.7 PRO::MO- |
| | | | CRE-EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 41 | DNA | PHP95071 | RB + LOXP + NOS PRO::ZM-WUS2::PINII |
| | | | TERM + UBI1ZM PRO::UBI1ZM |
| | | | 5UTR::UBI1ZM INTRON1::AT-RKD4::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 42 | DNA | PHP95072 | RB + LOXP + FT-MEM1::NOS PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 43 | DNA | PHP95073 | RB + LOXP + ZM-PEPCI PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 44 | DNA | PHP95074 | RB + LOXP + ZM-DIURNAL 12 PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 45 | DNA | PHP95075 | RB + LOXP + NOS PRO::ZM-WUS2::PINII |
| | | | TERM + UBI1ZM PRO::UBI1ZM |
| | | | 5UTR::UBI1ZM INTRON1::AT-LEC2::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 46 | DNA | PHP95205 | RB + LOXP + RUBISCO SSU PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 47 | DNA | PHP95385 | RB + LOXP + OS-ACTIN PRO::OS-ACTIN |
| | | | INTRON1::ZM-WUS2::ZM-IN2-1 TERM + |
| | | | UBI1ZMPRO::UBI1ZM 5UTR::UBI1ZM |
| | | | INTRON1::ZM-ODP2::OS-T28 TERM::GZ- |
| | | | W64A TERM::FL2 TERM + ZM-HSP17.7 |
| | | | PRO::MO-CRE-EXON1::ST-LS1 |
| | | | INTRON1::MO-CRE EXON2::SB-CPI8 TERM + |
| | | | LOXP + SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 48 | DNA | PHP95393 | RB + LOXP + CSVMV PRO::CSVMV |
| | | | 5UTR::ZM-WUS2::PINII TERM + UBI1ZM |
| | | | PRO::UBI1ZM 5UTR::UBI1ZM INTRON1::ZM- |
| | | | ODP2::PINII TERM + ZM-HSP17.7 PRO::MO- |
| | | | CRE-EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 49 | DNA | PHP95394 | RB + LOXP + ZMEXP13262.1 PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 50 | DNA | PHP95499 | RB + LOXP + ZM-HSP17.7 PRO::MO-CRE- |
| | | | EXON1::ST-LS1 INTRON1::MO-CRE |
| | | | EXON2::PINII TERM + OS-ACTIN PRO::OS- |
| | | | ACTIN INTRON1::ZM-WUS2::ZM-IN2-1 |
| | | | TERM + UBI1ZMPRO::UBI1ZM |
| | | | 5UTR::UBI1ZM INTRON1::ZM-ODP2::OS-T28 |
| | | | TERM + LOXP + SB-UBI PRO::SB-UBI |
| | | | INTRON1::ZS-GREEN1::OS-UBI TERM + SI- |
| | | | ALS PRO::SI-ALS 5UTR::ZM-ALS (HRA)::SB- |
| | | | UBI TERM + LB |
| 51 | DNA | PHP95502 | RB + LOXP + ZM-HSP17.7 PRO::MO-CRE- |
| | | | EXON1::ST-LS1 INTRON1::MO-CRE |
| | | | EXON2::PINII TERM + AT-5-IV-2 INS + NOS |
| | | | PRO::ZM-WUS2::ZM-IN2-1 TERM + |
| | | | UBI1ZMPRO::UBI1ZM 5UTR::UBI1ZM |
| | | | INTRON1::ZM-ODP2::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 52 | DNA | PHP95503 | RB + LOXP + NOS PRO::ZM-WUS2::PINII |
| | | | TERM + UBI1ZM PRO (3X ZM-AS2 |
| | | | EME)::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + LOXP + |
| | | | SB-UBI PRO::SB-UBI INTRON1::ZS- |
| | | | GREEN1::OS-UBI TERM + SB-ALS PRO::ZM- |
| | | | ALS (HRA)::SB-UBI TERM + LB |
| 53 | DNA | PHP95881 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::AT-LEC2 |
| | | | (MO)::SB-GKAF TERM + ZM-HSP17.7 |
| | | | PRO::MO-CRE-EXON1::ST-LS1 |
| | | | INTRON1::MO-CRE EXON2::PINII TERM::OS- |
| | | | UBI TERM + LOXP + SI-UBI3 PRO::SI-UBI3 |
| | | | INTRON1::ZS-GREEN1::PINII TERM + SB- |
| | | | ALS PRO::ZM-ALS (HRA)::SB-PEPC1 TERM + |
| | | | LB |
| 54 | DNA | PHP95882 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-LEC1::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 55 | DNA | PHP95886 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::AT-RKD4::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 56 | DNA | PHP95892 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::AT-RAP2.6L::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 57 | DNA | PHP95893 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-MIR156B::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 58 | DNA | PHP95904 | RB + LOXP + NOS PRO::ZM-WUS2::PINII |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-RLD1::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 59 | DNA | PHP95987 | RB + LOXP + SCBV PRO::SCVB 5UTR::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 60 | DNA | PHP95989 | RB + LOXP + FT-PPCA1 PRO::NOS PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 61 | DNA | PHP95990 | RB + LOXP + NOS PRO::ZM-WUS2::PINII |
| | | | TERM + UBI1ZM PRO::UBI1ZM |
| | | | 5UTR::UBI1ZM INTRON1::AT-RAP2.6L |
| | | | (MO)::SB-GKAF TERM + ZM-HSP17.7 |
| | | | PRO::MO-CRE-EXON1::ST-LS1 |
| | | | INTRON1::MO-CRE EXON2::PINII TERM::OS- |
| | | | UBI TERM + LOXP + SI-UBI3 PRO::SI-UBI3 |
| | | | INTRON1::ZS-GREEN1::PINII TERM + SB- |
| | | | ALS PRO::ZM-ALS (HRA)::SB-PEPC1 TERM + |
| | | | LB |
| 62 | DNA | PHP96030 | RB + LOXP + NOS PRO::ZM-WUS2::PINII |
| | | | TERM + UBI1ZM PRO::UBI1ZM |
| | | | 5UTR::UBI1ZM INTRON1:RLD1::SB-GKAF |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 63 | DNA | PHP96031 | RB + LOXP + ZM-GOS2 PRO::ZM-GOS2 |
| | | | 5UTR1::ZM-GOS2 INTRON1::ZM-GOS2 |
| | | | 5UTR2::ZM-WUS2::PINII TERM + UBI1ZM |
| | | | PRO::UBI1ZM 5UTR::UBI1ZM INTRON1::ZM- |
| | | | ODP2::PINII TERM + ZM-HSP17.7 PRO::MO- |
| | | | CRE-EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 64 | DNA | PHP96032 | RB + LOXP + ZM-DIURNAL 11 PRO::ZM- |
| | | | WUS2::PINII TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 65 | DNA | PHP96036 | RB + LOXP + NOS PRO::ZM-WUS2::PINII |
| | | | TERM + UBI1ZM PRO::UBI1ZM |
| | | | 5UTR::UBI1ZM INTRON1::ZM-KN1::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 66 | DNA | PHP96037 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + LOXP + |
| | | | SB-UBI PRO::SB-UBI INTRON1::ZS- |
| | | | GREEN1::OS-UBI TERM + SB-ALS PRO::ZM- |
| | | | ALS (HRA)::SB-PEPC1 TERM + LB |
| 67 | DNA | PHP96277 | RB + LOXP + OS-ACTIN PRO::OS-ACTIN |
| | | | INTRON1::ZM-WUS2::IN2-1 TERM + FMV |
| | | | ENH::PCSV ENH::MMV ENH::UBI1ZM |
| | | | PRO::UBI1ZM 5UTR::UBI1ZM INTRON1::ZM- |
| | | | ODP2::OS-T28 TERM + AT-5-IV-2 INS + ZM- |
| | | | HSP17.7 PRO::MO-CRE-EXON1::ST-LS1 |
| | | | INTRON1::MO-CRE EXON2::PINII TERM + |
| | | | AT-5-IV-2 INS + LOXP + SB-UBI PRO::SB-UBI |
| | | | INTRON1::ZS-GREEN1::OS-UBI TERM + SB- |
| | | | ALS PRO::ZM-ALS (HRA)::SB-PEPC1 TERM + |
| | | | LB |
| 68 | DNA | PHP96425 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-7 INS + ZM-HSP17.7 PRO::MO-CRE- |
| | | | EXON1::ST-LS1 INTRON1::MO-CRE |
| | | | EXON2::PINII TERM + LOXP + SB-UBI |
| | | | PRO::SB-UBI INTRON1::ZS-GREEN1::OS-UBI |
| | | | TERM + SB-ALS PRO::ZM-ALS (HRA)::SB- |
| | | | PEPC1 TERM + LB |
| 69 | DNA | PHP96664 | RB + LOXP + ZM-HSP17.7 PRO::MO-CRE- |
| | | | EXON1::ST-LS1 INTRON1::MO-CRE |
| | | | EXON2::PINII TERM + NOS PRO::ZM- |
| | | | WUS2::IN2-1 TERM + UBI1ZM PRO::UBI1ZM |
| | | | 5UTR::UBI1ZM INTRON1::ZM-ODP2::OS-T28 |
| | | | TERM + SB-UBI PRO::SB-UBI INTRON1::ZS- |
| | | | GREEN1::OS-UBI TERM + SB-ALS PRO::ZM- |
| | | | ALS (HRA)::SB-PEPC1 TERM + LB |
| 70 | DNA | PHP96695 | RB + LOXP + NOS PRO::ZM-WUS2::IN2-1 |
| | | | TERM + UBI1ZM PRO (3X ZM-AS2 |
| | | | EME)::UBI1ZM INTRON1::ZM-ODP2::OS-T28 |
| | | | TERM + ZM-HSP17.7 PRO::MO-CRE- |
| | | | EXON1::ST-LS1 INTRON1::MO-CRE |
| | | | EXON2::PINII TERM + LOXP + SB-UBI |
| | | | PRO::SB-UBI INTRON1::ZS-GREEN1::OS-UBI |
| | | | TERM + SB-ALS PRO::ZM-ALS (HRA)::SB- |
| | | | UBI TERM + LB |
| 71 | DNA | PHP96716 | RB + LOXP + NOS PRO::ZM-WUS2::IN2-1 |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-KN1::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 72 | DNA | PHP96730 | RB + LOXP + ZM-SWEET11 PRO::ZM- |
| | | | WUS2::IN2-1 TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 73 | DNA | PHP96731 | RB + LOXP + ZM-DIURNAL10 PRO::ZM- |
| | | | WUS2::IN2-1 TERM + UBI1ZM PRO::UBI1ZM |
| | | | 5UTR::UBI1ZM INTRON1::ZM-ODP2::PINII |
| | | | TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 74 | DNA | PHP96751 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + LOXP + SB-UBI |
| | | | PRO::SB-UBI INTRON1::ZS-GREEN1::OS-UBI |
| | | | TERM + SB-ALS PRO::ZM-ALS (HRA)::SB- |
| | | | UBI TERM + LB |
| 75 | DNA | PHP96919 | RB + LOXP + NOS PRO (3X ZM-AS2 |
| | | | EME)::ZM-WUS2::IN2-1 TERM + UBI1ZM |
| | | | PRO::UBI1ZM 5UTR::UBI1ZM INTRON1::ZM- |
| | | | ODP2::OS-T28 TERM + ZM-HSP17.7 |
| | | | PRO::MO-CRE-EXON1::ST-LS1 |
| | | | INTRON1::MO-CRE EXON2::PINII TERM + |
| | | | LOXP + SB-UBI PRO::SB-UBI INTRON1::ZS- |
| | | | GREEN1::OS-UBI TERM + SB-ALS PRO::ZM- |
| | | | ALS (HRA)::SB-UBI TERM + LB |
| 76 | DNA | PHP96942 | RB + UBI1ZM PRO::UBI1ZM 5UTR::UBI1ZM |
| | | | INTRON1::FLPM-EXON1::ST-LS1 |
| | | | INTRON2::FLPM-EXON2::PINII TERM::AT-T9 |
| | | | TERM + FRT1::PMI::PINII TERM::CZ19B1 |
| | | | TERM + NOS PRO::ZM-WUS2::IN2-1 TERM + |
| | | | FMV ENH::PCSV ENH::MMV ENH::UBI1ZM |
| | | | PRO::UBI1ZM 5UTR::UBI1ZM INTRON1::ZM- |
| | | | ODP2::OS-T28 TERM + ZM-HSP17.7 |
| | | | PRO::MO-CRE-EXON1::ST-LS1 |
| | | | INTRON1::MO-CRE EXON2::SB-CPI8 TERM + |
| | | | LOXP + SB-UBI PRO::SB-UBI INTRON1::ZS- |
| | | | GREEN1::OS-UBI TERM + FRT6 + LB |
| 77 | DNA | PHP97334 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + LOXP + |
| | | | SB-UBI PRO::SB-UBI INTRON1::ZS- |
| | | | GREEN1::OS-UBI TERM + SB-UBII PRO::SB- |
| | | | UBI1 INTRON1::NPTII::SB-UBI TERM + LB |
| 78 | DNA | PHP97335 | RB + LOXP + SB-UBII PRO::SB-UBI1 |
| | | | INTRON1::NPTII::SB-UBI TERM + NOS |
| | | | PRO::ZM-WUS2::IN2-1 TERM + FMV |
| | | | ENH::PCSV ENH::MMV ENH::UBI1ZM |
| | | | PRO::UBI1ZM 5UTR::UBI1ZM INTRON1::ZM- |
| | | | ODP2::OS-T28 TERM + AT-5-IV-2 INS + ZM- |
| | | | HSP17.7 PRO::MO-CRE-EXON1::ST-LS1 |
| | | | INTRON1::MO-CRE EXON2::PINII TERM + |
| | | | AT-5-IV-2 INS + LOXP + SB-UBI PRO::SB-UBI |
| | | | INTRON1::ZS-GREEN1::OS-UBI TERM + LB |
| 79 | DNA | PHP97417 | RB + LOXP + UBI1ZM PRO::UBI1ZM |
| | | | 5UTR::UBI1ZM INTRON1::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 80 | DNA | PHP97453 | RB + LOXP + UBI1ZM PRO::UBI1ZM |
| | | | 5UTR::ADH1 INTRON1::ZM-WUS2::IN2-1 |
| | | | TERM + UBI1ZM PRO::UBI1ZM |
| | | | 5UTR::UBI1ZM INTRON1::ZM-ODP2::OS-T28 |
| | | | TERM::CZ19B1 TERM + ZM-HSP17.7 |
| | | | PRO::MO-CRE-EXON1::ST-LS1 |
| | | | INTRON1::MO-CRE EXON2::PINII TERM + |
| | | | LOXP + SB-UBI PRO::SB-UBI INTRON1::ZS- |
| | | | GREEN1::OS-UBI TERM + SB-ALS PRO::ZM- |
| | | | ALS (HRA)::SB-UBI TERM + LB |
| 81 | DNA | PHP97458 | RB + LOXP + FT-MEM1::NOS PRO::ZM- |
| | | | WUS2::PINII TERM + UBI1ZM PRO (3X ZM- |
| | | | AS2 EME)::UBI1ZM INTRON1::ZM-ODP2::OS- |
| | | | T28 TERM::GZ-W64A TERM::FL2 TERM + |
| | | | ZM-HSP17.7 PRO::MO-CRE-EXON1::ST-LS1 |
| | | | INTRON1::MO-CRE EXON2::PINII TERM::OS- |
| | | | UBI TERM + LOXP + SI-UBI3 PRO::SI-UBI3 |
| | | | INTRON1::ZS-GREEN1::PINII TERM + SB- |
| | | | ALS PRO::ZM-ALS (HRA)::SB-PEPC1 TERM + |
| | | | LB |
| 82 | DNA | PHP97725 | RB + LOXP + AT-5-IV-2 INS + ZM-HSP17.7 |
| | | | PRO::MO-CRE-EXON1::ST-LS1 |
| | | | INTRON1::MO-CRE EXON2::PINII TERM + |
| | | | AT-5-IV-2 INS + NOS PRO:: ZM-WUS2::IN2-1 |
| | | | TERM + AT-5-IV-2 INS + FMV ENH::PCSV |
| | | | ENH::MMV ENH::UBI1ZM PRO::UBI1ZM |
| | | | 5UTR::UBI1ZM INTRON1::ZM-ODP2::OS-T28 |
| | | | TERM + LOXP + SB-UBI PRO::SB-UBI |
| | | | INTRON1::ZS-GREEN1::OS-UBI TERM + SI- |
| | | | ALS PRO::SI-ALS 5UTR::ZM-ALS (HRA)::SB- |
| | | | UBI TERM + LB |
| 83 | DNA | PHP97726 | RB + LOXP + AT-5-IV-2 INS + ZM-HSP17.7 |
| | | | PRO::MO-CRE-EXON1::ST-LS1 |
| | | | INTRON1::MO-CRE EXON2::PINII TERM + |
| | | | NOS PRO::ZM-WUS2::IN2-1 TERM + AT-5- |
| | | | IV-2 INS + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + LOXP + |
| | | | SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 84 | DNA | PHP97933 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + |
| | | | UBI1ZM PRO::UBI1ZM 5UTR::UBI1ZM |
| | | | INTRON1::SV40 NLS::CAS9 EXON1 |
| | | | (SP)(MO)::ST-LS1 INTRON2::CAS9 EXON2 |
| | | | (SP)(MO)::VIRD2 NLS::PINII TERM + ZM-U6 |
| | | | POLIII CHR8 PRO::ZM-WXY-CR4::GUIDE |
| | | | RNA::ZM-U6 POLIII CHR8 TERM + ZM-U6 |
| | | | POLIII CHR8 PRO::ZM-WXY-CR4::GUIDE |
| | | | RNA::ZM-U6 POLIII CHR8 TERM + LOXP + |
| | | | FL2 TERM + UBI1ZM 5UTR::UBI1ZM |
| | | | INTRON1::NPTII::PINII TERM + SB-UBI |
| | | | PRO::SB-UBI INTRON1::ZS-GREEN1::OS-UBI |
| | | | TERM + LB |
| 85 | DNA | PHP98248 | RB + LOXP + NOS PRO::ZM-WUS2::IN2-1 |
| | | | TERM + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-GRF5::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 86 | DNA | PHP98283 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + |
| | | | UBI1ZM PRO::UBI1ZM 5UTR::UBI1ZM |
| | | | INTRON1::ZM-GRF5::SB-GKAF TERM + ZM- |
| | | | HSP17.7 PRO::MO-CRE-EXON1::ST-LS1 |
| | | | INTRON1::MO-CRE EXON2::PINII TERM::OS- |
| | | | UBI TERM + LOXP + SI-UBI3 PRO::SI-UBI3 |
| | | | INTRON1::ZS-GREEN1::PINII TERM + SB- |
| | | | ALS PRO::ZM-ALS (HRA)::SB-PEPC1 TERM + |
| | | | LB |
| 87 | DNA | PHP98310 | RB + LOXP + NOS PRO::ZM-WUS2::IN2-1 |
| | | | TERM + UBI1ZM PRO::UBI1ZM |
| | | | 5UTR::UBI1ZM INTRON1::ZM-GRF5::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + LOXP + |
| | | | SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 88 | DNA | PHP98392 | RB + UBI1ZM PRO::UBI1ZM 5UTR::UBI1ZM |
| | | | INTRON1::LOXP + NOS PRO::ZM-WUS2::IN2- |
| | | | 1 TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + ZM- |
| | | | HSP17.7 PRO::MO-CRE-EXON1::ST-LS1 |
| | | | INTRON1::MO-CRE EXON2::PINII TERM::OS- |
| | | | UBI TERM + LOXP::ZM-GRF5::SB-GKAF |
| | | | TERM + SB-UBI PRO::SB-UBI INTRON1::ZS- |
| | | | GREEN1::OS-UBI TERM + SI-ALS PRO::SI- |
| | | | ALS 5UTR::ZM-ALS (HRA)::SB-UBI TERM + |
| | | | LB |
| 89 | DNA | PHP98393 | RB + FMV ENH::PCSV ENH::MMV |
| | | | ENH::UBI1ZM PRO::UBI1ZM 5UTR::UBI1ZM |
| | | | INTRON1::LOXP::ZM-ODP2::OS-T28 |
| | | | TERM:TAV-T2A:ZM-WUS2::IN2-1 TERM + |
| | | | AT-5-IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + |
| | | | LOXP:: ZM-GRF5::SB-GKAF TERM + SB-UBI |
| | | | PRO::SB-UBI INTRON1::ZS-GREEN1::OS-UBI |
| | | | TERM + SI-ALS PRO::SI-ALS 5UTR::ZM-ALS |
| | | | (HRA)::SB-UBI TERM + LB |
| 90 | DNA | PHP98407 | RB + LOXP + UBI1ZM PRO::UBI1ZM |
| | | | 5UTR::UBI1ZM INTRON1::ZM-GRF5::SB- |
| | | | GKAF TERM + ZM-HSP17.7 PRO::MO-CRE- |
| | | | EXON1::ST-LS1 INTRON1::MO-CRE |
| | | | EXON2::PINII TERM::OS-UBI TERM + |
| | | | LOXP:: +SI-UBI3 PRO::SI-UBI3 INTRON1::ZS- |
| | | | GREEN1::PINII TERM + SB-ALS PRO::ZM- |
| | | | ALS (HRA)::SB-PEPC1 TERM + LB |
| 91 | DNA | PHP98784 | RB + LOXP + NOS PRO::ZM-WUS2::IN2-1 |
| | | | TERM + FMV ENH::PCSV ENH::MMV |
| | | | ENH::UBI1ZM PRO::UBI1ZM 5UTR::UBI1ZM |
| | | | INTRON1::ZM-ODP2::OS-T28 TERM + AT-5- |
| | | | IV-2 INS + ZM-HSP17.7 PRO::MO-CRE- |
| | | | EXON1::ST-LS1 INTRON1::MO-CRE |
| | | | EXON2::PINII TERM + AT-5-IV-2 INS + LOXP + |
| | | | UBI1ZM PRO::UBI1ZM 5UTR::UBI1ZM |
| | | | INTRON1::SV40 NLS::CAS9 EXON1 |
| | | | (SP)(MO)::ST-LS1 INTRON2::CAS9 EXON2 |
| | | | (SP)(MO)::VIRD2 NLS:: PINII TERM + ZM-U6 |
| | | | POLIII CHR8 PRO::ZM-WXY-CR4::GUIDE |
| | | | RNA::ZM-U6 POLIII CHR8 TERM + ZM-U6 |
| | | | POLIII CHR8 PRO::ZM-WXY-CR4::GUIDE |
| | | | RNA::ZM-U6 POLIII CHR8 TERM + FL2 TERM + |
| | | | UBI1ZM 5UTR::UBI1ZM |
| | | | INTRON1::NPTII::PINII TERM + SB-UBI |
| | | | PRO::SB-UBI INTRON1::ZS-GREEN1::OS-UBI |
| | | | TERM + LB |
| 92 | DNA | PHP8418- | FRT1::PMI::PINII TERM::CZ19B1 TERM + |
| | | 0004 | UBI1ZM PRO::UBI1ZM 5UTR::UBI1ZM |
| | | | INTRON1::DS-RED2::PINII TERM + FRT6 |
| 93 | DNA | PHP54733 | RB + LOXP + RAB17 PRO:: MO-CRE- |
| | | | EXON1::ST-LS1 INTRON1::MO-CRE |
| | | | EXON2::PINII TERM + NOS PRO::ZM- |
| | | | WUS2::PINII TERM + UBI1ZM PRO |
| | | | PRO::UBI1ZM 5UTR::UBI1ZM INTRON1::ZM- |
| | | | ODP2::PINII TERM + LOXP + UBI1ZM PRO |
| | | | PRO::UBI1ZM 5UTR::UBI1ZM INTRON1::ZS- |
| | | | GREEN1::PINII TERM::SB-ACTIN TERM_SB- |
| | | | UBI PRO::SB-INTRON1::PMI::SB-UBI TERM + |
| | | | FRT87 + LB |
| 94 | DNA | ATPeF1D | Promoter and 5UTR sequence from Gene Model |
| | | PRO | GRMZM2G171628 |
| 95 | DNA | EIF4a PRO | Promoter and 5UTR sequence from Gene Model |
| | | | GRMZM2G116034 |
| 96 | DNA | RRM PRO | Promoter and 5UTR sequence from Gene Model |
| | | | GRMZM2G102829 |
| 97 | DNA | EF1A PRO | Promoter and 5UTR sequence from Gene Model |
| | | | GRMZM2G343543 |
| 98 | DNA | RPL10A | Promoter and 5UTR sequence from Gene Model |
| | | PRO | GRMZM2G144387 |
| 99 | DNA | APX2 PRO | Promoter and 5UTR sequence from Gene Model |
| | | | GRMZM2G140667 |
| 100 | DNA | VDAC1a | Promoter and 5UTR sequence from Gene Model |
| | | PRO | GRMZM2G150616 |
| 101 | DNA | EF1A-Tu | Promoter and 5UTR sequence from Gene Model |
| | | PRO | GRMZM2G153541 |
| 102 | DNA | LEA-14 PRO | Promoter and 5UTR sequence from Gene Model |
| | | | GRMZM2G352415 |
| 103 | DNA | RP-S7 PRO | Promoter and 5UTR sequence from Gene Model |
| | | | GRMZM2G156673 |
| 104 | DNA | RP-L5 PRO | Promoter and 5UTR sequence from Gene Model |
| | | | GRMZM5G815894 |
| 105 | DNA | ENO2 PRO | Promoter and 5UTR sequence from Gene Model |
| | | | GRMZM2G048371 |
| 106 | DNA | RP-L28 PRO | Promoter and 5UTR sequence from Gene Model |
| | | | GRMZM2G088060 |
| 107 | DNA | OS-ACTIN | Promoter sequence from rice actin |
| | | PRO | |
| 108 | DNA | ZM-UBI2 | Promoter and 5UTR sequence from |
| | | PRO | GRMZM2G419891 |
| 109 | DNA | UBI1ZM | Promoter and 5UTR sequence from UBI1ZM PRO |
| | | PRO | |
| 110 | DNA | GRP1 PRO | Promoter and 5UTR sequence from Gene Model |
| | | | GRMZM2G080603 |
| 111 | DNA | RP-L1 PRO | Promoter and 5UTR sequence from Gene Model |
| | | | GRMZM2G144387 |
| 112 | DNA | DNAJ2 PRO | Promoter and 5UTR sequence from Gene Model |
| | | | GRMZM2G364069 |
| 113 | DNA | SAMDC2 | Promoter and 5UTR sequence from Gene Model |
| | | | GRMZM2G154397 |
| 114 | DNA | CPPIase | Promoter and 5UTR sequence from Gene Model |
| | | PRO | GRMZM2G326111 |
| 115 | DNA | ZM-GRF5 | Maize Growth Regulating Factor5 coding |
| | | | sequence |
| 116 | PRT | ZM-GRF5 | Maize Growth Regulating Factor5 encoded protein |
| 117 | DNA | ZM-GRF4 | Maize Growth Regulating Factor4 coding |
| | | | sequence |
| 118 | PRT | ZM-GRF4 | Maize Growth Regulating Factor4 encoded protein |
| 119 | DNA | ZM-GIF1 | Maize GRF-Interacting Factor1 coding sequence |
| 120 | PRT | ZM-GIF1 | Maize GRF-Interacting Factor1 encoded protein |
| 121 | DNA | GRF4~GIF1 | Fusion between GRF4 and GIF1 coding sequences |
| 122 | PRT | GRF4~GIF1 | Encoded protein for fusion between GRF4 and |
| | | | GIF1 gene |
| 123 | DNA | Corngrass1 | ZM-MIR156B, MicroRNA156b also known as |
| | | (Cg1) | Corngrass1 |
| 124 | DNA | ZM- | Zea mays ortholog of Physcomitrella patens |
| | | STEMIN1 | STEMIN1 gene |
| 125 | DNA | ZM- | Zea mays ortholog of the Arabidopsis |
| | | REVOLUTA | REVOLUTA gene |
| 126 | DNA | ZM-ESR1 | Zea mays ortholog of the AT-ESRI gene |
| 127 | DNA | ZM-LAS1 | Zea mays ortholog of AT-LAS1 gene |
| 128 | DNA | ZM-CUC3 | Zea mays ortholog of the Arabidopsis AT-CUC3 |
| | | | gene |
| 129 | DNA | ZM- | Maize ortholog of the Arabidopsis |
| | | SuperShoot1 | SUPERSHOOT1 gene |
| 130 | DNA | ZM-MAX1 | Maize ortholog of the Arabidopsis MORE |
| | | | AXILLARY GROWTH1 gene |
| 131 | DNA | ZM-MAX4 | Maize ortholog of Arabidopsis MORE |
| | | | AXILLARY GROWTH4 gene |
| 132 | DNA | ZM-MIR- | Micro-RNA sequence used to target the transcript |
| | | SPS1 | of the SuperShoot1 gene |
| 133 | DNA | ZM-MIR- | Micro-RNA sequence used to target the transcript |
| | | MAX1 | of the Zm-MAX1 gene |
| 134 | DNA | ZM-MIR- | Micro-RNA sequence used to target the transcript |
| | | MAX4 | of the Zm-MAX4 gene |
| 135 | DNA | ZM-CUC1 | Zea mays ortholog of the Arabidopsis CUC1 gene |
| 136 | DNA | Plasmid C | Expression cassettes for Cas9 and gRNA for |
| | | | Targeted SDN2 |
| 137 | DNA | Plasmid B | Donor template sequence for Targeted SDN2 |
| 138 | DNA | Plasmid D | Expression cassette 3xENH::UBI PRO::ZM- |
| | | | ODP2::OS-T28 TERM for Targeted SDN2 |
| 139 | DNA | Plasmid E | Expression cassette OS-ACTIN PRO::ZM- |
| | | | WUS2::PINII TERM for Targeted SDN2 |
| 140 | DNA | GRF5~GIF1 | Fusion between GRF5 and GIF1 coding sequences |
| | | | (GRF5-GIF1) with intervening sequence encoding |
| | | | a flexible polylinker |
| 141 | PRT | GRF5~GIF1 | Encoded protein for fusion between GRF5 and |
| | | | GIF1 gene (GRF4-GIF1) with intervening |
| | | | sequence encoding a flexible polylinker |
| 142 | DNA | ZM-CUC2 | Zea mays ortholog of the Arabidopsis CUC2 gene |
| 143 | DNA | AT-WUS | Arabidopsis thaliana WUS coding sequence |
| 144 | PRT | AT-WUS | Arabidopsis thaliana WUS protein sequence |
| 145 | DNA | LJ-WUS | Lotus japonicus WUS coding sequence |
| 146 | PRT | LJ-WUS | Lotus japonicus WUS protein sequence |
| 147 | DNA | GM-WUS | Glycine max WUS coding sequence |
| 148 | PRT | GM-WUS | Glycine max WUS protein sequence |
| 149 | DNA | CS-WUS | Camelina sativa WUS coding sequence |
| 150 | PRT | CS-WUS | Camelina sativa WUS protein sequence |
| 151 | DNA | CR-WUS | Capsella rubella WUS coding sequence |
| 152 | PRT | CR-WUS | Capsella rubella WUS protein sequence |
| 153 | DNA | AA-WUS | Arabis alpina WUS coding sequence |
| 154 | PRT | AA-WUS | Arabis alpina WUS protein sequence |
| 155 | DNA | RS-WUS | Raphanus sativus WUS coding sequence |
| 156 | PRT | RS-WUS | Raphanus sativus WUS protein sequence |
| 157 | DNA | BN-WUS | Brassica napus WUS coding sequence |
| 158 | PRT | BN-WUS | Brassica napus WUS protein sequence |
| 159 | DNA | BO-WUS | Brassica oleracea var. oleracea WUS coding |
| | | | sequence |
| 160 | PRT | BO-WUS | Brassica oleracea var. oleracea WUS protein |
| | | | sequence |
| 161 | DNA | HA-WUS | Helianthus annuus WUS coding sequence |
| 162 | PRT | HA-WUS | Helianthus annuus WUS protein sequence |
| 163 | DNA | PT-WUS | Populus trichocarpa WUS coding sequence |
| | | (POPTR- | |
| | | WUS) | |
| 164 | PRT | PT-WUS | Populus trichocarpa WUS protein sequence |
| | | (POPTR- | |
| | | WUS) | |
| 165 | DNA | VV-WUS | Vitis vinifera WUS coding sequence |
| | | (VITVI- | |
| | | WUS) | |
| 166 | PRT | VV-WUS | Vitis vinifera WUS protein sequence |
| | | (VITVI- | |
| | | WUS) | |
| 167 | DNA | AT-WUS | Arabidopsis thaliana WUS coding sequence (soy |
| | | | optimized) |
| 168 | PRT | AT-WUS | Arabidopsis thaliana WUS protein sequence |
| 169 | DNA | LJ-WUS | Lotus japonicus WUS coding sequence (soy |
| | | | optimized) |
| 170 | PRT | LJ-WUS | Lotus japonicus WUS protein sequence |
| 171 | DNA | MT-WUS | Medicago truncatula WUS coding sequence (soy |
| | | | optimized) |
| 172 | PRT | MT-WUS | Medicago truncatula WUS protein sequence |
| 173 | DNA | PH-WUS | Petunia × hybrida WUS coding sequence (soy |
| | | (PETHY- | optimized) |
| | | WUS) | |
| 174 | PRT | PH-WUS | Petunia × hybrida WUS protein sequence |
| | | (PETHY- | |
| | | WUS) | |
| 175 | DNA | PV-WUS | Phaseolus vulgaris WUS coding sequence (soy |
| | | | optimized) |
| 176 | PRT | PV-WUS | Phaseolus vulgaris WUS protein sequence |
| 177 | DNA | ZM-WUS1 | Zea mays WUS1 coding sequence |
| 178 | PRT | ZM-WUS1 | Zea mays WUS1 protein sequence |
| 179 | DNA | ZM-WUS2 | Zea mays WUS2 coding sequence |
| 180 | PRT | ZM-WUS2 | Zea mays WUS2 protein sequence |
| 181 | DNA | ZM-WUS3 | Zea mays WUS3 coding sequence |
| 182 | PRT | ZM-WUS3 | Zea mays WUS3 protein sequence |
| 183 | DNA | ZM-WOX2A | Zea mays WOX2A coding sequence |
| 184 | PRT | ZM-WOX2A | Zea mays WOX2A protein sequence |
| 185 | DNA | ZM-WOX4 | Zea mays WOX4 coding sequence |
| 186 | PRT | ZM-WOX4 | Zea mays WOX4 protein sequence |
| 187 | DNA | ZM-WOX5A | Zea mays WOX5A coding sequence |
| 188 | PRT | ZM-WOX5A | Zea mays WOX5A protein sequence |
| 189 | DNA | ZM-WOX9 | Zea mays WOX9 coding sequence |
| 190 | PRT | ZM-WOX9 | Zea mays WOX9 protein sequence |
| 191 | DNA | GG-WUS | Gnetum gnemon WUS coding sequence |
| | | (GNEGN- | |
| | | WUS) | |
| 192 | PRT | GG- | Gnetum gnemon WUS protein sequence |
| | | WUS(GNEG | |
| | | N-WUS) | |
| 193 | DNA | MD-WUS | Malus domestica WUS coding sequence |
| | | (MALDO- | |
| | | WUS) | |
| 194 | PRT | MD-WUS | Malus domestica WUS protein sequence |
| | | (MALDO- | |
| | | WUS) | |
| 195 | DNA | ME-WUS | Manihot esculenta WUS coding sequence |
| | | (MANES- | |
| | | WUS) | |
| 196 | PRT | ME-WUS | Manihot esculenta WUS protein sequence |
| | | (MANES- | |
| | | WUS) | |
| 197 | DNA | KF-WUS | Kalanchoe fedtschenkoi WUS coding sequence |
| | | (KALFE- | |
| | | WUS) | |
| 198 | PRT | KF-WUS | Kalanchoe fedtschenkoi WUS protein sequence |
| | | (KALFE- | |
| | | WUS) | |
| 199 | DNA | GH-WUS | Gossypium hirsutum WUS coding sequence |
| | | (GOSHI- | |
| | | WUS) | |
| 200 | PRT | GH-WUS | Gossypium hirsutum WUS protein sequence |
| | | (GOSHI- | |
| | | WUS) | |
| 201 | DNA | ZOSMA- | Zostera marina WUS coding sequence |
| | | WUS | |
| 202 | PRT | ZOSMA- | Zostera marina WUS protein sequence |
| | | WUS | |
| 203 | DNA | AMBTR- | Amborella trichopoda WUS coding sequence |
| | | WUS | |
| 204 | PRT | AMBTR- | Amborella trichopoda WUS protein sequence |
| | | WUS | |
| 205 | DNA | AC-WUS | Aquilegia coerulea WUS coding sequence |
| | | (AQUCO- | |
| | | WUS) | |
| 206 | PRT | AC-WUS | Aquilegia coerulea WUS protein sequence |
| | | (AQUCO- | |
| | | WUS) | |
| 207 | DNA | AH-WUS | Amaranthus hypochondriacus WUS coding |
| | | (AMAHY- | sequence |
| | | WUS) | |
| 208 | PRT | AH-WUS | Amaranthus hypochondriacus WUS protein |
| | | (AMAHY- | sequence |
| | | WUS) | |
| 209 | DNA | CUCSA- | Cucumis sativus WUS coding sequence |
| | | WUS | |
| 210 | PRT | CUCSA - | Cucumis sativus WUS protein sequence |
| | | WUS | |
| 211 | DNA | PINTA- | Pinus taeda WUS coding sequence |
| | | WUS | |
| 212 | PRT | PINTA- | Pinus taeda WUS protein sequence |
| | | WUS | |
| 213 | DNA | SL-WUS | WUS ortholog of Solanum lycopersicum with |
| | | | KpnI site replaced by changing C to T at 762 bp |
| | | | coding sequence |
| 214 | PRT | SL-WUS | Solanum lycopersicum WUS protein sequence |
| 215 | DNA | ZM-ODP2 | Z. mays ODP2 coding sequence |
| 216 | PRT | ZM-ODP2 | Z. mays ODP2 protein sequence |
| 217 | DNA | ZM-BBM2 | Z. mays BBM2 coding sequence |
| 218 | PRT | ZM-BBM2 | Z. mays BBM2 protein sequence |
| 219 | DNA | ZM-ODP2 | Z. mays ODP2 coding sequence (synthetic) |
| 220 | DNA | OS-BBM1 | Oryza sativa BBM1 coding sequence |
| 221 | PRT | OS-BBM1 | Oryza sativa BBM1 protein sequence |
| 222 | DNA | OS-BBM2 | Oryza sativa BBM2 coding sequence |
| 223 | PRT | OS-BBM2 | Oryza sativa BBM2 protein sequence |
| 224 | DNA | OS-BBM3 | Oryza sativa BBM3 coding sequence |
| 225 | PRT | OS-BBM3 | Oryza sativa BBM3 protein sequence |
| 226 | DNA | SB-BBM2 | Sorghum bicolor BBM2 coding sequence |
| 227 | PRT | SB-BBM2 | Sorghum bicolor BBM2 protein sequence |
| 228 | DNA | SB-ODP2 | Sorghum bicolor ODP2 coding sequence |
| 229 | PRT | SB-ODP2 | Sorghum bicolor ODP2 protein sequence |
| 230 | DNA | SI-ODP2 | Setaria italica ODP2 coding sequence |
| 231 | PRT | SI-ODP2 | Setaria italica ODP2 protein sequence |
| 232 | DNA | 234 | Brachypodium distachyom ODP2 coding sequence |
| 233 | PRT | BD-ODP2 | Brachypodium distachyom ODP2 protein sequence |
| 234 | DNA | SB-ODP2 | Sorghum bicolor ODP2 genomic sequence |
| 235 | DNA | PHP8418- | RB + LOXP + NOS PRO::WUS2::IN2-1 TERM + |
| | | 0005 | FMV ENH::PVSC ENH::MMV ENH::UBI1ZM |
| | | | PRO::UBI1ZM 5UTR::UBI1ZM |
| | | | INTRON1::ZM-ODP2::OS-T28 TERM + AT5- |
| | | | IV-7 INS + HSP17.7 PRO::MO-CRE |
| | | | EXON1::ST-LS1 INTRON1::MO-CRE |
| | | | EXON2::PINII TERM + OS-UBI TERM + LOXP + |
| | | | UBI1ZM PRO::UBI1ZM 5UTR::UBI1ZM |
| | | | INTRON1::SV40 NLS::CAS9 EXON1::ST-LS1 |
| | | | INTRON1::CAS9 EXON2::VIRD2 NLS::ZM- |
| | | | UBI TERM + ZM-U6 POLII CHR8 PRO::ZM- |
| | | | CHR1-53.66-45CR1::GUIDE RNA::ZM-U6 |
| | | | POLII CHR8 PRO TERM + ZM-ALS PRO::ZM- |
| | | | ALS (HRA)-V2::SB-UBI TERM + ZM CHR1- |
| | | | 53.66-45CR1 TARGET SITE + ZM-SEQ11 |
| | | | (GENOMIC)(EDH5G) + SB-UBI |
| | | | PRO::NPTII::SI-UBI TERM (MOD1) + ZM- |
| | | | SEQ12 (GENOMIC)(EDH5G) + ZM CHR1- |
| | | | 53.66-45CR1 TARGET SITE + SB-UBI |
| | | | PRO::ZS-GREEN::OS-UBI TERM + LB |
| 236 | DNA | PHP8418- | RB + LOXP + NOS PRO::WUS2::IN2-1 TERM + |
| | | 0006 | FMV ENH::PVSC ENH::MMV ENH::UBI1ZM |
| | | | PRO::UBI1ZM 5UTR::UBI1ZM |
| | | | INTRON1::ZM-ODP2::OS-T28 TERM LOXP + |
| | | | AT5-IV-7 INS + HSP17.7 PRO::MO-CRE |
| | | | EXON1::ST-LS1 INTRON1::MO-CRE |
| | | | EXON2::PINII TERM + OS-UBI TERM + |
| | | | UBI1ZM PRO::UBI1ZM 5UTR::UBI1ZM |
| | | | INTRON1::SV40 NLS::CAS9 EXON1:: ST-LS1 |
| | | | INTRON1::CAS9 EXON2::VIRD2 NLS::ZM- |
| | | | UBI TERM + ZM-U6 POLII CHR8 PRO::ZM- |
| | | | CHR1-53.66-45CR1::GUIDE RNA:: ZM-U6 |
| | | | POLII CHR8 PRO TERM + ZM-ALS PRO::ZM- |
| | | | ALS (HRA)-V2::SB-UBI TERM + ZM CHR1- |
| | | | 53.66-45CR1 TARGET SITE + ZM-SEQ11 |
| | | | (GENOMIC)(EDH5G) + SB-UBI |
| | | | PRO::NPTII::SI-UBI TERM (MOD1) + ZM- |
| | | | SEQ12 (GENOMIC)(EDH5G) + ZM CHR1- |
| | | | 53.66-45CR1 TARGET SITE + SB-UBI |
| | | | PRO::ZS-GREEN::OS-UBI TERM + LB |
| 237 | DNA | PHP70298 | VIRB1 + VIRB2 + VIRB3 + VIRB4 + VIRB5 + |
| | | | VIRB6 + VIRB7 + VIRB8 + VIRB9 + VIRB10 + |
| | | | VIRB11 + VIRG + VIRC2 + VIRC1 + VIRD1 + |
| | | | VIRD2 + GENT + COLE1 ORI + PVS1 ORI |
| 238 | DNA | RV005393 | VIRA + VIRJ + VIRB1 + VIRB2 + VIRB3 + |
| | | | VIRB4 + VIRB5 + VIRB6 + VIRB7 + VIRB8 + |
| | | | VIRB9 + VIRB10 + VIRB11 + VIRG-V1 + |
| | | | VIRC2 + VIRC1(FL) + VIRD1 + VIRD2 + |
| | | | VIRD3 + VIRD4 + VIRD5 + VIRE1 + VIRE2 + |
| | | | VIRE3 + GENT + ORI V + CTL + TRF A + |
| | | | PARDE |
| 239 | DNA | RV007497 | AR-VIRA-ALT1 + AR-VIRB1 + AR-VIRB2 + |
| | | | AR-VIRB3 + AR-VIRB4-ALT1 + AR-VIRB5-V1 + |
| | | | AR-VIRB6 + AR-VIRB7 + AR-VIRB8 + AR- |
| | | | VIRB9 + AR-VIRB10 + AR-VIRB11 + AR-VIRG + |
| | | | AR-VIRC2 + AR-VIRC1-V1 + AR-VIRD1 + |
| | | | AR-VIRD2 + AR-VIRD3-V1 + AR-VIRD4 + AR- |
| | | | VIRD5-ALT1 + AR-VIRF + AR-VIRE3 + AR- |
| | | | GALLS-V1 + GENT + COLE1 ORI + PVS1 ORI |
| 240 | DNA | PHP71193 | UBI1ZM PRO::UBI1ZM 5UTR:: UBI1ZM |
| | | | INTRON1::SV40 NLS::CAS9 EXON1:: ST-LS1 |
| | | | INTRON1::CAS9 EXON2::VIRD2 NLS::PINII |
| | | | TERM + ZM-U6 POLIII CHR8 PRO::ZM-CHR1- |
| | | | 52.56-8CR1::GUIDE RNA:: ZM-U6 POLIII |
| | | | CHR8 PRO TERM + PUC ORI + AMP + F1 ORI |
| 241 | DNA | PHP71788 | RB + ZM-SEQ80 + LOXP + UBI1ZM |
| | | | PRO::UBI1ZM 5UTR::UBI1ZM |
| | | | INTRON1::FRT1 + NPTII::PINII TERM + |
| | | | FRT87 + ZM-SEQ81 + LB |
| 242 | DNA | PHP21875 | UBI1ZM PRO::UBI1ZM 5UTR:: UBI1ZM |
| | | | INTRON1::ZM-ODP2::PINII TERM + PUC ORI + |
| | | | KAN + F1 ORI |
| 243 | DNA | PHP40428 | RB + H2B PRO:: UBI1ZM 5UTR:: UBI1ZM |
| | | | INTRON1::ZS-GREEN::PINII TERM + DMMV |
| | | | PRO::DMMV5UTR:: |
| | | | ATTB4::UNISCN-22::ATTB3::ST-LS1 |
| | | | INTRON2::ATTB3::UNISCN-22::ATTB4::NOS |
| | | | TERM + LB |
| 244 | DNA | PHP93586 | RB + PLTP PRO::ZM-WUS2::TAV- |
| | | | T2A::REPA::OS-T28 TERM + SB-UBI PRO::SB- |
| | | | UBI INTRON1::ZSGREEN1::OS-UBI TERM + |
| | | | SB-ALS PRO::ZM-ALS::SB-PEPC1 TERM + LB |
| 245 | DNA | PHP93742 | RB + LOXP + ZM-EXP13262.1 PRO::ZM- |
| | | | WUS2::TAV-T2A::ZM-ODP2::IN2 TERM + ZM- |
| | | | EXP11232.1 PRO::ZM-MPKL-A::ZM MiRNA |
| | | | PRECURSOR 396H::ZM-MPKL-A STAR |
| | | | SEQ::PINII TERM + LOXP + SB-UBI PRO::SB- |
| | | | UBI INTRON1::ZSGREEN1::OS-UBI TERM + |
| | | | SI-ALS PRO::SI-ALS 5 UTR::ZM-ALS::SB-UBI |
| | | | TERM + LB |
| 246 | DNA | PHP93937 | RB + LOXP + ZM-EXP11232.1 PRO::ZM- |
| | | | WUS2::IN2 TERM + ZM-EXP13262 PRO::ZM- |
| | | | MPKL-A::ZM MIRNA PRECURSOR 396H::ZM- |
| | | | MPKL-A STAR SEQ::ZM-EXP23070 TERM + |
| | | | LOXP + SB-UBI PRO::SB-UBI |
| | | | INTRON1::ZSGREEN1::OS-UBI TERM + SB- |
| | | | ALS PRO::ZM-ALS::SB-UBI TERM + LB |
| 247 | DNA | PHP94638 | RB + LOXP + ZM-HSP17.7 PRO::MO-CRE |
| | | | EXON1:: ST-LS1 INTRON1::MO-CRE |
| | | | EXON2::PINII TERM + IN2 TERM::ZM- |
| | | | WUS2::ZM-UBI1 INTRON1::ZM-UBI1 |
| | | | MINPRO::ZM-UBI1 5UTR + UBI1ZM |
| | | | PRO::UBI1ZM 5UTR:: UBI1ZM INTRON1::ZM- |
| | | | ODP2::ZM-UBI TERM + LOXP + SB-UBI |
| | | | PRO::SB-UBI INTRON1::ZM-MPKL-A::ZM |
| | | | MiRNA PRECURSOR 396H::ZM-MPKL-A |
| | | | STAR SEQ::SB-GKAF TERM + LB |
| 248 | DNA | PHP98567 | RB + LOXP + UBI1ZM PRO (3X ZM-AS2 |
| | | | EME)::UBI1ZM INTRON1::ZM-WUS2::IN2 |
| | | | TERM + UBI1ZM PRO (3X ZM-AS2 |
| | | | EME)::UBI1ZM INTRON1::ZM-ODP2::OS-T28 |
| | | | TERM + CZ19B1 TERM + ZM-HSP17.7 |
| | | | PRO::MO-CRE EXON1:: ST-LS1 |
| | | | INTRON1::MO-CRE EXON2::PINII TERM + |
| | | | LOXP + SB-UBI PRO::SB-UBI |
| | | | INTRON1::ZSGREEN1::OS-UBI TERM + SB- |
| | | | ALS PRO::ZM-ALS::SB-UBI TERM + LB |
| 249 | DNA | PHP97452 | RB + LOXP + UBI1ZM PRO (3X ZM-AS2 |
| | | | EME)::UBI1ZM INTRON1::ZM-WUS2::IN2 |
| | | | TERM + UBI1ZM PRO::UBI1ZM |
| | | | 5UTR::UBI1ZM INTRON1::ZM-ODP2::OS-T28 |
| | | | TERM + CZ19B1 TERM + ZM-HSP17.7 |
| | | | PRO::MO-CRE EXON1::ST-LS1 |
| | | | INTRON1::MO-CRE EXON2::PINII TERM + |
| | | | LOXP + SB-UBI PRO::SB-UBI |
| | | | INTRON1::ZSGREEN1::OS-UBI TERM + SB- |
| | | | ALS PRO::ZM-ALS::SB-UBI TERM + LB |
| 250 | DNA | PHP97456 | RB + LOXP + UBI1ZM PRO (3X ZM-AS2 |
| | | | EME)::UBI1ZM INTRON1::ZM-WUS2::IN2 |
| | | | TERM + UBI1ZM PRO::UBI1ZM 5UTR::ADH1 |
| | | | INTRON1::ZM-ODP2::OS-T28 TERM + |
| | | | CZ19B1 TERM + ZM-HSP17.7 PRO::MO-CRE |
| | | | EXON1:: ST-LS1 INTRON1::MO-CRE |
| | | | EXON2::PINII TERM + LOXP + SB-UBI |
| | | | PRO::SB-UBI INTRON1::ZSGREEN1::OS-UBI |
| | | | TERM + SB-ALS PRO::ZM-ALS::SB-UBI |
| | | | TERM + LB |
| 251 | DNA | PHP97977 | RB + LOXP + UBI1ZM PRO (3X ZM-AS2 |
| | | | EME)::UBI1ZM INTRON1::ZM-WUS2::IN2 |
| | | | TERM + UBI1ZM PRO::UBI1ZM |
| | | | 5UTR(TR1)::ZM-ODP2::OS-T28 TERM + |
| | | | CZ19B1 TERM + ZM-HSP17.7 PRO::MO-CRE |
| | | | EXON1:: ST-LS1 INTRON1::MO-CRE |
| | | | EXON2::PINII TERM + LOXP + SB-UBI |
| | | | PRO::SB-UBI INTRON1:: ZSGREEN1::OS-UBI |
| | | | TERM + SB-ALS PRO::ZM-ALS::SB-UBI |
| | | | TERM + LB |
| 252 | DNA | PHP97449 | RB + LOXP + UBI1ZM PRO::UBI1ZM |
| | | | 5UTR(TR1)::ZM-WUS2::IN2 TERM + UBI1ZM |
| | | | PRO (3X ZM-AS2 EME)::UBI1ZM |
| | | | INTRON1::ZM-ODP2::OS-T28 TERM + |
| | | | CZ19B1 TERM + ZM-HSP17.7 PRO::MO-CRE |
| | | | EXON1::ST-LS1 INTRON1::MO-CRE EXON2:: |
| | | | PINII TERM + LOXP + SB-UBI PRO::SB-UBI |
| | | | INTRON1::ZSGREEN1::OS-UBI TERM + SB- |
| | | | ALS PRO::ZM-ALS::SB-UBI TERM + LB |
| 253 | DNA | PHP98680 | RB + FMV ENHANCER + PCSV ENH + MMV |
| | | | ENH + UBI1ZM PRO::UBI1ZM 5UTR::UBI1ZM |
| | | | INTRON1::LOXP::ZM-ODP2::TAV-T2A::ZM- |
| | | | WUS2::IN2 TERM + AT-5-IV-2 INS + ZM- |
| | | | HSP17.7 PRO::MO-CRE EXON1:: ST-LS1 |
| | | | INTRON1::MO-CRE EXON2:: PINII TERM + |
| | | | AT-5-IV-2 INS + LOXP + ZM-MIR 156B + SB- |
| | | | GKAF TERM + SB-UBI PRO::SB-UBI |
| | | | INTRON1:: ZSGREEN1::OS-UBI TERM + SI- |
| | | | ALS PRO::SI-ALS 5 UTR::ZM-ALS::SB-UBI |
| | | | TERM + LB |
| 254 | DNA | PHP98681 | RB + UBI1ZM PRO::UBI1ZM 5UTR::UBI1ZM |
| | | | INTRON1::LOXP::NOS PRO:: ZM-WUS2::IN2 |
| | | | TERM + FMV ENHANCER + PCSV ENH + |
| | | | MMV ENH + UBI1ZM PRO::UBI1ZM |
| | | | 5UTR::UBI1ZM INTRON1::ZM-ODP2::OS-T28 |
| | | | TERM + AT-5-IV-2 INS + ZM-HSP17.7 |
| | | | PRO::MO-CRE EXON1:: ST-LS1 |
| | | | INTRON1::MO-CRE EXON2:: PINII TERM + |
| | | | AT-5-IV-2 INS + LOXP + ZM-MIR 156B + SB- |
| | | | GKAF TERM + SB-UBI PRO::SB-UBI |
| | | | INTRON1:: ZSGREEN1::OS-UBI TERM + SI- |
| | | | ALS PRO::SI-ALS 5 UTR::ZM-ALS::SB-UBI |
| | | | TERM + LB |
| 255 | DNA | PHP98328 | RB + LOXP + BD-CAB2 UARI::OC EME1::OC |
| | | | EME1::BD-CAB2 PRO::ZM-HPLV9 |
| | | | INTRON1::ZM-WUS2::PINII TERM + UBI1ZM |
| | | | PRO::UBI1ZM 5UTR::UBI1ZM INTRON1::ZM- |
| | | | ODP2::PINII TERM + ZM-HSP17.7 PRO::MO- |
| | | | CRE EXON1:: ST-LS1 INTRON1::MO-CRE |
| | | | EXON2:: PINII TERM + LOXP + SB-UBI3 |
| | | | PRO::SB-UBI3 INTRON1:: ZSGREEN1::PINII |
| | | | TERM + SB-ALS PRO:: ZM-ALS::SB-PEPC1 |
| | | | TERM + LB |
| 256 | DNA | PHP98329 | RB + LOXP + ZM-GOS2 PRO (UAR):: OC |
| | | | EME1::OC EME1::ZM-GOS2 PRO (CORE)::ZM- |
| | | | GOS2 INTRON1::ZM-WUS2::PINII TERM + |
| | | | UBI1ZM PRO::UBI1ZM 5UTR::UBI1ZM |
| | | | INTRON1::ZM-ODP2::PINII TERM + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO:: ZM-ALS::SB-PEPC1 TERM + |
| | | | LB |
| 257 | DNA | PHP98327 | RB + LOXP + BD-CAB2 UARI::OC EME1::OC |
| | | | EME1::BD-CAB2 PRO::ZM-HPLV9 |
| | | | INTRON1::ZM-WUS2::PINII TERM + FMV |
| | | | ENHANCER + PCSV ENH + MMV ENH + |
| | | | UBI1ZM PRO::UBI1ZM 5UTR::UBI1ZM |
| | | | INTRON1::ZM-ODP2::OS-T28 TERM + AT-5- |
| | | | IV-7 INS + ZM-HSP17.7 PRO::MO-CRE |
| | | | EXON1:: ST-LS1 INTRON1::MO-CRE EXON2:: |
| | | | PINII TERM + OS-UBI TERM + LOXP + SI- |
| | | | UBI3 PRO::SI-UBI3 INTRON1:: |
| | | | ZSGREEN1::PINII TERM + SB-ALS PRO:: ZM- |
| | | | ALS::SB-PEPC1 TERM + LB |
| 258 | DNA | PHP98370 | RB + LOXP + ZM-GOS2 PRO (UAR):: OC |
| | | | EME1::OC EME1::ZM-GOS2 PRO (CORE)::ZM- |
| | | | GOS2 INTRON1::ZM-WUS2::PINII TERM + |
| | | | FMV ENHANCER + PCSV ENH + MMV ENH + |
| | | | UBI1ZM PRO::UBI1ZM 5UTR::UBI1ZM |
| | | | INTRON1::ZM-ODP2::OS-T28 TERM + AT-5- |
| | | | IV-7 INS + ZM-HSP17.7 PRO::MO-CRE |
| | | | EXON1:: ST-LS1 INTRON1::MO-CRE EXON2:: |
| | | | PINII TERM + OS-UBI TERM + LOXP + SI- |
| | | | UBI3 PRO::SI-UBI3 INTRON1:: |
| | | | ZSGREEN1::PINII TERM + SB-ALS PRO:: ZM- |
| | | | ALS::SB-PEPC1 TERM + LB |
| 259 | DNA | PHP98564 | RB + LOXP + ZM-PLTP PRO::ZM-PLTP |
| | | | 5UTR::ZM-WUS2::IN2 TERM + UBI1ZM |
| | | | PRO::UBI1ZM 5UTR::UBI1ZM INTRON1::ZM- |
| | | | ODP2::PINII TERM + ZM-HSP17.7 PRO::MO- |
| | | | CRE EXON1:: ST-LS1 INTRON1::MO-CRE |
| | | | EXON2:: PINII TERM + OS-UBI TERM + |
| | | | LOXP + SB-UBI3 PRO::SB-UBI3 INTRON1:: |
| | | | ZSGREEN1::PINII TERM + SB-ALS PRO:: ZM- |
| | | | ALS::SB-PEPC1 TERM + LB |
| 260 | DNA | PHP98565 | RB + LOXP + ZM-PLTP PRO::ZM-PLTP |
| | | | 5UTR::ZM-WUS2::IN2 TERM + FMV |
| | | | ENHANCER + PCSV ENH + MMV ENH + |
| | | | UBI1ZM PRO::UBI1ZM 5UTR::UBI1ZM |
| | | | INTRON1::ZM-ODP2::OS-T28 TERM + AT-5- |
| | | | IV-7 INS + ZM-HSP17.7 PRO::MO-CRE |
| | | | EXON1:: ST-LS1 INTRON1::MO-CRE EXON2:: |
| | | | PINII TERM + OS-UBI TERM + LOXP + SI- |
| | | | UBI3 PRO::SI-UBI3 INTRON1:: |
| | | | ZSGREEN1::PINII TERM + SB-ALS PRO:: ZM- |
| | | | ALS::SB-PEPC1 TERM + LB |
| 261 | DNA | PHP97447 | RB + LOXP + ZM-HSP17.7 PRO::MO-CRE |
| | | | EXON1:: ST-LS1 INTRON1::MO-CRE EXON2:: |
| | | | PINII TERM + UBI1ZM PRO::UBI1ZM |
| | | | 5UTR::UBI1ZM INTRON1::ZM-WUS2::IN2 |
| | | | TERM + UBI1ZM PRO (3X ZM-AS2 EME):: |
| | | | UBI1ZM INTRON1::ZM-ODP2::OS-T28 TERM + |
| | | | LOXP + SB-UBI PRO::SB-UBI INTRON1:: |
| | | | ZSGREEN1::OS-UBI TERM + SI-ALS PRO::SI- |
| | | | ALS 5UTR::ZM-ALS::SB-UBI TERM + LB |
| 262 | DNA | PHP97881 | RB + LOXP + UBI1ZM PRO::UBI1ZM |
| | | | 5UTR::UBI1ZM INTRON1::ZM-WUS2::IN2 |
| | | | TERM + UBI1ZM PRO (3X ZM-AS2 EME):: |
| | | | UBI1ZM INTRON1::ZM-ODP2::OS-T28 TERM + |
| | | | ZM-HSP17.7 PRO::MO-CRE EXON1:: ST-LS1 |
| | | | INTRON1::MO-CRE EXON2:: PINII TERM + |
| | | | LOXP + SB-UBI PRO::SB-UBI INTRON1:: |
| | | | ZSGREEN1::OS-UBI TERM + SI-ALS PRO::SI- |
| | | | ALS 5UTR::ZM-ALS::SB-UBI TERM + LB |
| 263 | DNA | PHP99676 | RB + LOXP + NOS PRO::ZM-WUS2::PINII |
| | | | TERM + ZM-GRP1 PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 264 | DNA | PHP99677 | RB + LOXP + NOS PRO::ZM-WUS2::PINII |
| | | | TERM + ZM-RPL1 PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 265 | DNA | PHP99678 | RB + LOXP + NOS PRO::ZM-WUS2::PINII |
| | | | TERM + ZM-DNAJ PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 266 | DNA | PHP99679 | RB + LOXP + NOS PRO::ZM-WUS2::PINII |
| | | | TERM + ZM-SAMDC2 PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 267 | DNA | PHP99680 | RB + LOXP + NOS PRO::ZM-WUS2::PINII |
| | | | TERM + ZM-PPISO PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 268 | DNA | PHP99569 | RB + LOXP + NOS PRO::ZM-WUS2::PINII |
| | | | TERM + ZM-EF1A PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 269 | DNA | PHP100011 | RB + LOXP + NOS PRO::ZM-WUS2::PINII |
| | | | TERM + FMV ENHANCER + PCSV ENH + |
| | | | MMV ENH + ZM-RPL1 PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 270 | DNA | PHP100012 | RB + LOXP + NOS PRO::ZM-WUS2::PINII |
| | | | TERM + FMV ENHANCER + PCSV ENH + |
| | | | MMV ENH + ZM-DNAJ PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 271 | DNA | PHP100013 | RB + LOXP + NOS PRO::ZM-WUS2::PINII |
| | | | TERM + FMV ENHANCER + PCSV ENH + |
| | | | MMV ENH + ZM-SAMDC2 PRO::ZM- |
| | | | ODP2::PINII TERM + AT-5-IV-7 INS + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 272 | DNA | PHP100056 | RB + LOXP + NOS PRO::ZM-WUS2::PINII |
| | | | TERM + FMV ENHANCER + PCSV ENH + |
| | | | MMV ENH + ZM-PPISO PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 273 | DNA | PHP100057 | RB + LOXP + NOS PRO::ZM-WUS2::PINII |
| | | | TERM + FMV ENHANCER + PCSV ENH + |
| | | | MMV ENH + ZM-EF1A PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 274 | DNA | PHP100158 | RB + LOXP + NOS PRO::ZM-WUS2::PINII |
| | | | TERM + FMV ENHANCER + PCSV ENH + |
| | | | MMV ENH + ZM-GRP1 PRO::ZM-ODP2::PINII |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SI-UBI3 PRO::SI- |
| | | | UBI3 INTRON1::ZSGREEN1::PINII TERM + |
| | | | SB-ALS PRO::ZM-ALS::SB-PEPC1 TERM + LB |
| 275 | DNA | PHP98229 | RB + LOXP + NOS PRO::ZM-WUS2::IN2 |
| | | | TERM + AT-5-IV-2 INS + FMV ENHANCER + |
| | | | PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-2 INS + ZM- |
| | | | HSP17.7 PRO::MO-CRE EXON1:: ST-LS1 |
| | | | INTRON1::MO-CRE EXON2:: PINII TERM + |
| | | | LOXP + SB-UBI PRO::SB-UBI INTRON1:: |
| | | | ZSGREEN1::OS-UBI TERM + SI-ALS PRO::SI- |
| | | | ALS 5 UTR::ZM-ALS::SB-UBI TERM + LB |
| 276 | DNA | PHP100159 | RB + LOXP + NOS PRO::ZM-WUS2::IN2 |
| | | | TERM + FMV ENHANCER + PCSV ENH + |
| | | | MMV ENH + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::ZM-CYCD2::SB- |
| | | | GKAF TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1::ZSGREEN1::PINII TERM + |
| | | | SB-ALS PRO:: ZM-ALS::SB-PEPC1 TERM + LB |
| 277 | DNA | PHP100160 | RB + LOXP + NOS PRO::ZM-WUS2::IN2 |
| | | | TERM + FMV ENHANCER + PCSV ENH + |
| | | | MMV ENH + UBI1ZM PRO::UBI1ZM |
| | | | 5UTR::UBI1ZM INTRON1::ZM-ODP2::OS-T28 |
| | | | TERM + UBI1ZM PRO::UBI1ZM |
| | | | 5UTR::UBI1ZM INTRON1::REPA(WDV)::SB- |
| | | | GKAF TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 278 | DNA | PHP100229 | RB + LOXP + ZM-ATP SYN PRO::ZM- |
| | | | WUS2::PINII TERM + FMV ENHANCER + |
| | | | PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-7 INS + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 279 | DNA | PHP99971 | RB + LOXP + ZM-EF4A PRO::ZM-WUS2::PINII |
| | | | TERM + FMV ENHANCER + PCSV ENH + |
| | | | MMV ENH + UBI1ZM PRO::UBI1ZM |
| | | | 5UTR::UBI1ZM INTRON1::ZM-ODP2::OS-T28 |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 280 | DNA | PHP99809 | RB + LOXP + ZM-PABP PRO::ZM- |
| | | | WUS2::PINII TERM + FMV ENHANCER + |
| | | | PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-7 INS + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 281 | DNA | PHP99810 | RB + LOXP + ZM-VDACIA PRO::ZM- |
| | | | WUS2::PINII TERM + FMV ENHANCER + |
| | | | PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-7 INS + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 282 | DNA | PHP99716 | RB + LOXP + ZM-LEA14 PRO::ZM- |
| | | | WUS2::PINII TERM + FMV ENHANCER + |
| | | | PCSV ENH + MMV ENH + UBI1ZM |
| | | | PRO::UBI1ZM 5UTR::UBI1ZM INTRON1::ZM- |
| | | | ODP2::OS-T28 TERM + AT-5-IV-7 INS + ZM- |
| | | | HSP17.7 PRO::MO-CRE EXON1:: ST-LS1 |
| | | | INTRON1::MO-CRE EXON2:: PINII TERM + |
| | | | OS-UBI TERM + LOXP + SB-UBI3 PRO::SB- |
| | | | UBI3 INTRON1:: ZSGREEN1::PINII TERM + |
| | | | SB-ALS PRO::ZM-ALS::SB-PEPC1 TERM + LB |
| 283 | DNA | PHP99721 | RB + LOXP + NOS::WUS2::IN2 TERM + FMV |
| | | | ENHANCER + PCSV ENH + MMV ENH + |
| | | | UBI1ZM PRO::UBI1ZM 5UTR::UBI1ZM |
| | | | INTRON1::ZM-ODP2::OS-T28 TERM + AT-5- |
| | | | IV-7 INS + HSP17.7 PRO::MO-CRE::PINII |
| | | | TERM + UBI1ZM PRO::CAS9::ZM-UBI TERM + |
| | | | ZM-U6 PRO::gRNA-CHR1-53.66 + ZM-ALS |
| | | | PRO::HRA::SB-UBI TERM + CHR1-53.66 |
| | | | TARGET SITE + HOMOLOGY SEQ1 + SI-UBI |
| | | | PRO::NPTII::SI-UBI TERM + HOMOLOGY |
| | | | SEQ2 + CHR1-53.66 TARGET SITE + SB-UBI |
| | | | PRO::ZS-GREEN1::OS-UBI TERM + LB |
| 284 | DNA | PHP97978 | RB + LOXP + NOS::WUS2::IN2 TERM + |
| | | | UBI1ZM PRO::UBI1ZM 5UTR::UBI1ZM |
| | | | INTRON1::ZM-ODP2::OS-T28 TERM + |
| | | | CZ19B1 TERM + HSP17.7 PRO::MO- |
| | | | CRE::PINII TERM + LOXP + SB-UBI PRO::SB- |
| | | | UBI INTRON1:: ZSGREEN1::OS-UBI TERM + |
| | | | SB-ALS PRO::ZM-ALS::SB-UBI TERM + LB |
| 285 | DNA | PHP101270 | RB + LOXP + NOS::WUS2::IN2 TERM + SCBV |
| | | | PRO::SCBV 5UTR::ZM-ODP2::PINII TERM + |
| | | | AT-5-IV-7 INS + ZM-HSP17.7 PRO::MO-CRE |
| | | | EXON1::ST-LS1 INTRON1::MO-CRE EXON2:: |
| | | | PINII TERM + OS-UBI TERM + LOXP + SB- |
| | | | UBI3 PRO::SB-UBI3 INTRON1:: |
| | | | ZSGREEN1::PINII TERM + SB-ALS PRO::ZM- |
| | | | ALS::SB-PEPC1 TERM + LB |
| 286 | DNA | PHP102481 | RB + LOXP + NOS::WUS2::IN2 TERM + BD- |
| | | | CAB2 UAR1::OC EME 1::OC EME 1::BD-CAB2 |
| | | | PRO::ZM-HPLV9 INTRON::ZM-ODP2::OS-T28 |
| | | | TERM + AT-5-IV-7 INS + ZM-HSP17.7 |
| | | | PRO::MO-CRE EXON1:: ST-LS1 |
| | | | INTRON1::MO-CRE EXON2:: PINII TERM + |
| | | | OS-UBI TERM + LOXP + SB-UBI PRO::SB-UBI |
| | | | INTRON1:: ZSGREEN1::OS-UBI TERM + SI- |
| | | | UBII PRO::SI-UBI1 INTRON::NPTII::SB-UBI |
| | | | TERM + LB |
| 287 | DNA | BD-UBI1 | Promoter for Ubiquitin 1 from Brachypodium |
| | | | distachyon |
| 288 | DNA | BD-UBIIC | Promoter for Ubiquitin 1C from Brachypodium |
| | | | distachyon |
| 289 | DNA | BSV(AY) | Promoter from Banana streak virus |
| 290 | DNA | AT-NOS | Nopaline Synthase promoter from Agrobacterium |
| | | PRO | tumefaciens |
| 291 | DNA | ZM-AXIG1 | Zea mays promoter upregulated by banveland |
| | | PRO | auxin |
| 292 | DNA | ZM-PLTP | Zea mays promoter from phospholipid transfer |
| | | PRO | protein (PLTP) homolog |
| 293 | DNA | 35S | Cauliflower mosaic virus enhancer element |
| | | Enhancer | |
| 294 | DNA | ZM-CAB | Zea mays cab-1 gene for chlorophyll a/b-binding |
| | | PRO | protein |
| 295 | DNA | SCBV PRO | Promoter from an Australian isolate of Sugarcane |
| | | | bacilliform virus |
| 296 | DNA | 8xDR5 PRO | Synthetic promoter |
| 297 | DNA | FT-MEM1 | Flaveria trinervia transciption factor binding site |
| 298 | DNA | ZM-PEPC1 | Zea mays Phospoenolpyruvate Carboxylase1 |
| | | PRO | promoter |
| 299 | DNA | DIURNAL12 | Zea mays thiazole biosynthetic enzyme 1-1, |
| | | Promoter | chloroplast promoter |
| 300 | DNA | RUBISCO | Zea mays promoter for the Ribulose bisphosphate |
| | | SSU PROr | carboxylase (RUBISCO) Small Subunit protein |
| 301 | DNA | CSVMV | Cassava vein mosaic virus promoter |
| | | PRO | |
| 302 | DNA | FT-PPCA1 | Flaveria trinervia Phosphoenolpyruvate |
| | | PRO | carboxylase A1 promoter |
| 303 | DNA | ZM-GOS2 | Zea mays GOS2 promoter |
| | | PRO | |
| 304 | DNA | ZM- | Zea mays SWEET11 promoter |
| | | SWEET11 | |
| | | PRO | |
| 305 | DNA | ZM- | Zea mays diurnal promoter #10 |
| | | DIURNAL10 | |
| | | PRO | |
| 306 | DNA | ZM- | Zea mays diurnal promoter #11 |
| | | DIURNAL11 | |
| | | PRO | |
| 307 | DNA | ZM-ADH | Zea mays alcohol dehydrogenase intron1 |
| | | INTRON | |
| 308 | DNA | ZM-LEC1 | Zea mays LEC1 (Leafy cotyledon 1) gene |
| 309 | PRT | ZM-LEC1 | Encoded protein of the Zea mays LEC1 gene |
| 310 | DNA | PLTP2 PPO | Zea mays promoter from phospholipid transfer |
| | | | protein2 (PLTP2) |
| 311 | DNA | REPA | Wheat dwarf virus REPA gene |
| 312 | PRT | REPA | Encoded protein of the Wheat dwarf virus REPA |
| | | | gene |
| 313 | DNA | amiPKL-A | Synthetic artificial micro-RNA that targets the Zea |
| | | | mays PICKLE (PKL) transcript. |
| 314 | DNA | ZM- | Full-length Zea mays Grain Protein Content NAC |
| | | GPCNAC-1 | transcription factor gene |
| 315 | PRT | ZM- | Encoded protein for the Zea mays Grain Protein |
| | | GPCNAC-1 | Content NAC transcription factor gene |
| 316 | DNA | RKD4 | Maize-optimized version of the Arabidopsis RWP- |
| | | | RK-type transcription factor |
| 317 | PRT | AT-RKD4 | Encoded protein for the maize-optimized version |
| | | | of the Arabidopsis RWP-RK-type transcription |
| | | | factor |
| 318 | DNA | MO-LEC2 | Maize codon-optimized version of the Arabidopsis |
| | | | thaliana AtLEC2 LEAFY COTYLEDON 2 gene |
| 319 | PRT | AT-LEC2 | Encoded protein of the maize codon-optimized |
| | | | version of the Arabidopsis thaliana LEC2 LEAFY |
| | | | COTYLEDON 2 gene |
| 320 | DNA | RAP2.6L | Maize codon-optimized version of the Arabidopsis |
| | | | thaliana RAP2.6L gene |
| 321 | PRT | AT-RAP2.6L | Encoded protein of the maize codon-optimized |
| | | | version of the Arabidopsis thaliana RAP2.6L gene |
| 322 | DNA | AT-5-IV-2 | Sequence from Arabidopsis thaliana with insulator |
| | | | properties |
| 323 | DNA | ZM-RLD1 | Zea mays Rolled Leaf 1 homolog of Revoluta |
| | | | from Arabidopsis thaliana |
| 324 | PRT | ZM-RLD1 | Encoded protein of the Zea mays Rolled Leaf 1 |
| | | | homolog of Revoluta from Arabidopsis thaliana |
| 325 | DNA | ZM-KN1 | Zea mays class I homeobox transcription factor |
| | | | Knotted1 gene |
| 326 | PRT | KN1 | Encoded protein of the Zea mays class I homeobox |
| | | | transcription factor Knotted1 gene |
| 327 | DNA | AT-5-IV-7 | Sequence from Arabidopsis with insulator |
| | | | properties |
| 328 | DNA | ZM-CYCD2 | Zea mays Cyclin delta-2 gene |
| 329 | PRT | ZM-CYCD2 | Encoded protein of the Zea mays Cyclin delta-2 |
| | | | gene |
| 330 | DNA | HSP17.7 | Zea mays promoter for Heat Shock Protein 17.7 |
| | | PRO | |
| 331 | DNA | RAB 17 PRO | Zea mays promoter for RAB17 |
| 332 | DNA | GLB1 PRO | Zea mays globulin 1 promoter |
| 333 | DNA | ZM-UBI1 | Zea mays ubiquitin1 promoter |
| | | PRO | |
| | | (UBI1ZM | |
| | | PRO) | |
| 334 | DNA | ZM-UBI1 | Zea mays ubiquitin1 5′ untranslated region |
| | | 5UTR | |
| | | (UBI1ZM | |
| | | 5UTR) | |
| 335 | DNA | ZM-UBI1 | Zea mays ubiquitin1 intron1 |
| | | INTRON1 | |
| | | (UBI1ZM | |
| | | INTRON1) | |
| 336 | DNA | FMV ENH | Figwort mosaic virus enhancer |
| 337 | DNA | PCSV ENH | Peanut Chlorotic Streak Caulimovirus enhancer |
| 338 | DNA | MMV ENH | Mirabilis mosaic virus enhancer |
| 339 | DNA | ZM-UBI1 | Zea mays ubiquitin1 promoter (SEQ ID NO: 333) + |
| | | PRO | Zea mays ubiquitin1 intron1 (SEQ ID NO: 334) + |
| | | Complete | Zea mays ubiquitin1 intron1 (SEQ ID NO: 335) |
| 340 | DNA | 3xENH | Figwort mosaic virus enhancer (SEQ ID NO: 336) + |
| | | | Peanut Chlorotic Streak Caulimovirus enhancer |
| | | | (SEQ ID NO: 337) + Mirabilis Mosaic Virus |
| | | | enhancer (SEQ ID NO: 338) |
| 341 | DNA | PHP40828 | NOS PRO::ZM-WUS2::PINII TERM |
| 342 | DNA | PHP100712 | RB + LOXP + UBI1ZM PRO::UBI1ZM |
| | | | 5UTR::UBI1ZM INTRON1::WUS2::IN2 TERM + |
| | | | FMV ENHANCER + PCSV ENH + MMV ENH + |
| | | | SI-UBII PRO::SI-UBI1 INTRON1::ZM- |
| | | | ODP2::ZM-UBI TERM TERM + LOXP + NOS |
| | | | PRO::CRC::SB-GKAF TERM + LB |
| 343 | DNA | PHV00001 | RB + LOXP + FMV ENH:PSCV ENH:MMV |
| | | | ENH:UBI1ZM PRO::UBI1ZM 5UTR:UBI1ZM |
| | | | INTRON1::ZM-ODP2::OS-T28 TERM + |
| | | | HSP17.7 PRO::CRE EXON1:ST-LS1 |
| | | | INTRON2:CRE EXON2::PINII TERM + LOXP + |
| | | | SB-UBI PRO::SB-UBI INTRON1::ZS- |
| | | | GREEN::OS-UBI TERM + SI-UBI PRO::SI-UBI |
| | | | INTRON1::NPTII::SB-UBI TERM + LB |
| 344 | DNA | PHV00002 | RB + LOXP + FMV ENH:PSCV ENH:MMV |
| | | | ENH:UBI1ZM PRO::UBI1ZM 5UTR:UBI1ZM |
| | | | INTRON1::ZM-WUS2::OS-T28 TERM + |
| | | | HSP17.7 PRO::CRE EXON1:ST-LS1 |
| | | | INTRON2:CRE EXON2::PINII TERM + LOXP + |
| | | | SB-UBI PRO::SB-UBI INTRON1::ZS- |
| | | | GREEN::OS-UBI TERM + SI-UBI PRO::SI-UBI |
| | | | INTRON1::NPTII::SB-UBI TERM + LB |
| 345 | DNA | PHV00003 | RB + LOXP + ZM-GOS2 PRO::SB-UBI |
| | | | INTRON1::MO-LEXA:MO-CBF1A::SB-ACTIN |
| | | | TERM + 6xREC:MIN35S PRO::ZM-ODP2::OS- |
| | | | UBI TERM + HSP17.7 PRO::CRE EXON1:ST- |
| | | | LS1 INTRON2:CRE EXON2::PINII TERM + |
| | | | LOXP + SB-UBI PRO::SB-UBI INTRON1::ZS- |
| | | | GREEN::OS-UBI TERM + SI-UBI PRO::SI-UBI |
| | | | INTRON1::NPTII::SB-UBI TERM + LB |
| 346 | DNA | PHV00004 | RB + LOXP + ZM-GOS2 PRO::SB-UBI |
| | | | INTRON1::MO-LEXA:MO-CBF1A::SB-ACTIN |
| | | | TERM + 6xREC:MIN35S PRO::ZM-WUS2::OS- |
| | | | UBI TERM + HSP17.7 PRO::CRE EXON1:ST- |
| | | | LS1 INTRON2:CRE EXON2::PINII TERM + |
| | | | LOXP + SB-UBI PRO::SB-UBI INTRON1::ZS- |
| | | | GREEN::OS-UBI TERM + SI-UBI PRO::SI-UBI |
| | | | INTRON1::NPTII::SB-UBI TERM + LB |
| 347 | DNA | 3xENH-UBI | 3xENH-UBI |
| 348 | DNA | MO-LEXA- | Fusion protein coding sequence composed of the |
| | | MO-CBF1A | maize-optimized LEXA gene via a flexible protein |
| | | | linker peptide to a maize-optimized CBF1A gene |
| 349 | PRT | MO-LEXA- | Encoded fusion protein composed of the maize- |
| | | MO-CBF1A | optimized LEXA DNA-binding polypeptide, a |
| | | PRT | flexible protein linker peptide, and the CBF1A |
| | | | activation domain |
| 350 | DNA | 6xREC- | Six repeats of the LEXA binding motif plus the 45 |
| | | MIN35S | base-pair minimal 35S promoter core |
| 351 | DNA | PHP101093 | RB + LOXP + NOS::ZM-WUS2::IN2 TERM + |
| | | | FMV ENHANCER + PCSV ENH + MMV ENH + |
| | | | UBI1ZM PRO::UBI1ZM 5UTR::UBI1ZM |
| | | | INTRON1::ZM-ODP2::OS-T28 TERM + AT-5- |
| | | | IV-7 INS + HSP17.7 PRO::MO-CRE::PINII |
| | | | TERM + OS-UBI TERM + LOXP + SI-UBI3 |
| | | | PRO:: SI-UBI3 INTRON1:: ZS-GREEN1::OS- |
| | | | UBI TERM + SI-UBI1 PRO::SI-UBI1 |
| | | | INTRON1::NPTII (MO)::SB-UBI TERM + LB |
| 352 | DNA | PHP104215 | RB + LOXP + OS-ACTIN PRO::OS-ACTIN |
| | | | INTRON1::ZM-WUS2::IN2 TERM + GZ-W64A |
| | | | TERM + FL TERM + FMV ENHANCER + PCSV |
| | | | ENH + MMV ENH + UBI1ZM PRO::UBI1ZM |
| | | | 5UTR::UBI1ZM INTRON1::ZM-ODP2::OS-T28 |
| | | | TERM + AT-5-IV-7 INS + RAB17 PRO::RAB17 |
| | | | 5UTR::MO-CRE::PINII TERM + OS-UBI TERM + |
| | | | LOXP + SB-UBI PRO:: SB-UBIINTRON1:: ZS- |
| | | | GREEN1::OS-UBI TERM + SI-UBII PRO::SI- |
| | | | UBI1 INTRON1::NPTII (MO)::SB-UBI TERM + |
| | | | LB |
| 353 | DNA | PHP104222 | RB + LOXP + OS-ACTIN PRO::OS-ACTIN |
| | | | INTRON1::ZM-WUS2::IN2 TERM + GZ-W64A |
| | | | TERM + FL TERM + FMV ENHANCER + PCSV |
| | | | ENH + MMV ENH + UBI1ZM PRO::UBI1ZM |
| | | | 5UTR::UBI1ZM INTRON1::ZM-ODP2::OS-T28 |
| | | | TERM + AT-5-IV-7 INS + ZM-HSP17.7 |
| | | | PRO::MO-CRE::PINII TERM + OS-UBI TERM + |
| | | | LOXP + SB-UBI PRO:: SB-UBI INTRON1:: ZS- |
| | | | GREEN1::OS-UBI TERM + SI-UBII PRO::SI- |
| | | | UBI1 INTRON1::NPTII (MO)::SB - UBI TERM + |
| | | | LB |

### Example 2: Media Compositions

Various media are referenced in the Examples for use in transformation and cell culture. The compositions of these media are provided below in Tables 3-14.

TABLE 3
Medium Composition

PHI-I: 4.3 g/l MS salts (Phytotechnology Laboratories, Shawnee Mission, KS, catalog
number M524), 0.5 mg/l nicotinic acid, 0.5 mg/l pyridoxine HCl, 1 mg/l thiamine HCl,
0.1 g/l myo-inositol, 1 g/l casamino acids (Becton Dickinson and Company, BD
Diagnostic Systems, Sparks, MD, catalog number 223050), 1.5 mg/l 2,4-
dichlorophenoxyacetic acid (2,4-D), 68.5 g/l sucrose, 36 g/l glucose, pH 5.2; with
100 μM acetosyringone added before using.
PHI-T: PHI-I with 20 g/l sucrose, 10 g/l glucose, 2 mg/l 2,4-D, no casamino acids,
0.5 g/l MES buffer, 0.7 g/l L-proline, 10 mg/l ascorbic acid, 100 μM acetosyringone,
8 g/l agar, pH 5.8.
PHI-U: PHI-T with 1.5 mg/l 2,4-D 100 mg/l carbenicillin, 30 g/l sucrose, no glucose
and acetosyringone; 5 mg/l PPT, pH 5.8.
PHI-UM: PHI-U with 12.5 g/l mannose and 5 g/l maltose, no sucrose, no PPT, pH 5.8
PHI-V: PHI-U with 10 mg/l PPT
DBC3: 4.3 g/l MS salts, 0.25 g/l myo-inositol, 1.0 g/l casein hydrolysate, 1.0 mg/l
thiamine HCL, 1.0 mg/l 2,4-D, 30 g/l maltose, 0.69 g/l L-proline, 1.22 mg/l cupric
sulfate, 0.5 mg/l BAP, 3.5 g/l phytagel, pH 5.8
PHI-X: 4.3 g/l MS salts, 0.1 g/l myo-inositol, 5.0 ml MS vitamins stock^{a}, 0.5 mg/l
zeatin, 700 mg/l L-proline, 60 g/l sucrose, 1 mg/l indole-3-acetic acid, 0.1 μM abscisic
acid, 0.1 mg/l thidiazuron, 100 mg/l carbenicillin, 5 mg/l PPT, 8 g/l agar, pH 5.6.
PHI-XM: PHI-X with no PPT; added 1.25 mg/l cupric sulfate, pH 5.6.
PHI-Z: 2.15 g/l MS salts, 0.05 g/l myo-inositol, 2.5 ml MS vitamins stock^{a}, 20 g/l
sucrose, 3 g/l phytagel, pH 5.6
^{a}MS vitamins stock: 0.1 g/l nicotinic acid, 0.1 g/l pyridoxine HCl, 0.02 g/l thiamine HCl, 0.4 g/l glycine.

TABLE 4

| WI 4 | | |
|---|---|---|
| DI water | 1000 | mL |
| MS salt + Vitamins(M519) | 4.43 | g |
| Maltose | 30 | g |
| Glucose | 10 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| Adjust PH to 5.8 with KOH | | |
| Post-sterilization add: | | |
| Acetosyringone (400 μM) | 400 | μl |

TABLE 5

| WC # 10 | | |
|---|---|---|
| DI water | 1000 | mL |
| MS salt + Vitamins(M519) | 4.43 | g |
| Maltose | 30 | g |
| Glucose | 1 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| 50X CuSO4 (.1M) | 49 | μl |
| Adjust PH to 5.8 with KOH and add 2.5 g/L of Phytagel. | | |
| Post-sterilization add: | | |
| Acetosyringone (400 μM) | 400 | μl |

TABLE 6

| DBC4 | | |
|---|---|---|
| dd H20 | 1000 | mL |
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 2 | mL |
| BAP | 1 | mL |
| Adjust PH to 5.8 with KOH and then add 3.5 g/L of Phytagel. | | |
| Post-sterilization add: | | |
| Cef (100 mg/mL) | 1 | mL |

TABLE 7
DBC6

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 1 | mL |
| BAP | 2 | mL |
| Adjust PH to 5.8 with KOH and then add 3.5 | | |
| g/L of Phytagel. | | |
| Post-sterilization add: | | |
| Cef (100 mg/mL) | 1 | mL |

TABLE 8
MSA

| dd H20 | 1000 | mL |
|---|---|---|
| MS salt + Vitamins(M519) | 4.43 | g |
| Sucorse | 20 | g |
| Myo- Inositol | 1 | g |
| Adjust PH to 5.8 with KOH and then add 3.5 | | |
| g/L of Phytagel. | | |
| Post-sterilization add: | | |
| Cef (100 mg/mL) | 1 | mL |

TABLE 9
MSB

| dd H20 | 1000 | mL |
|---|---|---|
| MS salt + Vitamins(M519) | 4.43 | g |
| Sucorse | 20 | g |
| Myo- Inositol | 1 | g |
| Adjust PH to 5.8 with KOH and then add 3.5 | | |
| g/L of Phytagel. | | |
| Post-sterilization add: | | |
| Cef (100 mg/mL) | 1 | mL |
| IBA | 0.5 | mL |

TABLE 10

| | Units per | | | | | | | |
|---|---|---|---|---|---|---|---|---|
| Medium Components | liter | 12V | 810I | 700A | 710I | 605J | 605T | 289Q |
| MS BASAL SALT | g | | | 4.3 | 4.3 | 4.3 | 4.3 | 4.3 |
| MIXTURE | | | | | | | | |
| N6 | mL | | | | | 60.0 | 60.0 | |
| MACRONUTRIENTS | | | | | | | | |
| 10X | | | | | | | | |
| POTASSIUM NITRATE | g | | | | | 1.7 | 1.7 | |
| B5H MINOR SALTS | mL | | | | | 0.6 | 0.6 | |
| 1000X | | | | | | | | |
| NaFe EDTA FOR B5H | mL | | | | | 6.0 | 6.0 | |
| 100X | | | | | | | | |
| ERIKSSON'S | mL | | | | | 0.4 | 0.4 | |
| VITAMINS 1000X | | | | | | | | |
| S&H VITAMIN STOCK | mL | | | | | 6.0 | 6.0 | |
| 100X | | | | | | | | |
| THIAMINE .HCL | mg | | | 10.0 | 10.0 | 0.5 | 0.5 | |
| L-PROLINE | g | | | | 0.7 | 2.0 | 2.0 | 0.7 |
| CASEIN | g | | | | | 0.3 | 0.3 | |
| HYDROLYSATE | | | | | | | | |
| (ACID) | | | | | | | | |
| SUCROSE | g | | | 68.5 | 20.0 | 20.0 | 20.0 | 60.0 |
| GLUCOSE | g | 5.0 | | 36.0 | 10.0 | 0.6 | 0.6 | |
| MALTOSE | g | | | | | | | |
| 2,4-D | mg | | | 1.5 | 2.0 | 0.8 | 0.8 | |
| AGAR | g | 15.0 | 15.0 | | 8.0 | 6.0 | 6.0 | 8.0 |
| PHYTAGEL | g | | | | | | | |
| DICAMBA | g | | | | | 1.2 | 1.2 | |
| SILVER NITRATE | mg | | | | | 3.4 | 3.4 | |
| AGRIBIO Carbenicillin | mg | | | | | 100.0 | | |
| Timentin | mg | | | | | | 150.0 | 150.0 |
| Cefotaxime | mg | | | | | | 100.0 | 100.0 |
| MYO-INOSITOL | g | | | 0.1 | 0.1 | | | 0.1 |
| NICOTINIC ACID | mg | | | 0.5 | 0.5 | | | |
| PYRIDOXINE.HCL | mg | | | 0.5 | 0.5 | | | |
| VITAMIN ASSAY | g | | | 1.0 | | | | |
| CASAMINO ACIDS | | | | | | | | |
| MES BUFFER | g | | | | 0.5 | | | |
| ACETOSYRINGONE | μM | | | | 100.0 | | | |
| ASCORBIC ACID | mg | | | | 10.0 | | | |
| 10 MG/ML (7S) | | | | | | | | |
| MS VITAMIN STOCK | mL | | | | | | | 5.0 |
| SOL. | | | | | | | | |
| ZEATIN | mg | | | | | | | 0.5 |
| CUPRIC SULFATE | mg | | | | | | | 1.3 |
| IAA 0.5 MG/ML (28A) | mL | | | | | | | 2.0 |
| ABA 0.1 mm | mL | | | | | | | 1.0 |
| THIDIAZURON | mg | | | | | | | 0.1 |
| AGRIBIO Carbenicillin | mg | | | | | | | 100.0 |
| PPT(GLUFOSINATE- | mg | | | | | | | |
| NH4) | | | | | | | | |
| BAP | mg | | | | | | | 1.0 |
| YEAST EXTRACT (BD | g | | 5.0 | | | | | |
| Difco) | | | | | | | | |
| PEPTONE | g | | 10.0 | | | | | |
| SODIUM CHLORIDE | g | | 5.0 | | | | | |
| SPECTINOMYCIN | mg | 50.0 | 100.0 | | | | | |
| FERROUS | mL | 2.0 | | | | | | |
| SULFATE.7H20 | | | | | | | | |
| AB BUFFER 20X (12D) | mL | 50.0 | | | | | | |
| AB SALTS 20X (12E) | mL | 50.0 | | | | | | |
| Benomyl | mg | | | | | | | |
| pH | | | | | | | | 5.6 |

TABLE 11

| | Units | | | |
|---|---|---|---|---|
| | per | | | |
| Medium Components | liter | 289R | 13158H | 13224B |
| MS BASAL SALT MIXTURE | g | 4.3 | 4.3 | |
| N6 MACRONUTRIENTS 10X | mL | | | 4.0 |
| POTASSIUM NITRATE | g | | | |
| B5H MINOR SALTS 1000X | mL | | | |
| NaFe EDTA FOR B5H 100X | mL | | | |
| ERIKSSON'S VITAMINS 1000X | mL | | | 1.0 |
| S&H VITAMIN STOCK 100X | mL | | | |
| THIAMINE•HCL | mg | | | 0.5 |
| L-PROLINE | g | 0.7 | 0.7 | 2.9 |
| CASEIN HYDROLYSATE (ACID) | g | | | |
| SUCROSE | g | 60.0 | 60.0 | 190.0 |
| GLUCOSE | g | | | |
| MALTOSE | g | | | |
| 2,4-D | mg | | | |
| AGAR | g | | 8.0 | 6.4 |
| PHYTAGEL | g | | | |
| DICAMBA | g | | | |
| SILVER NITRATE | mg | | | 8.5 |
| AGRIBIO Carbenicillin | mg | | | |
| Timentin | mg | 150.0 | 150.0 | |
| Cefotaxime | mg | 100.0 | 100.0 | 25 |
| MYO-INOSITOL | g | 0.1 | 0.1 | |
| NICOTINIC ACID | mg | | | |
| PYRIDOXINE•HCL | mg | | | |
| VITAMIN ASSAY CASAMINO | g | | | |
| ACIDS | | | | |
| MES BUFFER | g | | | |
| ACETOSYRINGONE | μM | | | |
| ASCORBIC ACID 10 MG/ML (7S) | mg | | | |
| MS VITAMIN STOCK SOL. | mL | 5.0 | 5.0 | |
| ZEATIN | mg | 0.5 | 0.5 | |
| CUPRIC SULFATE | mg | 1.3 | 1.3 | |
| IAA 0.5 MG/ML (28A) | mL | 2.0 | 2.0 | |
| ABA 0.1 mm | mL | 1.0 | 1.0 | |
| THIDIAZURON | mg | 0.1 | 0.1 | |
| AGRIBIO Carbenicillin | mg | | | |
| PPT(GLUFOSINATE-NH4) | mg | | | |
| BAP | mg | | | |
| YEAST EXTRACT (BD Difco) | g | | | |
| PEPTONE | g | | | |
| SODIUM CHLORIDE | g | | | |
| SPECTINOMYCIN | mg | | | |
| FERROUS SULFATE•7H20 | mL | | | |
| AB BUFFER 20X (12D) | mL | | | |
| AB SALTS 20X (12E) | mL | | | |
| Benomyl | mg | | | |
| pH | | | | |

TABLE 12

| | Units | | | | |
|---|---|---|---|---|---|
| | per | | | | |
| Medium Components | liter | 13266K | 272X | 272V | 13158 |
| MS BASAL SALT MIXTURE | g | 4.3 | 4.3 | 4.3 | 4.3 |
| N6 MACRONUTRIENTS 10X | mL | 60.0 | | | |
| POTASSIUM NITRATE | g | 1.7 | | | |
| B5H MINOR SALTS 1000X | mL | 0.6 | | | |
| NaFe EDTA FOR B5H 100X | mL | 6.0 | | | |
| ERIKSSON'S VITAMINS | mL | 0.4 | | | |
| 1000X | | | | | |
| S&H VITAMIN STOCK 100X | mL | 6.0 | | | |
| THIAMINE•HCL | mg | 0.5 | | | |
| L-PROLINE | g | 2.0 | | | |
| CASEIN HYDROLYSATE | g | 0.3 | | | |
| (ACID) | | | | | |
| SUCROSE | g | 20.0 | 40.0 | 40.0 | 40.0 |
| GLUCOSE | g | 0.6 | | | |
| MALTOSE | g | | | | |
| 2,4-D | mg | 1.6 | | | |
| AGAR | g | 6.0 | 6.0 | 6.0 | 6.0 |
| PHYTAGEL | g | | | | |
| DICAMBA | g | 1.2 | | | |
| SILVER NITRATE | mg | 1.7 | | | |
| AGRIBIO Carbenicillin | mg | 2.0 | | | |
| Timentin | mg | | | | |
| Cefotaxime | mg | 25 | | | |
| MYO-INOSITOL | g | | 0.1 | 0.1 | 0.1 |
| NICOTINIC ACID | mg | | | | |
| PYRIDOXINE•HCL | mg | | | | |
| VITAMIN ASSAY | g | | | | |
| CASAMINO ACIDS | | | | | |
| MES BUFFER | g | | | | |
| ACETOSYRINGONE | μM | | | | |
| ASCORBIC ACID 10 | mg | | | | |
| MG/ML (7S) | | | | | |
| MS VITAMIN STOCK SOL. | mL | | 5.0 | 5.0 | 5.0 |
| ZEATIN | mg | | | | |
| CUPRIC SULFATE | mg | | | | |
| IAA 0.5 MG/ML (28A) | mL | | | | |
| ABA 0.1 mm | mL | | | | |
| THIDIAZURON | mg | | | | |
| AGRIBIO Carbenicillin | mg | | | | |
| PPT(GLUFOSINATE-NH4) | mg | | | | |
| BAP | mg | | | | |
| YEAST EXTRACT (BD Difco) | g | | | | |
| PEPTONE | g | | | | |
| SODIUM CHLORIDE | g | | | | |
| SPECTINOMYCIN | mg | | | | |
| FERROUS SULFATE•7H20 | mL | | | | |
| AB BUFFER 20X (12D) | mL | | | | |
| AB SALTS 20X (12E) | mL | | | | |
| Benomyl | mg | | | | 100.0 |
| pH | | | 0.5 | 5.6 | |

TABLE 13

| | Units | | | | |
|---|---|---|---|---|---|
| | per | | | | |
| Medium Components | liter | 12R | 810K | 700J | 710N |
| 404 Modified MS Basal Salts | | | | | |
| MS BASAL SALT MIXTURE | g | | | | 4.3 |
| N6 BASAL SALTS | g | | | | |
| N6 MACRONUTRIENTS 10X | mL | | | | |
| POTASSIUM NITRATE | g | | | | |
| B5H MINOR SALTS 1000X | mL | | | | |
| NaFe EDTA FOR B5H 100X | mL | | | | |
| ERIKSSON'S VITAMINS | mL | | | | |
| 1000X | | | | | |
| S&H VITAMIN STOCK | g | | | | |
| THIAMINE•HCL | mg | | | | 1.0 |
| L-PROLINE | g | | | | 0.7 |
| CASEIN HYDROLYSATE | g | | | | |
| (ACID) | | | | | |
| SUCROSE | g | | | | |
| GLUCOSE | g | 5.0 | | | 10.0 |
| MALTOSE | g | | | 5.0 | 20.0 |
| 2,4-D | mg | | | | 2.0 |
| AGAR | g | 15.0 | | | 8.0 |
| BACTO-AGAR | g | | 15.0 | | |
| PHYTAGEL | g | | | | |
| DICAMBA | g | | | | |
| SILVER NITRATE | mg | | | | |
| AGRIBIO Carbenicillin | mg | | | | |
| Timentin | mg | | | | |
| Cefotaxime | mg | | | | |
| MYO-INOSITOL | g | | | | 0.1 |
| NICOTINIC ACID | mg | | | | 0.5 |
| PYRIDOXINE•HCL | mg | | | | 0.5 |
| VITAMIN ASSAY CASAMINO | g | | | | |
| ACIDS | | | | | |
| MES BUFFER | g | | | | 0.5 |
| ACETOSYRINGONE | μM | | | | 100.0 |
| ASCORBIC ACID 10 | mg | | | | 10.0 |
| MG/ML (7S) | | | | | |
| MS VITAMIN STOCK SOL. | mL | | | | |
| ZEATIN | mg | | | | |
| CUPRIC SULFATE | μM | | | | 100.0 |
| IAA 0.5 MG/ML (28A) | mL | | | | |
| ABA 0.1 mm | mL | | | | |
| 12N-a-NAA 1 MG/ML | mg | | | | |
| THIDIAZURON | mg | | | | |
| PPT(GLUFOSINATE-NH4) | mg | | | | |
| BAP | mg | | | | 1.0 |
| YEAST EXTRACT (BD Difco) | g | | 5.0 | | |
| PEPTONE | g | | 10.0 | | |
| SODIUM CHLORIDE | g | | 5.0 | | |
| SPECTINOMYCIN | mg | 50.0 | 50.0 | | |
| FERROUS SULFATE•7H20 | mL | 2.0 | | | |
| AB BUFFER 20X (12D) | mL | 50.0 | | | |
| AB SALTS 20X (12E) | mL | 50.0 | | | |
| THYMIDINE | mg | 50.0 | 50.0 | 50.0 | 50.0 |
| GENTAMYCIN | mg | 50.0 | 50.0 | | |
| Benomyl | mg | | | | |
| Magnesium Sulfate, Anhydrous | g | | | 1.204 | |
| 17F-MEROPENEM | mg | | | | |
| 7V-Vitamin E in EtOH 1 mg/mL | mg | | | | |
| 28E-IBA 1 MG/ML | mg | | | | |
| pH | | | 6.8 | | 5.8 |

TABLE 14

| | Units | | | | |
|---|---|---|---|---|---|
| | per | | | | |
| Medium Components | liter | 605B | 13329B | 404J | 900 |
| 404 Modified MS Basal Salts | | | | 4.96 | |
| MS BASAL SALT MIXTURE | g | 4.3 | 4.3 | | 2.165 |
| N6 BASAL SALTS | g | | | | |
| N6 MACRONUTRIENTS 10X | mL | 60.0 | | | |
| POTASSIUM NITRATE | g | 1.7 | | | |
| B5H MINOR SALTS 1000X | mL | 0.6 | | | |
| NaFe EDTA FOR B5H 100X | mL | 6.0 | | | |
| ERIKSSON'S VITAMINS | mL | 0.4 | | | |
| 1000X | | | | | |
| S&H VITAMIN STOCK | g | 0.6 | | | |
| THIAMINE•HCL | mg | 0.2 | | 0.1 | |
| L-PROLINE | g | 2.0 | 0.7 | | |
| CASEIN HYDROLYSATE | g | 0.3 | | | |
| (ACID) | | | | | |
| SUCROSE | g | 20.0 | 60.0 | 65.0 | 20 |
| GLUCOSE | g | 0.6 | | | |
| MALTOSE | g | | | | |
| 2,4-D | mg | 0.8 | | | |
| AGAR | g | 6.0 | 8.0 | 6.0 | 5 |
| BACTO-AGAR | g | | | | |
| PHYTAGEL | g | | | | |
| DICAMBA | g | 1.2 | | | |
| SILVER NITRATE | mg | 3.4 | | | |
| AGRIBIO Carbenicillin | mg | | | | |
| Timentin | mg | | | | |
| Cefotaxime | mg | | | | |
| MYO-INOSITOL | g | | 0.1 | 0.1 | |
| NICOTINIC ACID | mg | | | | |
| PYRIDOXINE•HCL | mg | | | | |
| VITAMIN ASSAY | g | | | | |
| CASAMINO ACIDS | | | | | |
| MES BUFFER | g | | | | |
| ACETOSYRINGONE | μM | | | | |
| ASCORBIC ACID 10 | mg | | | | |
| MG/ML (7S) | | | | | |
| MS VITAMIN STOCK SOL. | mL | | 5 | 5 | |
| ZEATIN | mg | | 0.5 | | |
| CUPRIC SULFATE | μM | | 4.0 | | |
| IAA 0.5 MG/ML (28A) | mL | | 1 | | |
| ABA 0.1 mm | mL | | | | |
| 12N-a-NAA 1 MG/ML | mg | | | 0.5 | |
| THIDIAZURON | mg | | | | |
| PPT(GLUFOSINATE-NH4) | mg | | | | |
| BAP | mg | | 1.0 | | |
| YEAST EXTRACT (BD Difco) | g | | | | |
| PEPTONE | g | | | | |
| SODIUM CHLORIDE | g | | | | |
| SPECTINOMYCIN | mg | | | | |
| FERROUS SULFATE•7H20 | mL | | | | |
| AB BUFFER 20X (12D) | mL | | | | |
| AB SALTS 20X (12E) | mL | | | | |
| THYMIDINE | mg | | | | |
| GENTAMYCIN | mg | | | | |
| Benomyl | mg | | | | 50 |
| Magnesium Sulfate, Anhydrous | g | | | | |
| 17F-MEROPENEM | mg | 10.0 | 10.0 | 10.0 | 10 |
| 7V-Vitamin E in EtOH 1 mg/mL | mg | | | 1.0 | |
| 28E-IBA 1 MG/ML | mg | | | 1.0 | |
| pH | | 5.8 | | 5.8 | |

### Example 3: Particle Bombardment

Standard protocols for particle bombardment (Finer and McMullen, 1991, In Vitro Cell Dev. Biol.—Plant 27:175-182) can be used with the methods of the disclosure.

### A. Particle-Mediated Delivery for Cas9-Mediated Donor Template Integration Via Homology-Dependent Repair (HDR)

Four plasmids were typically used for each particle bombardment; 1) the donor plasmid (50 ng/μl) containing the donor cassette flanked by homology-arms (genomic sequence) for CRISPR/Cas9-mediated homology-dependent SDN3, 2) a plasmid (50 ng/μl) containing the expression cassette UBI PRO::Cas9::pinII plus an expression cassette ZM-U6 PRO::gRNA::U6 TERM, 3) a plasmid (10 ng/μl) containing the expression cassette 3×ENH::UBI PRO::ODP2, and 4) a plasmid (5 ng/μl) containing the expression cassette NOS::WUS2::IN2 TERM. To attach the DNA to 0.6 μm gold particles, the four plasmids were mixed by adding 10 μl of each plasmid together in a low-binding microfuge tube (Sorenson Bioscience 39640T) for a total of 40 μl. To this suspension, 50 μl of 0.6 μm gold particles (30 μg/μl) and 1.0 μl of Transit 20/20 (Cat No MIR5404, Mirus Bio LLC) were added, and the suspension was placed on a rotary shaker for 10 minutes. The suspension was centrifuged at 10,000 RPM (˜9400×g) and the supernatant was discarded. The gold particles were re-suspended in 120 μl of 100% ethanol, briefly sonicated at low power and 10 μl was pipetted onto each carrier disc. The carrier discs were then air-dried to evaporate away all the remaining ethanol. Particle bombardment was performed using a PDF-1000/HE Particle Delivery Device, at 27 inches Hg using a 600 PSI rupture disc.

A transgenic Pioneer Stiff-Stalk inbred PHH5E was used in this experiment. Hemizygous seed was selected based on seed-specific expression of AM-CYAN1 and was surface sterilized using 80% ethanol for 3 minutes, followed by incubation in a solution of 50% bleach+0.1% Tween-20 while agitating with a stir-bar for 20 minutes. The sterile seed were then rinsed 3 times in sterile double-distilled water. Surface-sterilized seed were germinated on 13158F solid medium under (120 μE m-2 s-1) lights using an 18-hour photoperiod at 25° C.

Alternatively, chlorine gas or oxidizing agents can be used for seed sterilization. Chlorine gas can be generated using a variety of compounds (or agents), including bleaching powders, calcium hypochlorite, sodium hypochlorite, industrial bleach, household bleach, chlorine dioxide monochloramine, dichloramine, and trichloramine. Oxidizing agents that can be used in the method include but are not limited to, ozone, hydrogen peroxide, hypochlorous acid, hypobromous acid, chlorine dioxide, and ethylene dioxide.

After 14 days, the 3 cm segment directly above the seedling mesocotyl was excised (containing the leaf-whorl tissue directly above the apical meristem region of the stem). The 3 cm segment was bisected longitudinally using a scalpel. Then the outer layer of leaf tissue (coleoptile) was discarded. For the leaf tissue/segments derived from each seedling, the leaves were separated and laid flat within a 2 cm diameter in the middle of a culture plate containing one of the two following media; i) medium 13224 containing 12% sucrose for 3-4 hours before bombardment (10 plates, each containing segments/tissue from one of 10 seedlings and, ii) medium 13224C containing 12% sucrose+0.1 mg/l ethametsulfuron for 2-3 hours before bombardment (10 plates, each containing tissue/segments from one of 10 seedlings).

Preparation of DNA-functionalized gold particles was done as follows. Stock solutions of plasmids PHP71193 (SEQ ID NO:240) and PHP71788 (SEQ ID NO:241) (100 ng/μl) were diluted to 50 ng/μl with sterile water. Stock solutions of PHP21875 (SEQ ID NO:242) and PHP40828 (SEQ ID NO:341) (100 ng/μl) were diluted to 25 ng/μl with sterile water. Using sterile, low-binding Eppendorf tubes. Ten μl each of the diluted plasmids PHP71788 (50 ng/μl), PHP71193 (50 ng/μl), PHP21875 (25 ng/μl), and PHP40828 (25 ng/μl), were added to a sterile, low-binding Eppendorf tube (final ratio of plasmids was 50:50:25:25, respectively). This DNA mixture was then added to a sterile-low-binding Eppendorf tube containing 50 μl of 0.6 μM gold particles at a stock concentration of 10 mg/ml) and gently agitated to mix the DNA and gold in the suspension. One μl of Transit 20/20 was added and the tube again gently agitating. The tube was then placed on a 125 RPM rotator shaker for 10 minutes at room temperature. The tube was then centrifuged at 10,000 RPM in a microfuge. The supernatant was discarded and after adding 120 μl of 95% EtOH, the tube was sonicated briefly on a low setting to resuspend the particles and then 10 μl of the DNA/gold/EtOH suspension was pipetted onto the center of the carrier disc. The carrier discs were left exposed to the sterile air low in the laminar flow hood for approximately 10 minutes to evaporate the EtOH. The carrier discs with dried gold/DNA were then used for particle bombardment. For particle bombardment, a PDS-1000/He Particle Delivery System (Bio-Rad, Hercules, CA) was used, with 425 psi rupture disc, and the petri dish containing the target tissue/segments positioned two shelves below the carrier-holder, and a vacuum of approximately 27 mg Hg.

When expression of Wus2 and Odp2 was induced by addition of ethametsulfuron, somatic embryogenesis was stimulated in leaf segments/tissue. Using this inducible Wus2/Odp2 germplasm as the starting point for a new experiment, seedling-derived leaf segments/tissue was then used as the target explant for particle bombardment. As mentioned above, in one treatment the leaf segments/tissue was incubated on culture medium with 12% sucrose (to plasmolyze the leaf cells) prior to particle bombardment, and in the second treatment the leaf pieces/segments were exposed to culture medium with 12% sucrose plus 0.1 mg/l ethametsulfuron prior to particle delivery (providing an earlier exposure to the inductive treatment to begin stimulation of Wus2/Odp2 expression). To further enhance morphogenesis (beyond that provided by inducible expression), plasmids containing constitutive Wus2 and ODP2 expression cassettes were co-delivered with Cas9 and gRNA, as well as the template DNA (the genomic-sequence-flanked NPTII expression cassette). After DNA delivery, successful NPTII coding sequence integration via homology-dependent recombination (HDR) permitted regeneration of HDR events using both the inducing ligand (0.1 mg/l ethametsulfuron) and G418 for selection. Due to high levels of Wus2 and Bbm expression (inducible-expression from pre-integrated 60850-T-DNA plus constitutive provided by PHP21875 and PHP40828), selection using NPTII and G418 became less efficient, resulting in escape (wild type) plants being recovered. Thus, at lower levels of G418 selective agent (150 or 200 mg/l), when leaf tissue/segments from 9 seedlings was used as starting explants for each treatment, 46 and 34 T0 plants containing the NPTII gene were recovered but none were observed to contain perfect HDR integrations. In contrast, when 9 seedlings were again used for particle delivery of the plasmids followed by increased selective pressure due to higher G418 (250 mg/l), selection became more stringent and three perfect HDR integration events were recovered from a total of 38 T0 plants that were regenerated and analyzed.

Thus, using this combination of Wus2 and Odp2 expression cassettes to stimulate growth while also delivering the SDN3 donor DNA, the Cas9 expression cassette, and the guide-RNA expression cassette resulted in efficient homology-dependent targeted integration. Thus, three perfect HDR events were recovered from particle bombardment of leaf segments derived from only 34 starting seedlings.

In comparison, when wild-type maize Stiff-Stalk inbred PHH5G was transformed in a similar manner but without the use of Wus2 and Odp2, transgenic events were not recovered. Thus, particle delivery of the plasmids PHP71193 and PHP71788 into seedling-derived leaf segments/tissue (with no Wus2 or Odp2) does not result in transgenic or edited T0 plants.

### B. Site-Specific Integration

Pioneer inbred PH184C (disclosed in U.S. Pat. No. 8,445,763, incorporated herein by reference in its entirety) that contains in chromosome-1 a pre-integrated Site-Specific Integration (SSI) target site (Chrom-1 target site) composed of UBI PRO:FRT1:NPTII::PINII TERM+FRT87 is used. Prior to bombardment, 10-12 DAP (days after pollination) immature embryos are isolated from ears of Pioneer inbred PH184C and placed on 605J culture medium plus 16% sucrose for three hours to plasmolyze the scutellar cells. Alternatively, the first 2-3 cm of seedling-derived leaf-whorl tissue is bisected longitudinally and sliced into approximately 0.5-3.0 mm leaf segments, and these leaf segments are plasmolyzed on 605J medium plus 16% sucrose for three hours prior to particle bombardment.

Four plasmids are typically used for each particle bombardment:
1) a donor plasmid (100 ng/μl) containing a FRT-flanked donor cassette for Recombinase-Mediated Cassette Exchange, for example a plasmid containing FRT1:PMI::PINII TERM::CZ19B1 TERM+UBI1ZM PRO::UBI1ZM 5 UTR::UBI1ZM INTRON1::DS-RED2::PINII TERM+FRT6 (PHP8418-0004; SEQ ID NO:92);
2) a plasmid (2.5 ng/μl) containing the expression cassette UBI1ZM PRO::UBI1ZM 5 UTR::UBI1ZM INTRON1::FLPm::PINII TERM (PHP5096; SEQ ID NO:3);

3) a plasmid (10 ng/μl) containing the expression cassette ZM-PLTP PRO::ZM-ODP2::OS-T28 TERM+FMV & PCSV ENHANCERS (PHP89030; SEQ ID NO:12); and
4) a plasmid (5 ng/μl) containing the expression cassette ZM-PLTP PRO::ZM-WUS2::IN2-1 TERM+PSW1+GZ-W64A TERM+FL2 TERM (PHP89179; SEQ ID NO:13).

To attach the DNA to 0.6 μm gold particles, the four plasmids are mixed by adding 10 μl of each plasmid together in a low-binding microfuge tube (Sorenson Bioscience 39640T) for a total of 40 μl. To this suspension, 50 μl of 0.6 μm gold particles (30 μg/μl) and 1.0 μl of Transit 20/20 (Cat No MIR5404, Mirus Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all remaining ethanol. Particle bombardment is performed using a Biolistics PDF-1000, at 28 inches of Mercury using a 200 PSI rupture disc. After particle bombardment, the immature embryos or leaf segments are selected on 605J medium modified to contain 12.5 g/l mannose and 5 g/l maltose and no sucrose. After 10-12 weeks on selection, plantlets are regenerated and analyzed using qPCR. Co-delivery of PLTP::ODP2 (PHP89030; SEQ ID NO: 12) and PLTP::WUS2 (PHP89179; SEQ ID NO:13) along with the SSI components (Donor DNA (PHP8418-0004; SEQ ID NO:92)+UBI::FLP (PHP5096; SEQ ID NO:3) is observed to produce high frequencies of site-specific integration of the donor fragment into the Chrom-1 target site (i.e. at rates of 4-7% relative to the number of bombarded immature embryos).

Particle bombardment methodologies described herein, in combination with any pre-treatment of seedlings described in the present application is observed to produce high transformation frequencies due to leaf segments that exhibit an improvement in frequencies of T-DNA delivery, somatic embryo response (more rapid growth and higher numbers), production of T0 plants, and single-copy integration frequencies.

### Example 4: Agrobacterium-Mediated Transformation of Corn

### A. Preparation of Agrobacterium Master Plate.

Agrobacterium tumefaciens harboring a binary donor vector was streaked out from a −80° C. frozen aliquot onto solid 12R medium and cultured at 28° C. in the dark for 2-3 days to make a master plate.

### B. Growing Agrobacterium on Solid Medium.

A single colony or multiple colonies of Agrobacterium were picked from the master plate and streaked onto a second plate containing 810K medium and incubated at 28° C. in the dark overnight.

Agrobacterium infection medium (700A; 5 ml) and 100 mM 3′-5′-Dimethoxy-4′-hydroxyacetophenone (acetosyringone; 5 μL) were added to a 14 mL conical tube in a hood. About 3 full loops of Agrobacterium from the second plate were suspended in the tube and the tube was then vortexed to make an even suspension. The suspension (1 ml) was transferred to a spectrophotometer tube and the optical density (550 nm) of the suspension was adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were then used as soon as possible.

### C. Growing Agrobacterium on Liquid Medium.

Alternatively, Agrobacterium can be prepared for transformation by growing in liquid medium. One day before infection, a 125 ml flask was prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate was suspended into the flasks and placed on an orbital shaker set at 200 rpm and incubated at 28° C. overnight. The Agrobacterium culture was centrifuged at 5000 rpm for 10 min. The supernatant was removed and the Agrobacterium infection medium (700A) with acetosyringone solution was added. The bacteria were resuspended by vortex and the optical density (550 nm) of the Agrobacterium suspension was adjusted to a reading of about 0.35 to 2.0.

### D. Maize Transformation.

Maize seed was surface-sterilized for 15-20 min in 20% (v/v) bleach (5.25% sodium hypochlorite) plus 1 drop of Tween 20 followed by 3 washes in sterile water, germinated and allowed to grow into seedlings for approximately 14 days, and then prepared to produce leaf segments/fragments as described above. Leaf segments were placed in the Agrobacterium infection medium (700A) with 200 μM acetosyringone solution+0.02% surfactant solution (Break-Thru® surfactant, Plant Health Technologies, Boise, ID). The Agrobacterium infection medium was drawn off and 1 ml of the Agrobacterium suspension was added to the leaf segments and was allowed to stand for 20 min. The suspension of Agrobacterium and leaf segments were poured through a sterile metal sieve and the liquid was discarded. The leaf segments/pieces collected on the metal sieve were transferred using a spatula onto a stack of 3 sterile Whatman #2 filter papers, used to wick off excess Agrobacterium-containing liquid, and then again, a spatula was used to transfer the leaf pieces/segments onto a filter paper lying on co-cultivation medium. The plate was incubated in the dark at 21° C. for 1-3 days of co-cultivation.

The filter papers supporting the leaf segments were then transferred to resting medium (605T medium) without selection. Seven days later, the filter papers supporting the leaf segments were transferred to selection medium for three weeks. After selection, healthy growing somatic embryos were transferred using forceps onto maturation medium for two weeks in the dark, at which point the maturation plates were transferred in to (still containing the maturing somatic embryos) into the light for an addition week. After one week in the light, regenerating plantlets were transferred to rooting medium. After rooting, plantlets were ready for transplanting to the greenhouse.

### Example 5: Transformation of Maize Leaf Segments

Constitutive expression of WUS2 and ODP2 after Agrobacterium-mediated transformation of maize leaf segments resulted in production of embryogenic callus and/or rapidly formed somatic embryos which regenerate into healthy, fertile T0 plants.

The general protocol for Agrobacterium-mediated maize transformation described in Example 4 was used, with the modifications described below for using leaf tissue/segments as the target explant.

### A. In Vitro Seed Germination to Produce Seedling Target Tissue

Mature seeds were surface sterilized by immersion in a series of solutions under agitation using a magnetic stir bar; first in an 80% ethanol solution for 3 minutes, the ethanol solution was decanted and replaced with a 30% Clorox bleach solution containing 0.1% Tween-20 for 20 minutes, the Clorox bleach solution was decanted, and the mature seeds were rinsed (three 5-minute rinses) in autoclaved sterile water. The sterilized seeds were transferred onto solid 900 medium after the final sterile water rinse. In vitro germination and seedling growth were carried out at 26° C. with a 16 h light/8 h dark photoperiod. The first 2.5 to 3 cm of leaf whorl above the mesocotyl was removed from each 12-18 day-old seedling for further processing for transformation.

### B. Agrobacterium Preparation

Agrobacterium tumefaciens strain LBA4404 TD THY-harboring PHP71539 (SEQ ID NO: 4; pVIR9, see US20190078106A1, herein incorporated by reference in its entirety) and a binary donor vector, PHP96037 (SEQ ID NO:66), containing a WUS2/ODP2 T-DNA with a selectable marker (ZM-ALS (HRA)) and a screenable marker (ZS-GREEN1) or a binary donor control vector containing a selectable marker and/or a screenable marker T-DNA (lacking WUS2/ODP2) was streaked out from a −80° C. frozen aliquot onto solid 12V medium and cultured at 28° C. in the dark for 2 days to make a master plate. A working plate was prepared by streaking 4-5 colonies from the 12V-grown master plate across fresh 810K media, incubating overnight in the dark at 28° C. prior to using for Agrobacterium infection.

Agrobacterium infection medium (700J medium, 10 ml) with the addition of 20 μL of acetosyringone and 20 μL of a previously 10-fold-diluted surfactant solution (Break Thru S 233, Evonik Industries GmbH, Goldschmidtstraße 100, 45127 Essen, Germany) was added to a 50 mL conical tube in a hood. About 5 full loops of Agrobacterium were collected from the working plate, transferred to the infection medium in the 50 ml tube, and then vortexed until uniformly suspended. The suspension (1 ml) was transferred to a spectrophotometer tube and the optical density (550 nm) of the suspension was adjusted to a reading of 0.6. The final Agrobacterium suspension was aliquoted into Corning eight-well plates containing 0.4 μm permeable culture inserts (Falcon, Part Numbers 353046 and 353090, respectively) with each well containing about 8 mL of the Agrobacterium suspension.

### C. Manual Preparation of Leaf Segments/Pieces and Transformation

Seed of maize inbred PH85E were surface sterilized as previously described, and then germinated at 28° C. under low light on solid 90B medium (½ strength MS salts plus 20 g/l sucrose and 50 mg/l benomyl). The leaf base segment (an approximate 2.5-3.0 cm section above the mesocotyl) was removed from each 12-18 day-old in vitro-germinated seedling with sterilized scissors. These leaf segments were placed into a 150 mm×15 mm Petri dish. Forceps were used to hold each leaf whorl section at the upper green end and the section was bisected longitudinally into 2 lengthwise halves using a sterile #10 scalpel blade. The outer leaf was removed and the inner leaves of the whorl were then cross-cut (diced) into smaller sections (approximately 1 to 3 mm in size, preferably 2.5-3.0 mm in size). Small leaf sections were collected and directly transferred into the permeable culture inserts containing the Agrobacterium suspension and incubated at room temperature (25° C.) for a 15-minute infection period.

After infection, the culture insert containing the Agrobacterium-infected leaf segments was removed from the 8-well plate and placed on an autoclaved dry filter paper to wick up and remove any residual Agrobacterium solution. The infected leaf segments were then transferred onto a fresh filter paper (VWR 7.5 CM) resting on 710N solid co-cultivation medium. Forceps were used to evenly disperse the leaf segments on the 710N plates and to ensure they have enough room to grow. The infected leaf segments/tissue was incubated at 21° C. in the dark for 2-3 days. After 2-3d co-cultivation, the paper supporting the leaf segments/tissue was removed from the 710N medium and transferred onto 605B medium for 4 week resting culture. Leaf segments/tissue was sub-cultured every 2 weeks. After the 4 weeks culture on resting medium (605B) the plates were placed into a controlled temperature/humidity incubator (45° C./70% RH) for a 2-hour heat treatment. The plates were removed from the incubator and kept at room temperature (25° C.) for 1-2 hours until the plates had cooled down. Depending on the maize inbred, a single two-hour heat treatment, or two 2-hour heat treatments on two consecutive days, were applied to stimulate the drought-inducible RAB17 promoter and induce CRE-mediated excision of WUS2, ODP2, and CRE recombinase.

### D. Mechanized Production of Leaf Segments/Pieces

As an alternative to manual slicing of longitudinally-bisected leaf whorl segments from seedlings, the final 0.5-3. mm leaf pieces/segments were mechanically produced as follows. First, the seedlings were cut above the mesocotyl (removing the aerial portions from the roots) and the first 3 cm of leaf whorl was harvested, discarding the remainder of the more mature leaf tissue. The 3-cm long leaf whorl was bisected longitudinally using a scalpel, and the halves were put into 100 ml of Agrobacterium suspension (OD=0.5-0.6 measured at 550 nm, with the bacterium suspended in medium 700J+100 mM AS+0.02% surfactant solution (Break-Thru® surfactant) in a food processor. The leaf tissue was pulse-blended on low speed (10 pulses) until the average size of leaf fragments/segments were approximately 0.5-3 mm in length/depth. The suspended leaf tissue/segments in the Agrobacterium suspension remained in the blender bowl for 20 minutes at room temperature with gentle swirling every 1-2 minutes, which constituted the “Agrobacterium Infection” step. After infection, the suspension was poured through a sterile stainless-steel screen, catching the leaf segments/fragments from the liquid that passed through for disposal. The leaf segments were then transferred from the screen onto three layers of dry Whatman's #2 filter papers which wicked away excess Agrobacterium suspension (but not being washed) so that a thin layer of bacterium remained on the surface of the leaf segments/pieces. The leaf segments/pieces were again transferred onto a single layer of Whatman's filter paper resting on solid co-cultivation medium (710N) and were then cultured in the dark at 210C for 24 hours. After co-cultivation, the filter papers with the supported leaf segments/pieces were transferred onto resting medium 605B and cultured in the dark at 28° C. for one week, at which point the filter papers were again transferred onto selection medium 13266N and cultured in the dark at 28° C. for 3 weeks. After selection, the selection plated (held in a translucent culture box, typically holding 12 plates in 6 stacks of 2 plates) was transferred into a 45° C., 70% relative humidity incubator for two hours, then removed and the box placed on a benchtop at 25° C. for 1.5 hours for the temperature to re-equilibrate to room temperature. After heat treatment (which activated HSP17.7 PRO::CRE expression for excision of WUS/BBM/CRE from the T-DNA) healthy somatic embryos were transferred from the subtending filter papers onto fresh maturation medium 13329B and cultured for 2 weeks at 28° C. in the dark, then the plates were transferred into the light (120 μE m-2 s-1, 18-hour photoperiod) at 25° C. for one additional week. Healthy mature somatic embryos that had begun producing shoots were then transferred onto rooting medium 404J for an additional 203 weeks of culture under lights. Plantlets were then transferred to soil in the greenhouse. When regenerated T0 plants were large enough for sampling, leaf tissue was punched for qPCR analysis for T-DNA and Agrobacterium plasmid backbone sequences.

After the heat treatment and temperature equilibration at room temperature, leaf segments with newly developed somatic embryos were transferred onto 13329B maturation medium without filter papers, cultured in the dark at 28° C. for 2 weeks, and then moved into a 26° C. light room for an additional week. Leaf segments that now supported small shoots were transferred onto 404J rooting medium for an additional 2-3 weeks until well formed roots had developed, at which point the plantlets were ready for transfer to the greenhouse.

Transformation efficiency (transformation frequency) was calculated as the number of independent transgenic T0 plants produced per number of starting seedlings used for leaf fragment/segment preparation on a percentage basis. For example, 50 seedlings were used and separated into 5 groups (for five different treatments in an experiment) of 10 seedlings/treatment (or experimental replicates as shown in Table 15). For each seedling within a group, a 3 cm cylinder of wrapped leaf tissue above the mesocotyl was excised and each cylinder was bisected longitudinally. These lengths of bisected leaf tissue were then manually sliced with a scalpel or placed into liquid within a food processor and pulsed, both methods produced leaf fragments/segments of between 0.5-3.0 mm in length on average. The number of final leaf segments (fragments) used for transformation per starting seedling could be variable depending on the size and breadth of the seedling leaves, the physical cutting process which varied slightly from batch to batch, etc. It should also be noted that based on this procedure the leaf segments/fragments from each cohort of 10 seedlings within each treatment (or replicate) were pooled for Agrobacterium-mediated transformation.

An independent transgenic T0 event identified by positive PCR analysis was tabulated as a molecularly unique T0 plant produced from a single leaf segment/fragment, which precluded counting clonal events (the same transgenic integration pattern for example) as separate events. Once the final number of molecularly characterized transgenic events for a given treatment had been determined, the final number of transgenic T0 plants (independent events) were totaled and divided by the number of starting seedlings for that replicate (10 in this Example 5) and the product was multiplied by 100 to provide a percentage. Thus, for experimental replicate 5 in Table 15, 30 T0 plants were produced from 10 starting seedlings, for a transformation frequency (T0% in Table 15) of (30/10)×100=300%.

Results from five experiments are shown in Table 15, in which 10 starting seedlings per experiment (50 total) were used to produce the starting leaf segments for Agrobacterium infection, the number of transgenic T0 plants recovered ranged from 18 (Exp. 1) to 51 (Exp. 4), resulting in a mean transformation frequency of 360%+/−112 Standard Deviation (SD). This is in contrast to experiments in which only a selectable marker gene and/or a screenable marker gene (fluorescent protein gene) were contained in the T-DNA, in which no culture response was observed and no T0 plants were produced.

In addition to a high transformation frequency, a high percentage of the recovered TO plants were single-copy (SC) for the T-DNA (containing the selectable marker and/or the screenable marker) with no contaminating sequences from Agrobacterium being detected. Such SC/No-Agro events (T0 plants) ranged from 23% to 37% with a mean of 31.4% (+/−5.2% SD). By comparing the number of high-quality transgenic T0 plants (SC for the T-DNA with no contaminating Agrobacterium backbone sequences) to the number of starting seedlings used in these experiments provided a clear measure of overall efficiency, with a mean frequency of 114% (+/−44% SD). This method using WUS2/ODP2 obviated the need for growing mature maize plants for 90-120 days in the greenhouse to produce immature embryo explants for transformation and provided transgenic events from leaf explants generated from germinated seed in the lab.

TABLE 15

| | | # T0 | | | | SC No- | |
|---|---|---|---|---|---|---|---|
| | # | plants | | # SC No- | SC No- | Agro | Escape |
| Exp | Seedlings | (events) | T0% | Agro Seq | Agro %* | %** | Freq. |
| 1 | 10 | 18 | 180% | 5 | 28% | 50% | 39% |
| 2 | 10 | 41 | 410% | 14 | 34% | 140% | 32% |
| 3 | 10 | 40 | 400% | 9 | 23% | 90% | 45% |
| 4 | 10 | 51 | 510% | 18 | 35% | 180% | 40% |
| 5 | 10 | 30 | 300% | 11 | 37% | 110% | 43% |
| Total | 50 | 180 | 360% | 57 | 29% | 114% | 39% |

*Frequency of T0 plants with single-copy T-DNA and no plasmid sequences, relative to the total number of T0 plants
**Frequency of T0 plants with single-copy T-DNA and no plasmid sequences, relative to the starting number of seedlings

### Example 6: Transformation of Sorghum Leaf Segments

Constitutive expression of WUS2 and ODP2 after Agrobacterium-mediated transformation of sorghum leaf segments results in production of embryogenic callus and/or rapidly formed somatic embryos which regenerate into healthy, fertile T0 plants.

Agrobacterium strain, constructs, growth of seedlings, preparation of leaf material for transformation, Agrobacterium infection, co-culture, resting culture, maturation and rooting for sorghum were all the same as the methods developed for maize in EXAMPLE 5. The purpose here was to determine how transferable the method was without any sorghum-specific optimization.

Results from four experiments using a WUS2/ODP2 T-DNA, along with one experiment in which the control T-DNA contained only a selectable marker and a fluorescent marker (HRA+ZS-GREEN) are shown in Table 16. Each experiment also contained a comparison between two resting media, 13266P (605B medium plus 50 mg/l meropenem) which contained no additional cupric sulfate or BAP and medium 13265L (13266P medium plus 100 μM cupric sulfate and 0.5 mg/l BAP).

As demonstrated for maize, sorghum treatments that contained WUS2 and ODP2 expression cassettes in the T-DNA (PHP96037; SEQ ID NO:66) also resulted in high transformation frequencies, calculated based on the number of transgenic T0 plants recovered per starting seedling, with a mean (+/−SD) for 13266P and 13265L of 36.5% (+/−4.1%) and 35.5% (+/−9.6%) respectively, with no significant difference between the two media compositions (p=0.05). In contrast, the control treatment containing the selectable marker and/or the screenable marker with no WUS2/ODP2 in the T-DNA produced no transgenic events. The mean frequency of obtaining high-quality TO sorghum plants (single copy with no Agrobacterium backbone (SC/NA %)) when transformed with PHP96037 (SEQ ID NO:66) was between 36% to 38% for the two media.

As with maize, this method obviated the need for growing mature sorghum plants for 90-120 days in the greenhouse to produce immature embryos explants for transformation and provided transgenic events from leaf explants generated from germinated seed in the lab.

TABLE 16

| | | | | | # | | | |
|---|---|---|---|---|---|---|---|---|
| | | | | | SC/No | | | |
| | # | Treatment | # T0 | | Agro | SC/NA | SC | # |
| Expt. No. | Seedlings | (Medium) | Plants | T0% | (NA) | % | % | Escapes |
| 1 | 15 | 13265L | 65 | 433% | 26 | 40% | 30% | 2 |
| | 13 | 13266P | 18 | 138% | 7 | 39% | 7% | 2 |
| 2 | 15 | 13265L | 56 | 373% | 18 | 32% | 28% | 2 |
| | 15 | 13266P | 46 | 307% | 19 | 41% | 28% | 5 |
| 3 | 15 | 13265L | 32 | 213% | 7 | 22% | 7% | 0 |
| | 15 | 13266P | 14 | 93% | 5 | 36% | 13% | 0 |
| 4 | 16 | 13265L | 27 | 169% | 13 | 48% | 13% | 9 |
| | 16 | 13266P | 20 | 125% | 6 | 30% | 6% | 3 |
| Total | 61 | 13265L | 180 | 295% | 64 | 36% | 78% | 13 |
| Total | 59 | 13266P | 97 | 164% | 37 | 38% | 54% | 10 |
| No | 15 | 13265L | 0 | 0% | 0 | 0% | 0% | 0 |
| WUS/ODP2 | | | | | | | | |
| Control | 15 | 13266P | 0 | 0% | 0 | 0% | 0% | 0 |

### Example 7: Promoter, Additional Helpers, Excision Components, and Selectable Marker Combinations

Using a variety of promoter, additional helpers, excision components, and selectable marker combinations for expression of WUS2 and ODP2 after Agrobacterium-mediated transformation of leaf segments results/resulted in production of embryogenic callus and/or rapidly formed somatic embryos which regenerate/regenerated into healthy, fertile T0 plants.

### A. Constitutive Promoters Combinations

As shown below numerous combinations of promoters, additional helpers, excision components, and selectable markers resulted in successful accelerated leaf transformation in maize.

Maize seedling-derived leaf segments were transformed using Agrobacterium strain LBA4404 TD THY—as described in Example 5. T-DNA delivery was evaluated based on transient expression of UBI-ZS-GREEN, which was present in all of the T-DNA variations tested. Fourteen to twenty-one days after transformation, growth responses were evaluated based on both the rate of growth and the morphology of the leaf segment/tissue (see Table 17 for rating scale). Leaf transformation assay scoring (Assay Score), as shown in Table 17, is based on morphology (early somatic embryo formation versus production of embryogenic callus) and growth rate, with increasing numerical scores indicating more rapid growth, and a concomitant progression from entirely callus growth (i.e., a score of 1) to rapidly producing single functional somatic embryos with no callus (i.e., a score of 4).

TABLE 17

| | Growth | | Percentage of | |
|---|---|---|---|---|
| TXN Resp. | at <21 | | Leaf Segments | |
| Assay Score | days | Morphology | Responding | Description |
| 0 | Single | NA | 0% | Good DNA delivery and transient |
| | Cells | | | expression, but no growth |
| 1 | Slow | Callus | <20% | Slow growing embryogenic callus |
| 2 | Moderate | Callus | 30-50% | Faster growth but still mixed |
| | | | | embryogenic callus |
| 3 | Fast | SE | 60-80% | Some Early Somatic Embryos |
| | | | | (ESE) with rapid growth |
| 4 | Most | SE | 60-80% | Highest density of rapidly growing |
| | Rapid | | | Early Somatic Embryos (ESE) |

Table 18 shows the growth response after Agrobacterium-mediated transformation of maize leaf segments with T-DNAs from plasmids containing different construct combinations of promoters, additional helpers, excision components, and selectable markers.

Of the various constructs tested, 29 constructs resulted in an Assay Score of “2”, while 23 constructs resulted in the rapid production of early somatic embryos within 14-21 days of starting the Agrobacterium infection (an Assay Score of either 3 or 4). These results demonstrated that various promoters, additional helpers, excision components, and selectable marker combinations for WUS2 and ODP2 used in leaf transformations produced a callus growth response and/or a rapid embryo response and resulted in an increased transformation efficiency (percentage of leaf segments responding). However, a subset of 23 plasmids resulted in rapid somatic embryo formation and substantially shortened the duration of the transformation process. This was manifested as a shortened time in culture. Constructs with an Assay Score of two (2) typically produced embryogenic callus that was ready for the maturation phase (where embryo regeneration of shoots begins) within about 6-8 weeks after Agrobacterium infection, while for Assay Scores of three (3) and four (4) this duration was further reduced to 5-7 and 4-6 weeks, respectively. This was compared to the published method of Lowe et al. (2016, Plant Cell 28:1998-2015) for leaf segment transformation where the duration of culture was between 10-12 weeks before somatic embryo maturation was started.

Compared to the Lowe et al. construct, PHP35648 (SEQ ID NO:1), and other constructs tested herein that produced a slow growing callus response requiring 10-12 weeks before somatic embryo maturation, many other constructs tested herein resulted in a shorter time frame to reach the somatic embryo maturation stage (8 weeks or less). Constructs that resulted in a shorter time frame to reach the somatic embryo maturation stage (8 weeks or less) included combinations containing various promoters driving WUS and ODP2 and additional helpers, excision components, and selectable marker combinations as shown in Table 18.

TABLE 18

| | | | | CRE | | |
|---|---|---|---|---|---|---|
| | | | | Position | | TXN |
| | | | | (Up- or | | Resp. |
| | WUS | BBM | Additional | down- | Selectable | Assay |
| Plasmid | Promoter | Promoter | Helper | stream) | Marker | Score |
| PHP46332 | NOS | 35SENH- | | RAB17-- | moPAT | 1 |
| | | UBI | | up | | |
| PHP83652 | AXIG1 | PLTP | | HSP17-- | HRA | 0 |
| | | | | down | | |
| PHP83475 | PLTP | | | HSP17-- | HRA | 0 |
| | | | | down | | |
| PHP83621 | PLTP | PLTP | | GLAB1-- | HRA | 0 |
| | | | | down | | |
| PHP81855 | 8xENH | PLTP: | | RAB17-- | NPTII | 1 |
| | 35S 2xop | 1xOP | | up | | |
| PHP81856 | AXIG1 | PLTP | | RAB17-- | NPTII | 0 |
| | | | | down | | |
| PHP81857 | NOS | PLTP | | RAB17-- | NPTII | 2 |
| | | | | down | | |
| PHP81858 | NOS | UBI | | RAB17-- | NPTII | 2 |
| | | | | down | | |
| PHP92365 | PLTP | | UBI:LEC1 | NO CRE | HRA | 0 |
| PHP92765 | PLTP | | PLTP2:PKL-A | RAB17-- | no | 0 |
| | | | | down | | |
| PHP92928 | 35S- | | UBI:LEC1 | NO CRE | HRA | 0 |
| | ENH:PLTP | | | | | |
| PHP93271 | PLTP | | UBI:REPA | NO CRE | HRA | 0 |
| PHP93559 | NOS | UBI | | RAB17--up | PMI | 1 |
| PHP93613 | ACTIN | | UBI:LEC1 | RAB17--up | HRA | 0 |
| PHP93696 | CAB:2xEME | | excision | HSP17--up | HRA | 0 |
| | | | activate | | | |
| | | | ubi:2xEME:LEC1 | | | |
| PHP93586 | PLTP:WUS-T2A | | PLTP:T2A-REPA | None | HRA | NT |
| pHP93742 | CAB:WUS-T2A | T2A-BBM | CAB:amiPKL-A | None | HRA | 0 |
| PHP93743 | SCBV | | UBI:REP-A | HSP17--up | HRA | 1 |
| PHP93738 | ACTIN | UBI | | HSP17--up | HRA | 2 |
| PHP95385 | ACTIN | UBI | | HSP17-- | HRA | 3 |
| | | | | down | | |
| PHP93766 | NOS | | UBI:LEC1 | HSP17--up | HRA | 0 |
| PHP93925 | UBI | 3xENH- | | NO CRE | HRA | 4 |
| | | UBI | | | | |
| PHP93926 | NOS | UBI | | HSP17--up | HRA | 1 |
| PHP93932 | 3xENH-CAB | | CAB-L:amiPKL-A | NO CRE | HRA | 2 |
| PHP93933 | NOS | 3xENH- | | NO CRE | HRA | 4 |
| | | UBI | | | | |
| PHP93937 | CAB | | CAB-L:amiPKL-A | | HRA | NT |
| PHP93739 | NOS | UBI | | HSP17--up | HRA | 2 |
| PHP94684 | UBI | BI-DIR-UBI | | HSP17--up | HRA | 1 |
| PHP94685 | 3xENH- | BI-DIR-UBI | | HSP17--up | HRA | 1 |
| | UBI | | | | | |
| PHP94638 | UBI | B-DIR- | UBI:amiPKL-A | HSP17--up | NO | 0 |
| | | UBI | | | | |
| PHP94636 | 8xDR5-35S | PLTP:1xop | | RAB17-- | NPTII | 1 |
| | | | | down | | |
| PHP94715 | 8xDR5-35S | UBI | | RAB17-- | NPTII | 2 |
| | | | | down | | |
| PHP94682 | SCBV | | UBI:LEC1 | RAB17-- | HRA | 0 |
| | | | | down | | |
| PHP95067 | NOS | | UBI:CUC1-2 | HSP17-- | HRA | 0 |
| | | | | down | | |
| PHP95068 | NOS | | UBI:CUC2 | HSP17-- | HRA | 0 |
| | | | | down | | |
| PHP95069 | NOS | | UBI:CUC3-2 | HSP17-- | HRA | 0 |
| | | | | down | | |
| PHP95070 | NOS | | UBI:zm-GPCNAC-1 | HSP17-- | HRA | 0 |
| | | | | down | | |
| PHP95071 | NOS | | UBI:RKD4 | HSP17-- | HRA | 0 |
| | | | | down | | |
| PHP95072 | FT-MEM1-NOS | UBI | | HSP17-- | HRA | 3 |
| | | | | down | | |
| PHP95073 | PEPC1 | UBI | | HSP17-- | HRA | 1 |
| | | | | down | | |
| PHP95074 | DIURNA | UBI | | HSP17-- | HRA | 1 |
| | L12 | | | down | | |
| PHP95075 | NOS | | 3xENH-UBI:LEC2 | HSP17-- | HRA | 0 |
| | | | | down | | |
| PHP95205 | RUBISCO | UBI | | HSP17-- | HRA | 1 |
| | SSU | | | down | | |
| PHP94331 | NOS | UBI | CAB:zm-MPKL-A | HSP17--up | HRA | 1 |
| PHP94332 | NOS | UBI | UBI:zm-MPKL-A | HSP17--up | HRA | 1 |
| PHP95393 | CSVMV | UBI | | HSP17-- | HRA | 1 |
| | | | | down | | |
| PHP95394 | CAB | UBI | | HSP17-- | HRA | 1 |
| | | | | down | | |
| PHP95502 | NOS | 3xEME- | | ins-HSP17-- | HRA | 2 |
| | | UBI | | up | | |
| PHP95503 | NOS | 3xEME- | | ins HSP17-- | HRA | 3 |
| | | UBI | | down | | |
| PHP95499 | ACTIN | 3xEME-UBI | | HSP17--up | HRA | 1 |
| PHP96664 | 3xEME-NOS | UBI | | HSP17--up | HRA | NT |
| PHP96695 | NOS | 3xEME- | | HSP17-- | HRA | 4 |
| | | UBI | | down | | |
| PHP97453 | UBI | 3xEME- | | HSP17-- | HRA | 3 |
| | | UBI | | down | | |
| PHP95886 | NOS | UBI | UBI:RKD4 | HSP17-- | HRA | 1 |
| | | | | down | | |
| PHP95892 | NOS | UBI | UBI:RAP2.6L | HSP17-- | HRA | 2 |
| | | | | down | | |
| PHP95881 | NOS | UBI | UBI:LEC2 | HSP17-- | HRA | 0 |
| | | | | down | | |
| PHP95882 | NOS | UBI | UBI:LEC1V1 | HSP17-- | HRA | 1 |
| | | | | down | | |
| PHP95893 | NOS | UBI | UBI:MIR156B | HSP17-- | HRA | 1 |
| | | | | down | | |
| PHP95990 | NOS | | UBIRAP2.6L | HSP17-- | HRA | 1 |
| | | | | down | | |
| PHP95989 | FT- | UBI | | HSP17-- | HRA | 1 |
| | PPCA1- | | | down | | |
| | NOS | | | | | |
| PHP95987 | SCBV | UBI | | HSP17-- | HRA | 1 |
| | | | | down | | |
| PHP96037 | NOS | 3xENH- | AT-5-IV-2 | ins HSP17-- | HRA | 4 |
| | | UBI | (two) | down | | |
| PHP96277 | ACTIN | 3xENH- | AT-5-IV-2 | ins HSP17-- | HRA | 4 |
| | | UBI | (two) | down | | |
| PHP95904 | NOS | UBI | UBI:RLD1 | HSP17--up | HRA | 1 |
| PHP96030 | NOS | | UBI:RLD1 | HSP17--up | HRA | 0 |
| PHP96036 | NOS | | KN1 | HSP17--up | HRA | 0 |
| PHP96716 | NOS | UBI | KN1 | HSP17--up | HRA | 1 |
| PHP96031 | GOS2 | UBI | | HSP17-- | HRA | 3 |
| | | | | down | | |
| PHP96730 | SWEET11 | UBI | | HSP17-- | HRA | 0 |
| | | | | down | | |
| PHP96731 | DIURNA | UBI | | HSP17-- | HRA | 0 |
| | L10 | | | down | | |
| PHP96032 | DIURNA | UBI | | HSP17-- | HRA | 1 |
| | L11 | | | down | | |
| PHP96425 | NOS | 3xENH- | AT-5-IV-7 1x* | ins-HSP17-- | HRA | 4 |
| | | UBI | | down | | |
| PHP96751 | NOS | 3xENH-UBI | AT-5-IV-2 1x* | ins-HSP17-- | HRA | 4 |
| | | | | down | | |
| PHP96919 | 3xEME- | 3xEME- | | HSP17-- | HRA | 3 |
| | NOS | UBI | | down | | |
| PHP97417 | UBI | 3xENH- | | ins-HSP17-- | HRA | 3 |
| | | UBI | | down | | |
| PHP97334 | NOS | 3xENH- | AT-5-IV-2 | ins-HSP17-- | NPTII-- | 4 |
| | | UBI | 2x** | down | down | |
| PHP97335 | NOS | 3xENH- | | ins-HSP17-- | NPTII-- | 2 |
| | | UBI | | down | up | |
| PHP97458 | FT- | 3xEME- | | HSP17-- | HRA | 1 |
| | MEM1- | UBI | | down | | |
| | nos | | | | | |
| PHP97725 | NOS- | 3xENH- | | ins-HSP17-- | HRA | 1 |
| | WUS-ins | UBI | | up | | |
| PHP97726 | NOS- | 3xENH- | | ins-HSP17-- | HRA | 1 |
| | WUS-ins | UBI | | up | | |
| PHP97933 | NOS | 3xENH- | AT-5-IV-2 | ins-HSP17-- | NPTII | 4 |
| | | UBI | 2x** | down | | |
| | | | CAS9 + gRNA *** | | | |
| PHP98784 | NOS | 3xENH- | AT-5-IV-2 | ins-HSP17-- | NPTII | 4 |
| | | UBI | 2x ** | down | | |
| | | | CAS9 + gRNA | | | |
| PHP98407 | | | UBI:ZM- | HSP17-- | HRA | 0 |
| | | | GRF5 | down | | |
| PHP98310 | NOS | | UBI:ZM- | HSP17-- | HRA | 0 |
| | | | GRF5 | down | | |
| PHP98248 | NOS | UBI | UBI:ZM- | HSP17-- | HRA | 1 |
| | | | GRF5 | down | | |
| PHP98283 | NOS | 3xENH- | UBI:ZM- | HSP17-- | HRA | 2 |
| | | UBI | GRF5 | down | | |
| PHP98393 | 3xENH- | 3xENH- | Excision- | HSP17-- | HRA | 1 |
| | UBI T2A | UBI | activate | down | | |
| | | | GRF5 | | | |
| PHP98392 | NOS | 3xENH- | UBI:GRF5 | HSP17-- | HRA | 2 |
| | | UBI | | down | | |
| PHP98567 | 3xEME- | 3xEME- | | HSP17-- | HRA | 2 |
| | UBI | UBI | | down | | |
| PHP97452 | 3xEME- | UBI | | HSP17-- | HRA | 1 |
| | UBI | | | down | | |
| PHP97456 | 3xEME- | UBI adh | | HSP17-- | HRA | 2 |
| | UBI | | | down | | |
| PHP97977 | 3xEME- | UBI TR | | HSP17-- | HRA | 1 |
| | UBI | | | down | | |
| PHP97453 | UBI Adh | 3xEME- | | HSP17-- | HRA | 2 |
| | | UBI | | down | | |
| PHP97449 | UBI TR | 3xEME- | | HSP17-- | HRA | 2 |
| | | UBI | | down | | |
| PHP98680 | 3xENH- | 3xENH- | Excision- | INS- | HRA | 2 |
| | UBI T2A | UBI T2A | activated | HSP17-- | | |
| | | | MIR156B | down | | |
| PHP98681 | UBI- | 3 XENH- | Excision- | INS- | HRA | 2 |
| | NOS- | UBI-BBM | activated | HSP17-- | | |
| | WUS | | MIR156B | down | | |
| PHP98328 | BDCAB2- | UBI | | HSP17-- | HRA | 1 |
| | 2xEME | | | down | | |
| PHP98329 | ZMGOS2- | UBI | | HSP17-- | HRA | 1 |
| | 2xEME | | | down | | |
| PHP98327 | BDCAB2- | 3xENH- | | HSP17-- | HRA | 2 |
| | 2xEME | UBI | | down | | |
| PHP98370 | ZM | 3xENH- | | HSP17-- | HRA | 2 |
| | GOS2- | UBI | | down | | |
| | 2xEME | | | | | |
| PHP98564 | PLTP | UBI | | HSP17-- | HRA | 2 |
| | | | | down | | |
| PHP98565 | PLTP | 3xENH- | | HSP17-- | HRA | 2 |
| | | UBI | | down | | |
| PHP96037 | NOS | 3xENH- | | HSP17-- | HRA | 3 |
| | | UBI | | down | | |
| PHP97447 | UBI | 3 XEME- | | HSP17-- | HRA | 1 |
| | | UBI | | UP | | |
| PHP97881 | UBI | 3 XEME- | | HSP17-- | HRA | 3 |
| | | UBI | | down | | |
| PHP97417 | UBI | 3xENH- | | HSP17-- | HRA | 3 |
| | | UBI | | down | | |
| PHP96037 | NOS | 3xENH- | | HSP17-- | HRA | 3 |
| | | UBI | | down | | |
| PHP81858 | NOS | UBI | | HSP17-- | HRA | 2 |
| | | | | down | | |
| PHP99676 | NOS | GRP1 | | HSP17-- | HRA | 0 |
| | | | | down | | |
| PHP99677 | NOS | RPL1 | | HSP17-- | HRA | 1 |
| | | | | down | | |
| PHP99678 | NOS | DNAJ | | HSP17-- | HRA | 0 |
| | | | | down | | |
| PHP99679 | NOS | SAMDC2 | | HSP17-- | HRA | 0 |
| | | | | down | | |
| PHP99680 | NOS | PPISO | | HSP17-- | HRA | 0 |
| | | | | down | | |
| PHP99569 | NOS | EF1A | | HSP17-- | HRA | 0 |
| | | | | down | | |
| PHP96037 | NOS | 3xENH- | | HSP17-- | HRA | 3 |
| | | UBI | | down | | |
| PHP100011 | NOS | 3xENH- | | HSP17-- | HRA | 1 |
| | | RPL1 | | down | | |
| PHP100012 | NOS | 3xENH- | | HSP17-- | HRA | 0 |
| | | DNAJ | | down | | |
| PHP100013 | NOS | 3xENH- | | HSP17-- | HRA | 1 |
| | | SAMDC2 | | down | | |
| PHP100056 | NOS | 3xENH- | | HSP17-- | HRA | 1 |
| | | ZMPPISO | | down | | |
| PHP100057 | NOS | 3xENH- | | HSP17-- | HRA | 1 |
| | | ZMEF1A | | down | | |
| PHP100158 | NOS | 3xENH- | | HSP17-- | HRA | 1 |
| | | GRP1 | | down | | |
| PHP98229 | NOS | 3xENH- | | HSP17-- | HRA | 3 |
| | | UBI:BBM | | down | | |
| PHP100159 | NOS | 3xENH- | UBI:CYCD2 | HSP17-- | HRA | 2 |
| | | UBI | | down | | |
| PHP100160 | NOS | 3xENH- | UBI:REPA | HSP17-- | HRA | 2 |
| | | UBI | | down | | |
| PHP100229 | ATPSYN | 3xENH- | | HSP17-- | HRA | 2 |
| | | UBI | | down | | |
| PHP99971 | EIF4A | 3xENH- | | HSP17-- | HRA | 2 |
| | | UBI | | down | | |
| PHP99809 | PABP | 3xENH- | | HSP17-- | HRA | 2 |
| | | UBI | | down | | |
| PHP99810 | VDAC1A | 3xENH- | | HSP17-- | HRA | 2 |
| | | UBI | | down | | |
| PHP99716 | LEA14 | 3xENH- | | HSP17-- | HRA | 2 |
| | | UBI | | down | | |

*Single Cross-Talk Blocker sequence upstream (5′) of the HSP17 promoter
**Two Cross-Talk Blocker sequences, one upstream and one downstream flanking the HSP17::CRE expression cassette
***CRE-mediated excision of Cas9 and gRNA

### B. Configurations of WUS/BBM Expression Cassettes

A comparison of the following is performed:

### i) PHP35648 (SEQ ID NO:1):UBI::CYAN+RAB17::CRE+NOS::WUS2+UBI::ODP2

The Ubiquitin (UBI) promoter from maize is a strong constitutive promoter, while the nopaline synthase (NOS) promoter derived from Agrobacterium is a constitutive promoter which in maize drives expression at approximately a 20% level compared to UBI. However, strong expression cassettes upstream of NOS::WUS (such as UBI::CYAN and RAB17::CRE) have the potential to down-regulate WUS expression, compared to a T-DNA where the strong upstream expression cassettes have been removed.

When maize seedling-derived leaf segments are transformed using Agrobacterium strain LBA4404 TD THY- and a T-DNA-containing plasmid with UBI::CYAN+RAB17::CRE+NOS::WUS2+UBI::ODP2 (PHP35648), expression of WUS2 and ODP2 is observed to slow initiation and growth of callus, which becomes increasingly embryogenic over time. Using this construct, 10-12 weeks of callus growth is required before RAB17::CRE-mediated excision and subsequent somatic embryo maturation and T0 plant regeneration (Assay Score=1).

### ii) PHP81858 (SEQ ID NO:8): NOS::WUS2+UBI::ODP2+RAB17::CRE

When maize seedling-derived leaf segments were transformed using Agrobacterium strain LBA4404 TD THY- and PHP81858, in which the upstream strong expression cassettes were not present, the combination of NOS::WUS2+UBI::ODP2 resulted in a moderate rate of embryogenic callus growth, with a higher percentage of leaf segments/pieces producing positive responses. Using this construct, 6-8 weeks of callus growth was required before RAB17::CRE-mediated excision and subsequent somatic embryo maturation and T0 plant regeneration (Assay

iii) PHP95385 (SEQ ID NO:47): ACTIN::WUS+UBI::ODP2+HSP::CRE

When maize seedling-derived leaf segments were transformed using Agrobacterium strain LBA4404 TD THY- and PHP95385 containing ACTIN PRO::WUS2+UBI PRO::ODP2+HSP17 PRO::CRE resulted in a moderate rate of embryogenic callus growth, with a higher percentage of leaf segments/pieces producing positive responses. Using this construct, 6-8 weeks of callus growth was required before CRE-mediated excision and subsequent somatic embryo maturation and T0 plant regeneration (Assay Score=2).

### iv) PHP81856 (SEQ ID NO:6): AXIG1::WUS2+PLTP::ODP2+RAB17::CRE

In contrast to constitutive promoters NOS and UBI, the maize AXIG1 promoter is induced by the presence of auxin in the medium and is generally about 20% as strong as the maize UBI promoter (in the presence of our standard concentrations of 2,4-D). The PLTP promoter appeared to be strong relative to UBI but expression of the PLTP promoter is not as constitutive as the UBI promoter. When PHP81856, AXIG1::WUS2+PLTP::ODP2, was used for Agrobacterium-mediated transformation, in immature embryos and in leaf segments similar levels of transient ZS-GREEN expression were observed indicating that T-DNA delivery occurred at an equivalent extent in both explants. However, the subsequent growth response from these two explants was different. In immature embryos, expression of AXIG1::WUS2+PLTP::ODP2 resulted in rapid somatic embryo formation. In contrast, when AXIG1::WUS2+PLTP::ODP2 was used in leaf segments, no growth of transgenic (green fluorescent) callus or somatic embryos occurred and no T0 plants were recovered because expression of WUS2 and ODP2 did not continue for a long enough duration (Assay Score=0).

### v) PHP96037 (SEQ ID NO:66): NOS::WUS2+3×ENH::UBI::ODP2

When maize seedling-derived leaf segments were transformed using Agrobacterium strain LBA4404 TD THY- and PHP96037, containing NOS::WUS2+3×ENH::UBI PRO::UBI::ODP2+HSP17 PRO::CRE, somatic embryos formed rapidly, emerging directly from the leaf segments with no intervening callus stage. Direct somatic embryo formation was observed between 10-14 days after Agrobacterium infection. Thus, the strength and longer duration of WUS2 and ODP2 expression provided by PHP96037 was sufficient to stimulate rapid somatic embryo formation. Using this construct, only 4-6 weeks of callus growth was required before CRE-mediated excision and subsequent somatic embryo maturation and T0 plant regeneration (Assay Score=4).

C. Testing New Promoters Driving Expression of WUS2 and/or ODP2

The results from experiments such as those summarized in Table 18 clearly demonstrated that strong constitutive promoters such as the maize UBI1ZM PRO (or enhanced versions of UBI1ZM PRO) driving expression of ODP2 in conjunction with various additional helpers, excision components, and selectable markers effectively stimulated rapid somatic embryo formation and T0 plant regeneration, while a range of constitutive promoters such as GOS2 or NOS (both around 15-20% as strong as UBI1ZM) up to the UBI PRO itself, and including the ACTIN PRO, the 8×DR5-35S PRO, and the FT-MEM1-NOS PRO when used for driving WUS2 expression in conjunction with various additional helpers, excision components, and selectable markers were effective to stimulate rapid somatic embryo formation and T0 plant regeneration. New promoter candidates were identified to be used in conjunction with various additional helpers, excision components, and selectable markers, resulting in the lists shown in Tables 198 and 2019.

To test these potential promoter candidates, T-DNAs with the following configurations are constructed:

$$R B + P R O - 1 \text{::} W U S 1 + 3 x E N H \text{::} U B I 1 Z M \text{::} O D P 2 + U B I \text{::} Z S - GREEN + U B I \text{::} N P T I I + L B ; \qquad Configuration 1.$$

$$R B + N O S \text{::} W U S 1 + 3 x E N H \text{::} P R O - 2 \text{::} O D P 2 + U B I \text{::} Z S - GREEN + U B I \text{::} N P T I I + L B ; and \qquad Configuration 2.$$

$$R B + N O S \text{::} W U S 1 + P R O - 2 \text{::} O D P 2 + U B I \text{::} Z S - GREEN + U B I \text{::} N P T I I + L B . \qquad Configuration 3.$$

Based on the experimental observations herein, the promoters in Table 19 are expected to produce positive results (Assay Scores of “2-4”) when used in the “PRO-1” position in Configuration 1 above to drive expression of WUS2. Promoters indicated in Table 19 by a single asterisk are expected to produce rapid embryogenic growth (scores of 2-4) when substituted for PRO-2 in Configuration 2, and promoters indicated by a double asterisk are expected to produce rapid embryo formation in Configurations 2 or 3. Likewise, the six new promoters listed in Table 20 are expected to perform equal to or better than UBI1ZM when substituted in Configurations 2 and 3 (driving expression of ODP2).

TABLE 19

| SEQ | Promoter | |
|---|---|---|
| ID NO: | Alias | Gene Description |
| 94 | ATPeF1D ** | ATP synthase, delta/epsilon chain |
| 95 | EIF4a ** | EIF4a translation initiation factor |
| 96 | RRM ** | RNA Recognition Motif gene |
| 97 | EF1A * | Translation elongation factor EF1A/initiation |
| 98 | RPL10A * | Ribosomal protein L10A |
| 99 | AXP2 ** | Ascorbate peroxidase2 |
| 100 | VDAC1a ** | Voltage-dependent anion channella |
| 101 | EF1A-Tu * | Elongation factor Tu GTP binding domain |
| | | protein |
| 102 | LEA-14 ** | Late embryogenesis abundant protein, LEA-14 |
| 103 | RP-S7 * | Ribosomal protein S7 |
| 104 | RP-L5 * | Ribosomal protein L5 |
| 105 | ENO2 * | Enolase2 |
| 106 | RP-L28 * | Ribosomal protein L28 |
| 107 | OS-ACTIN ** | Actin |
| 108 | ZM-UBI2 ** | Ubiquitin2 |
| 109 | UBIIZM * | Ubiquitin1 |

* Promoters being tested for WUS (Configuration 1) and/or ODP2 (Configuration 2)
** Promoters being tested for ODP2 in Configurations 2 and 3 above.

TABLE 20

| SEQ | | |
|---|---|---|
| ID NO: | Gene_Alias | Description |
| 109 | UBIIZM | Polyubiquitin containing 7 ubiquitin monomers |
| 110 | GRP1 | glycine-rich protein1 |
| 111 | RP-L1 | Ribosomal protein L1, 3-layer alpha/beta- |
| | | sandwich |
| 112 | DNAJ2 | chaperone DNA J2 5NOC3 |
| 113 | SAMDC2 | S-adenosyl methionine decarboxylase2 |
| 114 | CPPIase | Cyclophilin type peptidyl-prolyl cis-trans |
| | | isomerase |

### Example 8: Seedling Pre-Treatment with Ancymidol Improved Transformation

A. Seedling Pre-Treatment with Ancymidol Improved Agrobacterium-Mediated Random Transformation

Methods for Agrobacterium-mediated transformation of maize leaf segments/tissue were followed as outlined in Examples 4 and 5. Specifically, seed of Pioneer inbred PHH5E was surface sterilized and sown on germination medium containing either no ancymidol (0 mg/l ancymidaol=control medium 900 medium), 2 mg/l ancymidol (70AE medium), or 4 mg/l ancymidol. The germination and growth period under 120 μmol m-2 s-1 light intensity using an 18-hour photoperiod at 28° C. was 14 days for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 and PHP97334 (SEQ ID NO:4 and SEQ ID NO:77).

Table 21 shows that growing seedlings on ancymidol improved subsequent leaf transformation. For the third experiment, the control treatment was not performed, but the general trend in these experiments is nonetheless clear; seedlings grown on both levels of ancymidol produced higher leaf transformation frequencies. Looking at the average results summarized at the bottom of Table 21, seedlings grown on either 2 mg/l or 4 mg/l ancymidol showed higher Agrobacterium-mediated T-DNA than the control (with delivery scores of 4 for both ancymidol treatments compared to the control value of 3). Ancymidol-grown seedlings also resulted in higher overall rates of early somatic embryo formation for both levels of the hormone, with leaf segments from 2 mg/l and 4 mg/l ancymidol treatments having more rapidly forming somatic embryos, higher numbers of somatic embryos per leaf segment, and an overall higher percentage of leaf segments producing somatic embryos relative to the control treatment.

The higher T-DNA delivery and a more robust somatic embryo formation observed in the ancymidol treatments were reflected in higher transformation frequencies (Txn %, calculated based on the number of transgenic T0 plants per starting seedling) from seedlings grown on ancymidol, with seedlings grown on 2 mg/l or 4 mg/l ancymidol producing an average T0 Txn % that was 3-fold or 2.5-fold higher than the control treatment, respectively. Despite greater T-DNA delivery and T0 Txn % for ancymidol-grown seedlings, the transgenic T0 plants produced in all three treatments showed similar single-copy T-DNA frequencies as measured by qPCR for the transgenes within the T-DNA, demonstrating that the increased numbers of transgenic TO plants produced from ancymidol-grown seedlings was not accompanied by an increase in integrated T-DNA copy number.

TABLE 21

| Exp. | Ancymidol | No. | T-DNA | No. T0 | | # SC | |
|---|---|---|---|---|---|---|---|
| No. | Conc. | Seedlings | Delivery* | Plants | Txn %** | T0s*** | SC % |
| 1 | 2 mg/l | 33 | 4 | 118 | 358% | 51 | 43% |
| | 4 mg/l | 33 | 4 | 83 | 252% | 45 | 54% |
| | 0 mg/l | 33 | 3 | 36 | 109% | 19 | 53% |
| 2 | 2 mg/l | 32 | 4 | 97 | 303% | 67 | 69% |
| | 4 mg/l | 32 | 4 | 81 | 253% | 51 | 63% |
| | 0 mg/l | 32 | 3 | 31 | 97% | 19 | 61% |
| 3 | 2 mg/l | 28 | 4 | 66 | 236% | 27 | 41% |
| | 4 mg/l | 32 | 4 | 75 | 234% | 51 | 68% |
| | 0 mg/l | 0 | n/a | n/a | n/a | n/a | n/a |
| Totals | 2 mg/l | 93 | 4 | 281 | 302% | 145 | 52% |
| | 4 mg/l | 97 | 4 | 239 | 246% | 147 | 62% |
| | 0 mg/l | 65 | 3 | 67 | 103% | 38 | 57% |

*Txn % = Transformation Frequency (%)
**Relative T-DNA delivery as assessed by transient ZS-GREEN expression (0 = None to 4 = almost all embryos expressing)
**SC = single copy of the integrated T-DNA

Both 2% and 1% ancymidol pre-treatment during seed germination and seedling growth were also tested for the cereals (using medium 900 plus 1 mg/l or 2 mg/l ancymidol). For Japonica rice (Oryza sativa var Kitaake), teff (Eragrostis tef), and pearl millet (Pennisetum glaucum), seedling growth on medium 900 without additional ancymidol resulted in very thin elongated seedlings with little biomass due to the thin leaf-whorl region above the mesocotyl. When seed from all three species were germinated and grown on 900 medium plus 2 mg/l ancymidol, the seedlings only grew to a height of 1-2 cm after 14 days and although the leaf-whorl region was thicker (due to wider leaves), processing these small seedlings to produce leaf segments/pieces followed by transformation was more difficult.

In contrast, for all three cereal crops, seed germination and seedling growth on 900 plus 1 mg/l ancymidol produced an intermediate growth rate, with thicker stems and wider leaves than the control (with no ancymidol). These whorl segments were readily processed in a food processor to produce appropriately sized leaf fragments/segments, showed good Agrobacterium-mediated T-DNA delivery with abundant transient ZS-GREEN expression, and produced the highest number of transgenic T0 plantlets (compared to the other two treatments). Thus, compared to maize and sorghum in which 2 mg/l ancymidol pre-treatment during seedling growth is optimal for leaf transformation, a lower concentration of 1 mg/l ancymidol produced optimal results in rice, tef, and pearl millet.

B. Seedling Pre-Treatment with Ancymidol Improved Agrobacterium-Mediated Site-Specific Integration

A pre-integrated target site (target locus) in the maize inbred HC69 genome was used for site-specific integration, as described in U.S. Pat. Nos. 6,187,994, 6,262,341, 6,330,545, 6,331,661, and 8,586,361, each of which is herein incorporated by reference in its entirety. In this example, target site 45 located on chromosome 1 at genetic position 53.66 cM within the PHH5E inbred genome was used and is comprised of the integrated components loxP+UBI1ZM PRO::UBI1ZM 5′UTR::UBI1ZM INTRON1::FRT1::NPTII::PINII TERM+FRT6 which had been previously introduced via Cas9-mediated homologous recombination to create this SSI landing pad. Seed was surface sterilized, germinated on 90AE medium (900 medium+2 mg/l ancymidol), and leaf segments were prepared from 14 day-old seedlings using a food processor and 100 ml of Agrobacterium suspension. The Agrobacterium strain LBA4404 TD THY-contained the helper plasmid PHP71539 in addition to the T-DNA-containing plasmid PHP90842 (SEQ ID NO:14; T-DNA with RB+UBI::FLP+FRT1::PMI+ACTIN::WUS+UBI::ODP2+HSP17.7::CRE+LOXP+SB-UBI::DsRED2+FRT6+LB) and the second strain also contained PHP93925 (SEQ ID NO:26; T-DNA with RB+UBI::WUS+3×ENH::UBI::ODP2+SB-UBI::ZS-GREEN+HRA+LB) at a ratio of 9:1 (PHP90842: PHP93925). The OD of both constructs was 0.4 at 550 nm. Surfactant solution (Break-Thru S 233) was diluted by adding sterile ddH₂O to a produce a stock 10% concentration, and then adding the 10% surfactant solution to the Agrobacterium suspension to give a final concentration 0.02% (v/v).

Leaf tissue was processed by first dissecting out the 3 cm of whorl tissue immediately above the mesocotyl and placing it in a food processor along with 100 ml of the mixed Agrobacterium suspension in 700J medium plus acetosyringone. Short 1-2 second pulses were administered until the leaf segments/fragments were approximately 2-3 mm in size, and then the mixture (leaf pieces/segments and Agrobacterium mix suspended in infection medium) was allowed to sit for 15 minutes in the blender. After 15 minutes of infection, the leaf segments/tissue was separated from the liquid by pouring through a stainless-steel sieve, and then the leaf segments/tissue was transferred to glass filter paper supports resting within 60×25 mm plates. The leaf tissue pieces/segments resting on the dry filer papers were allowed to stand for few minutes and then the filter paper (supporting the leaf segments/pieces) was transferred onto co-cultivation medium. The tissue pieces/segments were then spread evenly across the filter using a sterile inoculation loop. Co-cultivation on 710N medium was done at 21° C. in the dark for 2 days, at which point the leaf segments/pieces were transferred to resting medium 605B (using forceps to lift and transfer the entire filter) and incubated at 28° C. in the dark for 14 days. At the end of the resting period, the filters were moved onto selection medium (6050=605J medium with sucrose removed and 15 g/l mannose added) and incubated at 28° C. in the dark, with transfers to fresh 6050 medium every two weeks. After 6 weeks on selection, the plates with filters and leaf segments/tissue were transferred to a 45° C./70% RH incubator for 2 hours, allowing this heat shock treatment to activate the HSP17.7 PRO::CRE expression cassette. After 2 hours in the heat treatment, plates were transferred back into the hood and allowed to cool to room temperature. The segments/tissue was then picked off the filters using forceps and transferred to maturation medium (13329B) for 18 days at 28° C. in the dark, and the plates were then moved into a culture room set at 26° C. with dim light. Healthy shoots were then selected and transferred to 272M (272X with 10 mg/l meropenem) rooting medium for an additional 2-3 weeks at 26° C. with light, before being transferred to the greenhouse.

As shown in Table 22, using the above method resulted in successful Site-Specific Integration. Starting with 30 seedlings to prepare the target tissue/segments for Agrobacterium-mediated transformation, 127 leaf pieces/segments responded by producing somatic embryos. From this initial growth response, 44 embryogenic calli continued to grow on mannose-containing selection medium. From this number of calli, seven regenerated into T0 plants, of which site-specific integration was confirmed in 4 plants by molecular analysis and one of these 4 events had perfectly recombined junctions at both ends of the double-recombination product. This event, labeled as RMCE in Table 22, also contained no T-DNA sequences including no indications of FLP, WUS2, ODP2 or Agrobacterium backbone.

TABLE 22

| | Number of: | | | | | |
|---|---|---|---|---|---|---|
| | Leaf | | | | | |
| | segments/pieces | Putative | Events | RMCE | SSI | |
| Seedlings | responded | events | regenerated | events | events | Escapes |
| 30 | 127 | 44 | 7 | 1 | 3 | 3 |

Any one of the compounds listed in Table 23 in appropriate concentrations for pre-treatment of seedlings produces leaf segments that exhibit an improvement in frequencies of T-DNA delivery, somatic embryo response (more rapid growth and higher numbers), production of T0 plants, and single-copy integration frequencies.

TABLE 23

| Category | Chemical or Physical Treatment |
|---|---|
| Auxins | 2,4-D, dicamba, 2,4,5-T, 1-Naphthaleneacetic acid (NAA), picloram, |
| | indole-3-acetic acid, 4-chloroindole-3-acetic acid, phenylacetic acid, |
| | indole-3-butyric acid, and indole-3-propionic acid |
| Anti-oxidants | dithiolthreitol (DTT), cysteine, glutathione |
| Gibberellic Acid | ancymidol, paclobutrazol, uniconazole, chlormequat chloride, mepiquat, |
| Inhibitors | AMO-1618 [(2′-isopropyl-4′-(trimethylammoniumchloride)-5′- |
| | methylphenylpiperidinecarboxylate)], clorphonium-Cl, tetcylacis, |
| | flurprimidol, inabenfide, pro-hexadione, trinexapac-ethyl, daminozide, |
| | exo-16,17-acetate, malic hydrazide, phoshor D, alar, chlorocholine |
| | chloride, cycocel, the fungicides tebuconazole and metconazole, |
| | trinexapac-ethyl, prohexadione-calcium, exo-16,17-dihydro-GA5-13- |
| | acetate |
| Inhibit Abscisic | norflurazon, fluridone, diflufenican, abamine, nordihydroguaiaretic acid |
| Acid Biosynthesis | (NDGA) |
| Abscisic Acid | AA1 [1-[(4-benzylpiperazin-1-yl)methyl]-2-sulfanylidene- |
| Antagonist | 1H,2H,5H,6H,7H,8H-[1,3,4]thiadiazolo[3,2-a][1,3]diazepin-5-one], AS6 |
| | [3′-hexylsulfanyl-ABA], DFPM([5-(3,4-Dichlorophenyl)furan-2-yl]- |
| | piperidin-1-ylmethanethione), RK460 |
| Low Abscisic Acid | 10⁻⁶ to 10⁻⁷ M |
| Concentrations | |
| Abscisic Acid | pyrobactin |
| agonist | |
| Cytokinin | BAP [6-benylamino purine], BA [benzyladenine], kinetin, TDZ |
| | [thidiazuron], trans-zeatin |
| Anti-cytokinin | S-4893 [a 4-phenylquinazoline derivative], 4-phenylquinazoline, 6-(2,5- |
| | Dihydroxybenzylamino)purine (LGR-991), 2-hydroxy-3-Methyl- |
| | benzyladenine |
| Cyclin-Dependent | caffeine |
| Kinase (CDK) | |
| inhibitor | |
| Auxin agonists | quinclorac, RubNeddins (RN1, RN2, RN3, and RN4) |
| Auxin antagonists | p-Chlorophenoxy isobutylic (PCIB), tert-butoxycarbonylaminohexyl- |
| | IAA (BH-Indole Acetic Acid = BH-IAA), α-(phenylethyl-2-oxo)-IAA |
| | (PEO-IAA), α-(2,4-dimethylphenylethyl-2-oxo)-IAA (auxinole) |
| Auxin/Cytokinin- | fipexide |
| like | |
| Methylation | 5-azacytidine, Decitabine, RG108 [N-Phthalyl-L-tryptophan], |
| inhibitors | zebularine, thioguanine, 2′-deoxy-5-fluoreocytidine, CM-272 (6- |
| | methoxy-2-(5-methyl-2-furyl)-N-(1-methyl-4-piperidyl)- |
| | 7-(3-pyrrolidin-1-ylpropoxy)quinolin-4-amine), CM-579 (6-methoxy-2- |
| | (5-methyl-2-furyl)-N-[(1-methyl-4-piperidyl)methyl]-7-(3-pyrrolidin-1- |
| | ylpropoxy)quinolin-4-amine), SGI-1027 [N-[4-[(2-Amino-6-methyl-4- |
| | pyrimidinyl)amino]phenyl]-4-(4-quinolinylamino)benzamide], gamma- |
| | oryzanol, and (−)-epigallocatechin gallate (ECGC), β-Thujaplicin, |
| | procainamide |
| Histone deacetylase | ~70 candidates in literature (Cancer research), TSA, valproic acid, |
| (HDAC) Inhibitors | tacedinaline, cambinol |
| Pan-HDAC | trichostain A (TSA), verinostat, panobinostat, belinostat |
| Inhibitors | |
| HDAC3 Inhibitor | RGFP-966 [(E)-N-(2-amino-4-fluorophenyl)-3-(1-cinnamyl-1H-pyrazol- |
| | 4-yl)acrylamide] |
| HDCA6 Inhibitor | tubastatin A |
| HDAC8 Inhibitor | PCI-34051 [N-Hydroxy-1-[(4-methoxyphenyl)methyl]-1H-indole-6- |
| | carboxamidel |
| Sugar composition | Sucrose, Maltose |
| Metals | Copper, Zinc, Manganese [MnSO4] |
| Amino | proline, asparagine, methionine, glutamine |
| acids/specific ions | |
| Heat | 45° C. for 2-3 hours, 37° C. overnight |
| Cold shock | Below standard incubation temperature |
| Ethylene inhibitor | silver nitrate, 2-aminoethoxyvinyl glycine (AVG), silver ions (Ag), and |
| | the gaseous compound 1-methylcyclopropene (1-MCP) |
| Ethylene | norbornadiene |
| competitive inhibitor | |
| Ethylene | ethylene absorption bags |
| sequestration | |
| light treatments | Valoya and Razor3 LED light arrays |
| 70° F. day/65° F. night | regular Greenhouse temp is 70° F.-75° F. |
| Polycomb | EPZ005687 [1-cyclopentyl-N-[(4,6-dimethyl-2-oxo-1H-pyridin-3- |
| Repressive Complex | yl)methyl]-6-[4-(morpholin-4-ylmethyl)phenyl]indazole-4-carboxamide], |
| 2 (PRC2) inhibitors | UNC1999 [N-[(6-methyl-2-oxo-4-propyl-1H-pyridin-3-yl)methyl]-1- |
| | propan-2-yl-6-[6-(4-propan-2-ylpiperazin-1-yl)pyridin-3-yl]indazole-4- |
| | carboxamide], PF-06726304 [C₂₂H₂₁Cl₂N₃O₃], Lirametostat, |
| | Tazemetostat, EPZ011989 (C₃₅H₅₁N₅O₄•HCl), CPI-169 (C₂₇H₃₆N₄O₅S), |
| | and JQ-EZ-05 (C₃₀H₃₈N₈O₂) |
| Jasmonates | Jasmonic acid, Methyl-jasmonate |
| Safeners | naphthalic anhydride, salicylic acid, fenchlorazole, cloquintocet-mexyl, |
| | mefenpyr diethyl |
| Kinase inhibitors | N-[(2R)-2,3 dihydroxypropoxy]-3,4-difluoro-2- (2-fluoro-4-iodoanilino) |
| | benzamide, anthra (1,9-cd) pyrazol-6 (2H)-one: 4-(4-Fluoro phenyl)-2- |
| | (4-methylsulfinylphenyl)-5-(4-pyridyl) 1H imidazole, N-benzyl-2- |
| | (pyrimidin-4-ylamino)-1,3 thiazole-4-carboxamide |
| reactive oxygen | Tiron, dimethylthiourea (DMTU), diphenyleneiodonium (DPI) |
| species (ROS) | |
| scavengers | |

### Example 9: Other Gibberellic Acid Inhibitor Pre-Treatment Prior to Agrobacterium Infection Improved Transformation

Maize leaf segments/tissue were pre-treated with one of three other gibberellic acid inhibitors (GAI) listed in Table 23, paclobutrazol (PBZ), chlormequat chloride, or daminozide prior to Agrobacterium infection to determine their effects on transformation success.

Methods for Agrobacterium-mediated transformation of maize leaf segments/tissue were followed as outlined in Examples 4 and 5. Specifically, seed of Pioneer inbred ED85E was surface sterilized and sown on 900 (PBZ) or 90AO (chlormequat chloride and daminozide) germination medium containing either:
a) 0 mg/1, 2 mg/l, 4 mg/l, or 6 mg/l of PBZ;
b) 0 mg/l, 250 mg/l, or 500 mg/l of chlormequat chloride; or
c) 0 mg/l, 50 mg/1, 100 mg/l, or 150 mg/l of daminozide.
   Each of the three GA inhibitors were test individually. 90AO medium contained 900 medium+1 mg/l ancymidol. The germination and growth period under 120 μmol m-2 s-1 light intensity using an 18-hour photoperiod at 28° C. was 13 to 16 days for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 (SEQ ID NO:4) plus either PHP101093 (SEQ ID NO:351) for PBZ experiments or PHP97334 (SEQ ID NO:77) for chlormequat chloride and daminozide experiments.

Pre-treating seedlings with chlormequat chloride had a positive impact on maize leaf transformation (Table 24). Using 250 mg/l chlormequat chloride for pretreatment of ED85E seedlings improved T0 transformation frequency (Txn %) by 1.4-fold relative to the control with no GA inhibitor in the germination medium.

For paclobutrazol and daminozide, where no clear trend was observed in the data, testing of higher concentration ranges for paclobutrazol (10, 20, 50 mg/l) and lower concentration for daminozide (20, 10, 3, 1, and 0.3 mg/l) is observed to increase T0 transformation frequencies relative to T0 transformation frequencies in the control treatment where just the basal germination medium is used to grow seedlings. Table 24 shows the results of the GA inhibitor pre-treatment experiments. PTRT=pre-treatment, TRT=treatment.

TABLE 24

| | | | | T-DNA | # culture | # culture | | |
|---|---|---|---|---|---|---|---|---|
| Seedling | PTRT conc | # | | score | response | response | # T0 | TXN |
| PTRT | (mg/l) | Seedling | TRT | (0-5) | (2 wk) | (4 wk) | plants | % |
| PBZ | 0 | 24 | 1 | 4 | 252 | 281 | 88 | 367 |
| PBZ | 2 | 24 | 2 | 3.5 | 271 | 224 | 60 | 250 |
| PBZ | 4 | 24 | 3 | 3.5 | 212 | 156 | 40 | 167 |
| PBZ | 6 | 24 | 4 | 3.5 | 151 | 99 | 29 | 121 |
| Chlormequat | 0 | 30 | 1 | 4 | 283 | 315 | 103 | 343 |
| chloride | | | | | | | | |
| Chlormequat | 250 | 30 | 2 | 4.5 | 265 | 318 | 145 | 483 |
| chloride | | | | | | | | |
| Chlormequat | 500 | 30 | 3 | 4.5 | 302 | 339 | 108 | 360 |
| chloride | | | | | | | | |
| Daminozide | 0 | 20 | 1 | 4 | 165 | 200 | 109 | 545 |
| Daminozide | 50 | 20 | 2 | 4 | 87 | 99 | 71 | 355 |
| Daminozide | 100 | 20 | 3 | 4 | 103 | 103 | 41 | 205 |
| Daminozide | 150 | 20 | 4 | 4 | 70 | 95 | 53 | 265 |

When compared to control seedlings, seedlings pre-treated with a GAI, including but not limited to GAIs listed in Table 23 (ancymidol, paclobutrazol, uniconazole, chlormequat chloride, mepiquat, AMO-1618 [(2′-isopropyl-4′-(trimethylammoniumchloride)-5′-methylphenylpiperidinecarboxylate)], clorphonium-Cl, tetcylacis, flurprimidol, inabenfide, pro-hexadione, trinexapac-ethyl, daminozide, exo-16,17-acetate, malic hydrazide, phoshor D, alar, chlorocholine chloride, cycocel, the fungicides tebuconazole and metconazole, trinexapac-ethyl, prohexadione-calcium, exo-16,17-dihydro-GA5-13-acetate), are observed to produce leaf segments that exhibit an improvement in frequencies of T-DNA delivery, somatic embryo response (more rapid growth and higher numbers), production of T0 plants, and single-copy integration frequencies.

### Example 10: Cytokinin Pre-Treatment Prior to Agrobacterium Infection Improved Transformation

Maize leaf segments/tissue were pre-treated with one of three cytokinins, thidiazuron (TDZ), 6-benylamino purine (BAP), or trans-zeatin prior to Agrobacterium infection to determine their effects on transformation success.

Methods for Agrobacterium-mediated transformation of maize leaf segments/tissue were followed as outlined in Examples 4 and 5. Specifically, seed of Pioneer inbred ED85E was surface sterilized and sown on 90AE germination medium containing either:
a) 0 mg/l, 0.1 mg/l, or 0.5 mg/l of TDZ;
b) 0 mg/l, 1 mg/l, or 5 mg/l of BAP; or
c) 0 mg/l, 1 mg/l, or 3 mg/l of trans-zeatin.
   Each of the three GA inhibitors were test individually. 90AE medium contained 90O medium+2 mg/l ancymidol. The germination and growth period under 120 μmol m-2 s-1 light intensity using an 18-hour photoperiod at 28° C. was 24 hours for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 (SEQ ID NO:4) plus PHP97334 (SEQ ID NO:77).

Pre-treating seedlings with BAP or trans-zeatin had a positive impact on maize leaf transformation (Table 25). Results show that using either 1 or 5 mg/l BAP increased T0 Txn % by 1.4-fold and 1.8-fold (respectively) relative to the control seedling germination medium with no cytokinin. Using either 1 or 3 mg/l trans-zeatin also increased T0 Txn % by 1.2-fold and 1.5-fold (respectively) relative to the control seedling germination medium with no cytokinin.

The use of TDZ in seedling pre-treatments resulted in no increase over the control treatment using 0.1 and 0.5 mg/l. Testing of higher concentration ranges, such as 1, 2, 5, or 10 mg/l TDZ is observed to produce an increase in somatic embryo response and an increase in transformation frequency (Txn %).

Table 25 shows the results of the cytokinin pre-treatment experiments. PTRT=pre-treatment, TRT=treatment.

TABLE 25

| | PTRT | | | T-DNA | # culture | # culture | | |
|---|---|---|---|---|---|---|---|---|
| Seedling | conc | # | | score | response | response | # T0 | |
| PTRT | (mg/l) | Seedling | TRT | (0-5) | (2 wk) | (4 wk) | plants | TXN % |
| TDZ | 0 | 21 | 1 | 4 | 180 | 122 | 103 | 490% |
| TDZ | 0.1 | 21 | 2 | 4 | 169 | 121 | 99 | 471% |
| TDZ | 0.5 | 21 | 3 | 4 | 182 | 122 | 97 | 462% |
| BAP | 0 | 21 | 1 | 4 | 91 | 135 | 70 | 333% |
| BAP | 1 | 21 | 2 | 4 | 135 | 151 | 97 | 462% |
| BAP | 5 | 21 | 3 | 4 | 144 | 172 | 128 | 610% |
| Trans- | 0 | 21 | 1 | 4 | 149 | 144 | 86 | 410% |
| Zeatin | | | | | | | | |
| Trans- | 1 | 21 | 2 | 4 | 196 | 177 | 102 | 486% |
| Zeatin | | | | | | | | |
| Trans- | 3 | 21 | 3 | 4 | 192 | 222 | 130 | 619% |
| Zeatin | | | | | | | | |

When compared to control seedlings, seedlings pre-treated with a cytokinin, including but not limited to cytokinins listed in Table 23 (BAP [6-benylamino purine], BA [benzyladenine], kinetin, TDZ [thidiazuron], trans-zeatin), are observed to produce leaf segments that exhibit an improvement in frequencies of T-DNA delivery, somatic embryo response (more rapid growth and higher numbers), production of T0 plants, and single-copy integration frequencies.

### Example 11: Fipexide Pre-Treatment Prior to Agrobacterium Infection Improved Transformation

Maize leaf segments/tissue were pre-treated with an auxin/cytokinin-like chemical, fipexide, prior to Agrobacterium infection to determine its effect on transformation success.

Methods for Agrobacterium-mediated transformation of maize leaf segments/tissue were followed as outlined in Examples 4 and 5. Specifically, seed of Pioneer inbred GR0112 was surface sterilized and sown on 90AE germination medium containing 0 mg/1, 4.75 mg/1, 9.5 mg/l, or 19 mg/l of fipexide. 90AE medium contained 900 medium+2 mg/l ancymidol. The germination and growth period under 120 μmol m-2 s-1 light intensity using an 18-hour photoperiod at 28° C. was 13 to 16 days for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 (SEQ ID NO:4) plus PHP96277 (SEQ ID NO:67).

Pre-treating seedlings with fipexide had a positive impact on maize leaf transformation (Table 26). Use of fipexide in the medium during germination and growth of GR0112 seedlings improved early somatic embryo formation (total number of somatic embryos (SE) formed per 42 starting seedings) within the first four weeks of culture after Agrobacterium infection, with use of 4.75, 9.5 and 19 mg/l of fipexide increasing the total SE number 1.4-fold, 1.3-fold, and 1.2-fold, relative to the control treatment where the seedling germination medium contains no fipexide. Regeneration of the somatic embryo tissue is performed using art recognized procedures (see Example 14C). The higher 2-week and 4-week culture response is observed to produce a proportional increase in T0 plant regeneration relative to the number of T0 plants regenerated in the control treatment where the starting seedlings receive no fipexide pre-treatment. The results in T0 plant regeneration is similar to those observed in Table 26 for the fipexide seedling pre-treatments.

Table 26 shows the results of the fipexide pre-treatment experiments. PTRT=pre-treatment, TRT=treatment.

TABLE 26

| | | | | T- | | | | |
|---|---|---|---|---|---|---|---|---|
| | PTRT | | | DNA | # culture | # culture | | |
| Seedling | conc | # | | score | response | response | # T0 | |
| PTRT | (mg/l) | Seedling | TRT | (0-5) | (2 wk) | (4 wk) | plants | TXN % |
| Fipexide | 0 | 42 | 1 | 4 | 590 | 607 | n/a | n/a |
| Fipexide | 4.75 | 42 | 2 | 4 | 768 | 829 | n/a | n/a |
| Fipexide | 9.5 | 42 | 3 | 3.5 | 681 | 780 | n/a | n/a |
| Fipexide | 19 | 42 | 4 | 3.5 | 636 | 733 | n/a | n/a |

### Example 12: Sugar Pre-Treatment Prior to Agrobacterium Infection Improved Transformation

Maize leaf segments/tissue were pre-treated with maltose or a combination of sugars, maltose and sucrose, prior to Agrobacterium infection to determine their effects on transformation success. In this example, maltose was used as a carbon source in place of sucrose.

Methods for Agrobacterium-mediated transformation of maize leaf segments/tissue were followed as outlined in Examples 4 and 5. Two replicates were conducted, one with Pioneer inbred ED85E and the second with Pioneer inbred GR013D. Specifically, seed of the inbreds was surface sterilized and sown on 90AE germination medium containing 0 mg/l maltose, 20 mg/l maltose, or 10 mg/l maltose+10 mg/l sucrose. 90AE medium contained 900 medium+2 mg/l ancymidol. The germination and growth period under 120 μmol m-2 s-1 light intensity using an 18-hour photoperiod at 28° C. was 13 to 16 days for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 (SEQ ID NO:4) plus PHP104215 (SEQ ID NO:352).

Pre-treating seedlings with maltose had a positive impact on maize leaf transformation (Table 27). Use of 20 mg/l maltose as the carbon source during seedling pre-treatments in inbred GR013D also improved T0 Txn % by 1.3-fold relative to results obtained after growing seedlings on 20 mg/l sucrose, which is the standard amount of sucrose in the 900 germination medium (see Table 14).

Table 27 shows the results of the maltose pre-treatment experiments. PTRT=pre-treatment, TRT=treatment.

TABLE 27

| | PTRT | | | T-DNA | # culture | # culture | | |
|---|---|---|---|---|---|---|---|---|
| Seedling | conc | # | | score | response | response | # T0 | |
| PTRT | (mg/l) | Seedling | TRT | (0-5) | (2 wk) | (4 wk) | plants | TXN % |
| Maltose | 0 | 30 | 1 | 3.5 | 203 | 202 | 142 | 473% |
| Maltose | 20 | 30 | 2 | 3.5 | 131 | 143 | 97 | 323% |
| Maltose + sucrose | 10 + 10 | 30 | 3 | 3.5 | 141 | 160 | 104 | 347% |
| Maltose | 0 | 30 | 1 | 4 | 601 | 526 | 19 | 63% |
| Maltose | 20 | 30 | 2 | 4 | 650 | 562 | 25 | 83% |
| Maltose + sucrose | 10 + 10 | 30 | 3 | 4 | 685 | 580 | 9 | 30% |

### Example 13: Pre-Treatment with Inhibitor of Abscisic Acid Biosynthesis Prior to Agrobacterium Infection Improved Transformation

Maize leaf segments/tissue were pre-treated with an inhibitor of abscisic acid biosynthesis, norflurazon, prior to Agrobacterium infection to determine its effect on transformation success.

Methods for Agrobacterium-mediated transformation of maize leaf segments/tissue were followed as outlined in Examples 4 and 5. Specifically, seed of the Pioneer inbred GR0112 was surface sterilized and sown on 90AE germination medium containing 0 mg/1, 0.1 mg/l, 1 mg/l, or 3 mg/l norflurazon. 90AE medium contained 900 medium+2 mg/l ancymidol. The germination and growth period under 120 μmol m-2 s-1 light intensity using an 18-hour photoperiod at 28° C. was 13 to 16 days for seedlings used in all replicate experiments and treatments.

Seedling leaf whorl tissue was isolated and mechanically processed to produce 0.5-3 mm leaf segments for transformation as described, using Agrobacterium strain LBA4404 TD THY-harboring PHP71539 (SEQ ID NO:4) plus PHP104222 (SEQ ID NO:353).

Pre-treating seedlings with norflurazon had a positive impact on maize leaf transformation (Table 28). Use of 0.1 mg/l norflurazon during seedling pre-treatments in inbred GR0112 improved T0 Txn % by 1.1-fold relative to the control treatment where the seedling germination medium contains no norflurazon within the first two weeks of culture after Agrobacterium infection.

Scoring the 4-week response and regenerating T0 plants is performed. The higher 2-week somatic embryo response for the 0.1 mg/l norflurazon seedling pretreatment is observed to produce a proportional increase in the 4-week somatic embryo response and is observed to produce a proportional increase in T0 plant regeneration relative to the control treatment where the starting seedlings receive no norflurazon. The results in 4-week somatic embryo response and T0 plant regeneration is similar to those observed in Table 28 for the norflurazon seedling pre-treatments.

Table 28 shows the results of the norflurazon pre-treatment experiments. PTRT=pre-treatment, TRT=treatment.

TABLE 28

| | PTRT | | | T-DNA | # culture | # culture | | |
|---|---|---|---|---|---|---|---|---|
| Seedling | conc | # | | score | response | response | # T0 | |
| PTRT | (mg/l) | Seedling | TRT | (0-5) | (2 wk) | (4 wk) | plants | TXN % |
| Norflurazon | 0 | 22 | 1 | 4 | 312 | n/a | n/a | n/a |
| Norflurazon | 0.1 | 22 | 2 | 4 | 350 | n/a | n/a | n/a |
| Norflurazon | 1 | 7 | 3 | 1.5 | 19 | n/a | n/a | n/a |
| Norflurazon | 3 | 7 | 4 | 2 | 45 | n/a | n/a | n/a |

When compared to control seedlings, seedlings pre-treated with an inhibitor of abscisic acid biosynthesis, including but not limited to inhibitors of abscisic acid biosynthesis listed in Table 23 (norflurazon, fluridone, diflufenican, abamine, nordihydroguaiaretic acid (NDGA)), are observed to produce leaf segments that exhibit an improvement in frequencies of T-DNA delivery, somatic embryo response (more rapid growth and higher numbers), production of TO plants, and single-copy integration frequencies.

### Example 14: Exposure of Seedlings to High Temperature Prior to Agrobacterium Infection Improved Transformation

### A. Heat Pre-Treatment of Seedlings Results in Increased Recovery of Transgenic T0 Plants

Methods for Agrobacterium-mediated transformation of maize leaf tissue were followed as outlined in Examples 4 and 5. Specifically, seed of Pioneer inbred PHH5E was surface sterilized and sown on germination medium containing 2 mg/l ancymidol (medium 70AE) with a 14-day growth period under 120 μmol m-2 s-1 light intensity using an 18-hour photoperiod at 28° C. At this point, the seedlings were divided into two treatments; 1) either remaining at 28° C. for an additional 3 hours, or 2) incubated at 45° C. for 3 hours, at which time all seedlings were mechanically processed in the presence of Agrobacterium suspension to produce suspended leaf segments/pieces for transformation. Seedling leaf whorl tissue was isolated and mechanically processed to produce 0.5-3 mm leaf segments for transformation, using Agrobacterium strain LBA4404 THY-TN-harboring PHP71539 plus PHP97334 (SEQ ID NO: 4 and 77, respectively).

As shown for paired treatments in five separate experiment (Table 29), transformation frequency for the seedlings pre-treated at 45° C. exhibited a higher Agrobacterium T-DNA delivery score (compared to the control) and produced approximately twice as many T0 plants as in the control treatment. This resulted in an overall mean and standard deviation for the control and 45° C. heat treatments of 260% (+/−113%) and 560% (+/−95%), respectively, which is a statistically significant increase (p=0.05).

TABLE 29

| Expt. | Seedling | No. | T-DNA | No. T0 | |
|---|---|---|---|---|---|
| No. | Treatment | Seedling | Delivery | plants | Txn % |
| 1 | Control | 25 | 2 | 61 | 244% |
| | 45° C. 3 hr* | 25 | 3 | 138 | 552% |
| 2 | Control | 25 | 2 | 48 | 192% |
| | 45° C. 3 hr | 23 | 3 | 112 | 487% |
| 3 | Control | 25 | 2 | 111 | 444% |
| | 45° C. 3 hr | 22 | 3 | 159 | 723% |
| 4 | Control | 23 | 2 | 34 | 148% |
| | 45° C. 3 hr | 25 | 3 | 134 | 536% |
| 5 | Control | 25 | 2 | 68 | 272% |
| | 45° C. 3 hr | 18 | 3 | 90 | 500% |

*seedlings incubated at 45° C., 70% relative humidity for 3 hours

### B. Milder Heat Pre-Treatment of Seedlings for an Extended Period of Time Results in a Consistently High Recovery of Transgenic T0 Plants

Seed of Pioneer inbred PHH5E was surface sterilized and sown on germination medium containing 2 mg/l ancymidol (medium 70AE) with a 14-day growth period under 120 μmol m-2 s-1 light intensity using an 18-hour photoperiod at 28° C. Seedling leaf whorl tissue was isolated and mechanically processed to produce 0.5-3 mm leaf segments for transformation as described, using Agrobacterium strain LBA4404 TD THY-harboring PHP71539 and PHP97334 (SEQ ID NO:4 and SEQ ID NO:77, respectively).

Prior to seedling harvesting, processing to produce leaf segments and Agrobacterium infection, seedlings were transferred into a 37° C., 50% relative humidity incubator for 17 hours. All seedlings were then dissected and mechanically processed to produce leaf segments for Agrobacterium transformation as described.

As shown in Table 30, transformation frequency for the seedlings pre-treated at 37° C. overnight exhibited a consistently high transformation frequency with a mean Txn % of 315% and a high single-copy frequency of 54% (mean value for 4 experiments).

TABLE 30

| Expt. | No. | No. T0 | | No. SC | |
|---|---|---|---|---|---|
| No. | Seedling | plants | Txn %* | T0s | SC %** |
| 1 | 10 | 33 | 330% | 21 | 64% |
| 2 | 10 | 19 | 190% | 9 | 47% |
| 3 | 10 | 32 | 320% | 14 | 44% |
| 4 | 10 | 42 | 420% | 24 | 57% |
| Average | 40 | 126 | 315% | 68 | 54% |

*Txn % = (Number of T0 plants/Starting seedlings) × 100
**SC % = (Number of T0 plants with a single-copy T-DNA/Starting seedlings) × 100

### C. Heat Pre-Treatment of Seedlings Results in Increased T-DNA Delivery, Increased Somatic Embryo Formation, Increased Recovery of Transgenic T0 Plants and Increased HDR Frequencies

Agrobacterium strain LBA4404 TD THY-harboring both PHP71539 (SEQ ID NO:4; the super-virulence plasmid) and PHP99721 (the T-DNA plasmid) was used for leaf transformation. The T-DNA of PHP99721 (SEQ ID NO:240) contained the components RB+LOXP+NOS::WUS2::IN2 TERM+3×ENH::UBI1ZM PRO::ODP2::OS-T28 TERM+HSP17.7 PRO::MO-CRE::PINII TERM+UBI1ZM PRO::CAS9::ZM-UBI TERM+ZM-U6 PRO::gRNA-CHR1-53.66+ZM-ALS PRO::HRA::SB-UBI TERM+CHR1-53.66 TARGET SITE+HOMOLOGY SEQ1+SI-UBI PRO::NPTII::SI-UBI TERM+HOMOLOGY SEQ2+CHR1-53.66 TARGET SITE+SB-UBI PRO::ZS-GREEN1::OS-UBI TERM+LB.

Seed of maize inbred PHH5E were surface sterilized and pressed lightly into solid germination medium (90AE) with the embryo axis-side upward, with subsequent germination and seedling growth occurring under light (120 μE m-2 s-1) using an 18-hour photoperiod at 28° C. for 14 days. On the morning the seedlings were to be used for transformation, half the seedlings were allowed to remain at 28° C. (Control Treatment) while the remaining half of the seedlings were transferred into an incubator at 45° C., 70% relative humidity for 3 hours (Heat Treatment). All the seedlings were then used to prepare leaf explants for transformation as described below.

First, the seedlings were cut above the mesocotyl (removing the aerial portions from the roots) and the first 3 cm of leaf whorl was harvested, discarding the remainder of the more mature leaf tissue. The 3-cm long leaf whorl was bisected longitudinally using a scalpel, and the halves were put into 100 ml of Agrobacterium suspension (OD=0.5-0.6 measured at 550 nm, with the bacterium suspended in medium 700J+200 mM AS+0.02% surfactant solution (Break-Thru-233®) in a food processor. The leaf tissue was pulse-blended on low speed (10 pulses) until the average size of leaf segments/fragments were approximately 0.5-3 mm in length/depth. The suspended segments/tissue in the Agrobacterium suspension remained in the blender bowl for 20 minutes at room temperature with gentle swirling every 1-2 minutes, which constituted the “Agrobacterium Infection” step. After infection, the suspension was poured through a sterile stainless-steel screen, catching the leaf fragments/segments from the liquid that passed through for disposal. The leaf segments were then transferred from the screen onto three layers of dry Whatman's #2 filter papers which wicked away excess Agrobacterium suspension (but not being washed) so that a thin layer of bacterium remained on the surface of the leaf segments/pieces. The leaf segments/pieces were again transferred onto a single layer of Whatman's filter paper resting on solid co-cultivation medium (710N) and were then cultured in the dark at 21° C. for 24 hours. After co-cultivation, the filter papers with the supported leaf segments/pieces were transferred onto resting medium 605B and cultured in the dark at 28° C. for one week, at which point the filter papers were again transferred onto selection medium 13266N and cultured in the dark at 28° C. for 3 weeks. After selection, the selection plated (held in a translucent culture box, typically holding 12 plates in 6 stacks of 2 plates) was transferred into a 45° C., 70% relative humidity incubator for two hours, then removed and the box placed on a benchtop at 25° C. for 1.5 hours for the temperature to re-equilibrate to room temperature. After heat treatment (which activated HSP17.7 PRO::CRE expression for excision of WUS/BBM/CRE from the T-DNA) healthy somatic embryos were transferred from the subtending filter papers onto fresh maturation medium 13329B and cultured for 2 weeks at 28° C. in the dark, then the plates were transferred into the light (120 μE m-2 s-1, 18-hour photoperiod) at 25° C. for one additional week. Healthy mature somatic embryos that had begun producing shoots were then transferred onto rooting medium 404J for an additional 203 weeks of culture under lights. Plantlets were then transferred to soil in the greenhouse. When regenerated T0 plants were large enough for sampling, leaf tissue was punched for qPCR analysis for T-DNA and Agrobacterium plasmid backbone sequences. PCR analysis for both HR junctions, and long-PCR analysis that spanned from the flanking endogenous Chromosome 1 sequences across the entire sequence that had integrated via homology-dependent repair (SDN3) were used to confirm targeted integration.

A total of 9 repeat experiments were carried out and for each transformation experiment, using 15-30 seedlings for either the control or the “heat-pre-treated seedling” treatments for each experiment. For gene editing designed specifically for gene insertion, the same construct PHP99721 was used.

The relative efficiency of T-DNA delivery was assessed by scoring transient expression of ZS-GREEN in leaf segments 3-4 days after Agrobacterium infection. Scores ranged from “0” in which no leaf segments/pieces within a given treatment expressed ZS-GREEN, with scores of 1, 2, 3, or 4, being used when approximately 25%, 50%, 75%, or 90-100% of the leaf segments/pieces within a treatment showed ZS-GREEN expression, respectively. Thus, we used transient expression of the visual marker as a relative indication of the efficiency of Agrobacterium T-DNA delivery. Using this scale, for all 9 experiments the T-DNA delivery score for the control treatments was consistently rated as “3” while for the heat treatment the score was consistently rated as “4”. Based on this observation, it was concluded that heat pretreatment of seedlings in an incubator at 45° C., 70% relative humidity for 3 hours prior to leaf segmentation and Agrobacterium infection resulted in increased efficiency of T-DNA delivery.

After T0 plants were produced, leaf samples were collected for PCR analysis to identify gene insertion events with NPTII gene. From 204 seedlings used in 9 completed transformation experiments, a total of 1150 T0 plants were produced, which gave an overall T0 transformation frequency of 563% (based on number of seedlings used for transformation). From the 1150 TO plants, 32 gene insertion events were confirmed using PCR that spanned each of the two-integration junctions and a long-PCR reaction that spanned the entire integration locus (both confirming correct respective insertion sizes), which yielded a 2.8% gene insertion frequency. Highly efficient HDR frequencies were observed for both the control and the “heat-pre-treated seedling” treatments. Heat shock treatment doubled the T0 transformation frequency and the gene editing (gene insertion) frequency in the TO population, thus heat shock treatment increased the overall process efficiency of gene editing (see Table 31).

TABLE 31

| | | | | | | | HDR % |
|---|---|---|---|---|---|---|---|
| Expt. | Seedling Pre- | No. | No. T0 | | HDR | T0 | by |
| No. | Treat | Seedling | Plants | Txn % | Confirm | HDR % | seedling |
| 1 | Control | 25 | 61 | 244% | 2 | 7.7 | 8 |
| 1 | Heat Trt. | 25 | 138 | 552% | 2 | 3.4 | 8 |
| 2 | Control | 25 | 48 | 192% | 0 | 0 | 0 |
| 2 | Heat Trt. | 23 | 112 | 487% | 3 | 5.6 | 13 |
| 3 | Control | 25 | 111 | 444% | 4 | 7.4 | 16 |
| 3 | Heat Trt. | 22 | 159 | 723% | 6 | 7.8 | 27.3 |
| 4 | Control | 23 | 34 | 148% | 0 | 0 | 0 |
| 4 | Heat Trt. | 25 | 134 | 536% | 7 | 11.5 | 28 |
| 5 | Control | 25 | 68 | 272% | 3 | 12.5 | 12 |
| 5 | Heat Trt. | 18 | 90 | 500% | 0 | 0 | 0 |
| 6 | Control | 25 | 59 | 236% | 2 | 5.7 | 8 |
| 6 | Heat Trt. | 15 | 114 | 760% | 5 | 10.4 | 33.3 |
| 7 | Control | 25 | 57 | 228% | 0 | 0 | 0 |
| 7 | Heat Trt. | 22 | 134 | 609% | 4 | 6.6 | 18.2 |
| 8 | Control | 25 | 65 | 260% | 4 | 11.8 | 16 |
| 8 | Heat Trt. | 22 | 136 | 618% | 3 | 4.1 | 13.6 |
| 9 | Control | 25 | 54 | 216% | 3 | 9.4 | 12 |
| 9 | Heat Trt. | 32 | 133 | 415% | 2 | 3.5 | 6.3 |
| Sum | Control | 223 | 557 | 250% | 18 | 3.2 | 8.1 |
| Sum | Heat Trt. | 204 | 1150 | 564% | 32 | 2.8 | 15.7 |

### D. Agrobacterium-Mediated Site-Specific Integration (SSI) in Heat Pre-Treated Seedling-Derived Leaf Segments/Tissue of Maize Inbreds

In a second pair of experiments, a pre-integrated target site (target locus) in the maize inbred PHH5E genome was used for site-specific integration, as described in U.S. Pat. Nos. 6,187,994, 6,262,341, 6,330,545, 6,331,661, and 8,586,361, each of which is herein incorporated by reference in its entirety. In this example, target site 10 located on chromosome 1 at genetic position 54.56 cM within the PHH5E inbred genome was used and is comprised of the integrated components loxP+UBI1ZM PRO::UBI1ZM 5′UTR::UBI1ZM INTRON1::FRT1::NPTII::PINII TERM+FRT6 (designated as the genomic Landing Pad) which had been previously introduced via Cas9-mediated homologous recombination to create this SSI landing pad. Seed of the PHH5E containing the genomic Landing Pad 10 were surface sterilized and germinated on 90AE medium for 14 days.

Seedling-derived leaf segments/pieces were transformed using the Agrobacterium strain LBA4404 TD THY-containing the helper plasmid PHP71539 (SEQ ID NO:4) in addition to the T-DNA-containing plasmid PHP90842 (T-DNA with RB+UBI::FLP+FRT1::PMI+ACTIN::WUS+UBI::ODP2+HSP17.7::CRE+LOXP+SB-UBI::DsRED2+FRT6+LB) and the second strain also contained PHP100712 (SEQ ID NO:342) (T-DNA with RB+LOXP+UBI1ZM PRO::UBI1ZM 5UTR::UBI1ZM INTRON1::WUS2::IN2 TERM+FMV ENHANCER+PCSV ENH+MMV ENH+SI-UBI1 PRO::SI-UBI1 INTRON1::ZM-ODP2::ZM-UBI TERM TERM+LOXP+NOS PRO::CRC::SB-GKAF TERM+LB) at a ratio of 9:1 (90842:100712). As shown in Table 32, seedlings pre-treated with an overnight 37° C. exposure produced substantially higher production of early somatic embryos (SE response) and a higher number of recovered Perfect Site-specific Integration events (RMCE events) when compared to the results for seedlings that were maintained at 28° C. during the same overnight period prior to Agrobacterium transformation.

TABLE 32

| | | Number of: | | | | | |
|---|---|---|---|---|---|---|---|
| Trt. | | SE response | Callus | T0 plant | RMCE | SSI | Escape |
| (° C.) | Seedlings | (%)** | event % | freq. (%) | events | events | % |
| 28 | 63 | 849 | 259 | 29 | 0 | 3 | 22 |
| 37 | 85 | 2041 | 141 | 22 | 3 | 1 | 83 |

Additional temperature ranges and durations for heat pre-treatment is observed to positively impact T-DNA delivery, somatic embryo formation, recovery of transgenic T0 plants, and HDR frequencies.

### Example 15: Auxin Pre-Treatment Prior to Agrobacterium Infection Improved Transformation

Methods for Agrobacterium-mediated transformation of maize leaf tissue were followed as outlined in Examples 4 and 5. Specifically, seed of Pioneer inbred PHH5E was surface sterilized and sown on germination medium containing no auxin for 14 days. The seedlings were then divided into four treatments: 1) remained on 900 medium (0 mg/l 2,4-D=control), 2) transferred onto 900 medium plus 3 mg/l 2,4-D, 3) transferred onto 900 medium plus 10 mg/l 2,4-D, or 4) transferred onto 900 medium plus 30 mg/l 2,4-D mg/l. All seedlings remained on these media for 24 hours under 120 μmol m-2 s-1 light intensity using an 18-hour photoperiod at 28° C., at which time all seedlings were mechanically processed in the presence of Agrobacterium suspension to produce suspended leaf segments/pieces for transformation.

Seedling leaf whorl tissue was isolated and mechanically processed to produce 0.5-3 mm leaf segments for transformation as described, using Agrobacterium strain LBA4404 TD THY-harboring PHP71539 and PHP97334 (SEQ ID NO:4 and SEQ ID NO:77).

Table 33 shows that growing seedlings on 10 mg/l 2,4-D resulted in improved leaf transformation, as demonstrated through both an increased transformation frequency (Txn %) and frequency of single-copy T-DNA integrations compared to the control treatment.

TABLE 33

| 2,4-D | | T-DNA | No. T0 | | No. SC | |
|---|---|---|---|---|---|---|
| (mg/l) | No. Seedlings | Delivery | Plants | Txn %* | Clean** | SC %*** |
| 0 | 60 | 3 | 55 | 92% | 27 | 45% |
| 3 | 60 | 3 | 45 | 75% | 18 | 30% |
| 10 | 60 | 4 | 72 | 120% | 40 | 67% |
| 30 | 60 | 4 | 28 | 47% | 19 | 32% |

*Transformation frequency = (No. T0 plants/No. Seedlings) × 100
**Single-copy T-DNA integration with no extraneous Agrobacterium sequences
***Single-copy Frequency = (No. Single-copy T0 plants/No. T0 plants) × 100

When compared to control seedlings, seedlings pre-treated with other auxins including, but not limited to auxins listed in Table 23 (2,4-D, dicamba, 2,4,5-T, NAA, picloram, indole-3-acetic acid, 4-chloroindole-3-acetic acid, phenylacetic acid, indole-3-butyric acid, and indole-3-propionic acid) is observed to produce leaf segments that exhibit an improvement in frequencies of T-DNA delivery, somatic embryo response (more rapid growth and higher numbers), production of T0 plants, and single-copy integration frequencies.

### Example 16: Anti-Oxidant Pre-Treatment Prior to Agrobacterium Infection to Improve Transformation

Methods for Agrobacterium-mediated transformation of maize leaf tissue are followed as outlined in Examples 4 and 5. Specifically, seed of Pioneer inbred PHH5E is surface sterilized and sown on germination medium containing no anti-oxidant for 14 days. At this point, the seedlings are divided into four treatments; 1) remaining on 900 medium (0 mg/l anti-oxidant=control), 2) being transferred onto 900 medium plus 3 mg/l anti-oxidant, 3) being transferred onto 900 medium plus 10 mg/l anti-oxidant, or 4) being transferred onto 900 medium plus 30 mg/l anti-oxidant. All seedlings remain on these media for 24 hours under 120 μmol m-2 s-1 light intensity using an 18-hour photoperiod at 28° C., at which time all seedlings are mechanically processed in the presence of Agrobacterium suspension to produce suspended leaf segments/pieces for transformation.

Seedling leaf whorl tissue is isolated and mechanically processed to produce 0.5-3 mm leaf segments for transformation as described, using Agrobacterium strain LBA4404 TD THY-harboring PHP71539 plus PHP97334 (SEQ ID NO:4 and SEQ ID NO:77).

When compared to control seedlings, seedlings pre-treated with an anti-oxidant, including but not limited to anti-oxidants listed in Table 23 (dithiolthreitol (DTT), cysteine, and glutathione), is observed to produce leaf segments that exhibit an improvement in frequencies of T-DNA delivery, somatic embryo response (more rapid growth and higher numbers), production of T0 plants, and single-copy integration frequencies.

### Example 17: Germination and Growth of Seedlings Under Increased-Spectrum Light Prior to Agrobacterium Infection Improved Transformation

Methods for Agrobacterium-mediated transformation of maize leaf tissue were followed as outlined in Examples 4 and 5. Specifically, seed of Pioneer inbred PHH5E was surface sterilized and sown on germination medium containing no auxin for 14 days, being grown under 120 μmol m-2 s-1 light intensity using an 18-hour photoperiod at 28° C. While light intensity remained consistent between treatments, the quality of the light was varied by growing seedlings under either fluorescent light (Phillips High-Performance Alto II, #F32T8/Plant), Valoya LED lights (Valoya NS12/C65 #LE17051487), or RAZR LED lights (Fluence Bioengineering, Inc. #4009716). The differences between these light sources were readily apparent when the output across the visible light spectrum was compared. The Phillips fluorescent lamp produced its broadest peak in the blue range (400-500 nm) with numerous sharp spikes and intervening gaps of weak illumination in the green, yellow, and red portions of the spectrum (500-700 nm). In comparison, the Razor LED array produced a sharp peak roughly in the middle of the blue (˜560-570 nm) with a broader peak extending across the green into the red (˜530-650 nm) portion of the spectrum, while the Valoya produced a sharp peak roughly in the middle of the blue (˜560-570 nm) with a broader peak across the green and yellow (˜530-630 nm) with a shoulder in the red (˜660-670 nm) portion of the spectrum.

Seedlings were transferred into an incubator at 37° C., 50% relative humidity for 24 hours before being mechanically processed. Seedling leaf whorl tissue was isolated and mechanically processed to produce 0.5-3 mm leaf segments for transformation as described, using Agrobacterium strain LBA4404 TD THY-harboring PHP71539 and PHP97334 (SEQ ID NO:4 and SEQ ID NO:77, respectively).

Table 34 shows that growing seedlings under different light spectra resulted in improved leaf transformation, as demonstrated through an increased transformation frequency (Txn %) under the RAZR LED lights, relative to those grown under either fluorescent or Valoya LED lighting.

TABLE 34

| | | | T-DNA | | |
|---|---|---|---|---|---|
| Expt | Light | No. | delivery | No. T0 | |
| No. | Source | Seedlings | score | Plants | Txn % |
| 1 | Fluorescent | 10 | 3 | 36 | 360% |
| | Valoya | 10 | 3 | 27 | 270% |
| | RAZR | 10 | 4 | 63 | 630% |
| 2 | Fluorescent | 10 | 3 | 35 | 350% |
| | Valoya | 10 | 3 | 29 | 290% |
| | RAZR | 10 | 4 | 52 | 520% |
| 3 | Fluorescent | 11 | 2 | 73 | 627% |
| | Valoya | 11 | 2 | 71 | 582% |
| | RAZR | 11 | 3 | 91 | 818% |
| Totals and | Fluorescent | 31 | 3 | 144 | x = 465% |
| mean % (x) | Valoya | 31 | 3 | 127 | x = 410% |
| | RAZR | 31 | 4 | 206 | x = 665% |

### Example 18: Soil-Sown Greenhouse-Grown Seedlings Under Full Sunlight Produce High Transformation Frequencies

Methods for Agrobacterium-mediated transformation of maize leaf tissue were followed as outlined in Examples 4 and 5. Specifically, seed of Pioneer inbred PHH5E was surface sterilized and sown in soil and grown under greenhouse conditions for 21 days. Harvested by cutting at soil level, brought into a sterile hood, sprayed with 70% ethanol, and then the outer three successive leaves were peeled back and removed, spraying and wiping with a 70% ethanol-soaked paper towel in between peeling off each leaf. Once the outer leaves were removed, the remaining inner leaf whorl was prepared as normal. The bottom 3 cm of surface-sterilized whorl was removed, bisected, and then mechanically processed in the presence of Agrobacterium suspension to produce suspended 0.5-3 mm leaf segments for transformation as described, using Agrobacterium strain LBA4404 TD THY-harboring PHP71539 plus PHP97334 (SEQ ID NO:4 and SEQ ID NO:77). When compared to seedlings grown under artificial lighting in growth chambers, seedling health under full-spectrum sunlight in the greenhouse is observed to be optimal. Further, seedlings grown under full-spectrum light in the greenhouse is observed to produce leaf segments that exhibit an improvement in frequencies of T-DNA delivery, somatic embryo response (more rapid growth and higher numbers), production of T0 plants, and single-copy integration frequencies.

It is contemplated that the leaf explant from a pre-treated seedling resulting from the methods disclosed herein can be derived from any plant found within the monocot families listed in Table 1 along with representative genera and/or species.

It is also expected that such additional treatments such as addition of ancymidol, 2,4-D, and either overnight or 3-hour heat treatment will have an additive effect, boosting transformation frequencies to even higher levels.

### Example 19: Use of Chlorine Gas for Seed Sterilization

Inbred PHH5E seed were placed in a monolayer within a sealed chamber that included a reservoir containing 100 ml of household bleach (8.25% (w/v) sodium hypochlorite) that was immediately below a stopcock valve in the top of the chamber. A glass pipette was used to add 3.5 ml of 12N HCL to the reaction container slowly through the open Valve-1 and the Valve-1 was immediately closed which sealed the chamber containing the seed. As the two solutions came into contact, chlorine gas was released from the reaction reservoir. The chamber remained closed to allow sterilization to proceed overnight (16-18 hrs). Two valves were then opened, Valve-2 was opened to allow chlorine gas to flow out of the seed-containing chamber and into a second scrubbing chamber containing 150 ml of 0.5M NaOH (that traps the chlorine) before the vented air was released into a chemical flow hood. Opening another Valve-3 in the seed-containing chamber allowed fresh air to flow into the chamber, allowing chlorine gas to evacuate and be replaced by fresh air. In this manner, the chamber was purged of chlorine gas for 1.5-2 hours before being opened to remove the seed.

The gas-sterilized seed were germinated on 90AE solid medium under (120 μE m-2 s-1) lights using an 18-hour photoperiod at 25° C. After 14 days on germination medium, the percentage of seed that germinated and the percentage exhibiting microbial contamination (fungal or bacterial) was evaluated. The results are shown in Table 35. Our standard aqueous sterilization method (described above) was also performed on the same batch of seed as a control (labeled as “Diluted Bleach” in Table 35).

TABLE 35

| | | | | # Seed | |
|---|---|---|---|---|---|
| Sterilization | # | # Germi- | % Germi- | Contam- | % Contam- |
| Method | Seed | nated | nated | inated | inated |
| Chlorine Gas | 50 | 43 | 86% | 30 | 60% |
| Chlorine Gas | 50 | 44 | 88% | 30 | 60% |
| Chlorine Gas | 50 | 46 | 92% | 15 | 30% |
| Chlorine Gas | 50 | 48 | 96% | 15 | 30% |
| Diluted Bleach | 50 | 48 | 96% | 0 | 0% |

The batch of PHH5E inbred seed used for this experiment typically resulted in 100% contamination if not sterilized before placing on the high-sucrose germination medium used in this experiment. As shown in Table 35, chlorine gas sterilization reduced contamination rates by 40% to 70%, and germination frequencies were in a similar range relative to the control treatment (aqueous diluted bleach sterilization). Noting that the aqueous bleach sterilization method is a product of careful parameter optimization (concentrations, time, temperature, etc), it is accordingly expected that optimization of parameters in the gas sterilization protocol will produce a similar highly efficient result.

### Example 20: Transformation of Maize Leaf Segments with ZM-ODP2 Alone

### A. Use of 3×ENH:UBI1ZM PRO

A plasmid containing the following T-DNA, RB+LOXP+FMV ENH::PSCV ENH::MMV ENH::UBI1ZM PRO::ZM-ODP2+HSP17.7 PRO::CRE+LOXP+SB-UBI::ZS-GREEN+SI-UBI::NPTII+LB, is constructed (PHV00001, SEQ ID NO: 343), where the 3×ENH:UBI1ZM PRO results in expression levels of ZM-ODP2 that are substantially higher than when using the UBI1ZM PRO alone.

When Agrobacterium strain LBA4404 TD THY—with PHP71539 (SEQ ID NO: 4) and a second plasmid PHV00001 (SEQ ID NO: 343) is used to transform maize inbred PH85E leaf segments, strongly expressed ZM-ODP2 is observed to result in rapid somatic embryo formation and stimulation of T0 plant generation.

The use of other viral or plant enhancer sequences, or EME sequences, such as those disclosed in WO2018/183878 which is incorporated herein by reference in its entirety, to add to the ZM-UBI promoter, or to substitute other strong promoters for ZM-UBI (homologous promoters from other species for example) along with enhancers or EMEs, is observed to produce similar results, with high levels of ZM-ODP2 expression, rapid somatic embryo formation, and generation of T0 plants.

### B. Use of a Two-Component Transactivation System

A plasmid containing the following T-DNA, RB+LOXP+ZM-GOS2 PRO::SB-UBI INTRON1::MO-LEXA:MO-CBF1A+6×REC:MIN35S PRO:OMEGA 5UTR::ZM-ODP2+HSP17.7 PRO::CRE+LOXP+SB-UBI:ZS-GREEN+SI-UBI::NPTII+LB, is constructed (PHV00003, SEQ ID NO: 345), where a two-component transactivation system results in expression levels of ZM-ODP2 that are substantially higher than when using UBI1ZM PRO::ODP2.

When Agrobacterium strain LBA4404 TD THY—with PHP71539 (SEQ ID NO: 4) and a second plasmid PHV0003 (SEQ ID NO: 345) is used to transform maize inbred PH85E leaf segments, it is observed that the strongly expressed ZM-ODP2 results in rapid somatic embryo formation and stimulation of T0 plant generation.

Modification to the components of the two-component transactivation system, such as (but not limited to) i) substituting a stronger promoter such as ZM-ACTIN PRO in place of ZM-GOS2, ii) substituting new activation domains in place of CBF1A, iii) altering the number of activation domains fused to the DNA binding domain, iv) and altering the number of LEXA-binding sequences (REC), is observed to further increase expression of ZM-ODP2. Substituting dCAS-alpha10 in place of LEXA and using gRNA sequences targeting the endogenous ZM-ODP2 promoter sequence is observed to stimulate ODP2 activity and thus promote rapid somatic embryos from transformed leaf cells.

### Example 21: Transformation of Maize Leaf Segments with ZM-WUS2 Alone

### A. Use of 3×ENH:UBI1ZM PRO

A plasmid containing the following T-DNA, RB+LOXP+FMV ENH::PSCV ENH::MMV ENH::UBI1ZM PRO::ZM-WUS2+HSP17.7 PRO::CRE+LOXP+SB-UBI::ZS-GREEN+SI-UBI::NPTII+LB, is constructed (PHV00002, SEQ ID NO: 344), where the 3×ENH:UBI1ZM PRO results in expression levels of ZM-WUS2 that are substantially higher than when using UBI1ZM PRO::WUS2.

When Agrobacterium strain LBA4404 TD THY—with PHP71539 (SEQ ID NO: 4) and a second plasmid PHV00002 (SEQ ID NO: 344) is used to transform maize inbred PH85E leaf segments, it is observed that the strongly expressed ZM-WUS2 results in rapid somatic embryo formation and stimulation of T0 plant generation.

The use of other viral or plant enhancer sequences, or EME sequences to add to the ZM-UBI promoter, or to substitute other strong promoters for ZM-UBI (homologous promoters from other species for example) along with enhancers or EMEs, produces similar results, with high levels of ZM-WUS2 expression, rapid somatic embryo formation, and generation of TO plants.

### B. Use of a Two-Component Transactivation System

A plasmid containing the following T-DNA, RB+LOXP+ZM-GOS2 PRO::SB-UBI INTRON1::MO-LEXA:MO-CBF1A+6×REC:MIN35S PRO:OMEGA 5UTR::ZM-WUS2+HSP17.7 PRO::CRE+LOXP+SB-UBI::ZS-GREEN+SI-UBI::NPTII+LB, is constructed (PHV00004, SEQ ID NO: 346), where a two-component transactivation system results in expression levels of ZM-WUS2 that are substantially higher than when using UBI1ZM PRO::WUS2.

When Agrobacterium strain LBA4404 TD THY—with PHP71539 (SEQ ID NO: 4) and a second plasmid PHV0004 (SEQ ID NO: 346) is used to transform maize inbred PH85E leaf segments, it is observed that the strongly expressed ZM-WUS2 results in rapid somatic embryo formation and stimulation of T0 plant generation.

Modification to the components of the two-component transactivation system, such as (but not limited to) i) substituting a stronger promoter such as ZM-ACTIN PRO in place of ZM-GOS2, ii) substituting new activation domains in place of CBF1A, iii) altering the number of activation domains fused to the DNA binding domain, iv) and altering the number of LEXA-binding sequences (REC), is used to further increase expression of ZM-WUS2. Substituting dCAS-alpha10 in place of LEXA and using gRNA sequences targeting the endogenous ZM-WUS2 promoter sequence stimulates WUS2 activity and thus promotes rapid somatic embryos from transformed leaf cells.

Modification to the components of the two-component transactivation system, such as (but not limited to) i) substituting a stronger promoter such as ZM-ACTIN PRO in place of ZM-GOS2, ii) substituting new activation domains in place of CBF1A, iii) altering the number of activation domains fused to the DNA binding domain, iv) and altering the number of LEXA-binding sequences (REC), is observed to further increase expression of ZM-WUS2. Substituting dCAS-alpha10 in place of LEXA and using gRNA sequences targeting the endogenous ZM-WUS2 promoter sequence is observed to stimulate WUS2 activity and thus promote rapid somatic embryos from transformed leaf cells.

### Example 22: Pre-Treatment of Seedling-Derived Leaf Pieces with Compounds that Inhibit PRC2 Improve Transformation Frequency

Methods for Agrobacterium-mediated transformation of maize leaf segments/tissue are followed as outlined in Examples 4 and 5. Specifically, seed of Pioneer inbred PHH5E is surface sterilized and sown on germination medium containing medium 900 medium plus 2 mg/l ancymidol. The germination and growth period under 120 μmol m-2 s-1 light intensity using an 18-hour photoperiod at 28° C. is 14 days for seedlings used in all replicate experiments and treatments.

Seedling leaf whorl tissue is isolated and mechanically processed to produce 0.5-3 mm leaf segments for 700J medium. The liquid suspension is then poured through a sterile metal sieve to catch the leaf pieces, and the leaf pieces are then transferred briefly to sterile dry filter papers to wick away excess moisture. The leaf pieces are then transferred into 700J medium plus a cocktail of PRC2 inhibitor compounds for 30 minutes. The PRC2 inhibitor cocktail can include the following chemicals: lirametostat (also called CPI-1205, CAS #1621862-70-1), tazemetostat (also called EPZ-6438, CAS #1403254-99-8), UNC1999 (CAS #1431612-23-5), CPI-169 (CAS #1450655-76-1), EPZ011989 (CAS #2095432-26-9), EP005687 (CAS #1396772-26-1), PF-067226304 (CAS #1616287-82-1), and JQ-EZ-05 (also referred to as JQEZ5, CAS #1913252-04-6). For the pretreatment, 700J medium containing 100 nM of a single PRC2 inhibitors, mixtures of two PRC2 inhibitors in the same solution (each at 100 nM), or mixtures of 3 or more PRC2 inhibitors can be used. After 30 minutes incubation in the PRC2 inhibitors, the leaf pieces are washed once with 700J medium, and then resuspended in 100 ml of Agrobacterium suspension in 700J medium plus acetosyringone for 20 minutes incubation at room temperature with periodic mild swirling of the suspension. After the 20 minutes Agrobacterium infection, using Agrobacterium strain LBA4404 TD THY which harbors PHP71539 and PHP97334 (SEQ ID NO: 4 and SEQ ID NO:77), the leaf pieces are removed from the Agrobacterium suspension, briefly dried on filter paper and then transferred onto filter papers on solid medium for one week of resting, three weeks of selection, maturation, and rooting as previously described in Examples 4 and 5.

A cocktail containing three PRC2 inhibitors that is used for the 30-minute pre-treatment of leaf pieces before Agrobacterium infection is, but is not limited to, 700J medium plus a mixture of 100 nM tazemetostat, 100 nM UNC1999, and 100 nM CPI-169. Exposure to this mixture of three PRC2 inhibitors, growth of transgenic somatic embryos is stimulated, and higher transformation frequencies are observed. A similar enhanced somatic embryogenesis and transformation frequency is observed when leaf pieces are pre-treated with a mixture containing lirameotostat, tazemetostat, and UNC1999 (each at 100 uM) for the 30 minutes. Since different plant varieties (such as maize inbreds) and different grass species respond differently to such pre-treatments, it is observed that different PRC2 inhibitor concentrations (for example between 0.01 nm and 10 uM) and durations of pre-treatment (for example, between 1 minute and 24 hours) are determined for each genotype to optimize somatic embryo formation and transformation frequencies.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

What is claimed is:

1. A method of pre-treating a seedling with at least one chemical compound, the method comprising contacting the seedling with the at least one chemical compound and isolating leaf explants from the seedling for transformation, wherein the transformation frequency of the leaf explants from the pre-treated seedling is higher than the transformation frequency of leaf explants from seedlings not treated with the at least one chemical compound.

2. The method of claim 1, wherein the at least one chemical compound is an auxin selected from the group consisting of 2,4-D, dicamba, 2,4,5-T, 1-Naphthaleneacetic acid (NAA), picloram, indole-3-acetic acid, 4-chloroindole-3-acetic acid, phenylacetic acid, indole-3-butyric acid, and indole-3-propionic acid.

3. The method of claim 1, wherein the at least one chemical compound is anti-oxidant selected from the group consisting of dithiolthreitol, cysteine, and glutathione.

4. The method of claim 1, wherein the at least one chemical compound is gibberellic acid inhibitors selected from the group consisting of ancymidol, paclobutrazol, uniconazole, chlormequat chloride, mepiquat, AMO-1618 [(2′-isopropyl-4′-(trimethylammoniumchloride)-5′-methylphenylpiperidinecarboxylate)], clorphonium-Cl, tetcylacis, flurprimidol, inabenfide, pro-hexadione, trinexapac-ethyl, daminozide, exo-16,17-acetate, malic hydrazide, phoshor D, alar, chlorocholine chloride, cycocel, the fungicides tebuconazole and metconazole, trinexapac-ethyl, prohexadione-calcium, and exo-16,17-dihydro-GA5-13-acetate.

5. The method of claim 1, wherein the at least one chemical compound is ancymidol.

6. The method of claim 5, wherein the seedling is contacted with ancymidol at a concentration of about 1 mg/l to about 2 mg/l.

7. The method of claim 1, wherein the at least one chemical compound is an inhibitor of abscisic acid biosynthesis selected from the group consisting of norflurazon, fluridone, diflufenican, abamine, and nordihydroguaiaretic acid (NDGA).

8. The method of claim 1, wherein the at least one chemical compound is an abscisic acid antagonist selected from the group consisting of AA1 [1-[(4-benzylpiperazin-1-yl)methyl]-2-sulfanylidene-1H,2H,5H,6H,7H,8H-[1,3,4]thiadiazolo[3,2-a][1,3]diazepin-5-one], AS6 [3′-hexylsulfanyl-ABA], DFPM ([5-(3,4-Dichlorophenyl) furan-2-yl]-piperidin-1-ylmethanethione), and RK460.

9. The method of claim 1, wherein the at least one chemical compound is the abscisic acid agonist, pyrabactin.

10. The method of claim 1, wherein the at least one chemical compound is a cytokinin selected from the group consisting of BAP [6-benylamino purine], BA [benzyladenine], kinetin, TDZ [thidiazuron], and trans-zeatin.

11. The method of claim 1, wherein the at least one chemical compound is an anti-cytokinin selected from the group consisting of S-4893 [a 4-phenylquinazoline derivative], 4-phenylquinazoline, 6-(2,5-Dihydroxybenzylamino) purine (LGR-991), and 2-hydroxy-3-methyl-benzyladenine.

12. The method of claim 1, wherein the at least one chemical compound is the cyclin-dependent kinase (CDK) inhibitor, caffeine.

13. The method of claim 1, wherein the at least one chemical compound is an auxin agonist selected from the group consisting of quinclorac, RubNeddins1 (RN1), RubNeddins2 (RN2), RubNeddins3 (RN3), and RubNeddins4 (RN4).

14. The method of claim 1, wherein the at least one chemical compound is an auxin antagonist selected from the group consisting of p-Chlorophenoxy isobutylic (PCIB), tert-butoxycarbonylaminohexyl-IAA (BH-Indole Acetic Acid=BH-IAA), α-(phenylethyl-2-oxo)-IAA (PEO-IAA), and α-(2,4-dimethylphenylethyl-2-oxo)-IAA (auxinole).

15. The method of claim 1, wherein the at least one chemical compound is fipexide.

16. The method of claim 1, wherein the at least one chemical compound is a methylation inhibitor selected from the group consisting of 5-azacytidine, Decitabine, RG108 [N-Phthalyl-L-tryptophan], zebularine, thioguanine, 2′-deoxy-5-fluoreocytidine, CM-272 (6-methoxy-2-(5-methyl-2-furyl)-N-(1-methyl-4-piperidyl)-7-(3-pyrrolidin-1-ylpropoxy) quinolin-4-amine), CM-579 (6-methoxy-2-(5-methyl-2-furyl)-N-[(1-methyl-4-piperidyl)methyl]-7-(3-pyrrolidin-1-ylpropoxy) quinolin-4-amine), SGI-1027 [N-[4-[(2-Amino-6-methyl-4-pyrimidinyl)amino]phenyl]-4-(4-quinolinylamino)benzamide], gamma-oryzanol, and (−)-epigallocatechin gallate (ECGC), β-Thujaplicin, and procainamide.

17. The method of claim 1, wherein the at least one chemical compound is a histone deacetylase (HDAC) inhibitor selected from the group consisting of valproic acid, tacedinaline, cambinol, trichostain A (TSA), verinostat, panobinostat, belinostat, RGFP-966 [(E)-N-(2-amino-4-fluorophenyl)-3-(1-cinnamyl-1H-pyrazol-4-yl) acrylamide], tubastatin A, and PCI-34051 [N-Hydroxy-1-[(4-methoxyphenyl)methyl]-1H-indole-6-carboxamide].

18. (canceled)

19. The method of claim 1, wherein the at least one chemical compound is a sugar composition selected from the group consisting of sucrose and maltose.

20. The method of claim 1, wherein the at least one chemical compound is a composition comprising a metal selected from the group consisting of copper, zinc, and manganese.

21. The method of claim 1, wherein the at least one chemical compound is an amino acid selected from the group consisting of proline, asparagine, methionine, and glutamine.

22. The method of claim 1, wherein the at least one chemical compound is an ethylene inhibitor selected from the group consisting of silver nitrate, 2-aminoethoxyvinyl glycine (AVG), silver ions (Ag), and 1-methylcyclopropene (1-MCP).

23. The method of claim 1, wherein the at least one chemical compound is the ethylene competitive inhibitor, norbornadiene.

24. The method of claim 1, wherein the at least one chemical compound is a Polycomb Repressive Complex 2 (PRC2) inhibitor selected from the group consisting of EPZ005687 [1-cyclopentyl-N-[(4,6-dimethyl-2-oxo-1H-pyridin-3-yl)methyl]-6-[4-(morpholin-4-ylmethyl)phenyl]indazole-4-carboxamide], UNC1999 [N-[(6-methyl-2-oxo-4-propyl-1H-pyridin-3-yl)methyl]-1-propan-2-yl-6-[6-(4-propan-2-ylpiperazin-1-yl) pyridin-3-yl]indazole-4-carboxamide], PF-06726304 (C₂₂H₂₁Cl₂N₃O₃), Lirametostat, Tazemetostat, EPZ011989 (C₃₅H₅₁N₅O₄·HCl), CPI-169 (C₂₇H₃₆N₄O₅S), and JQ-EZ-05 (C₃₀H₃₈N₈O₂).

25. The method of claim 1, wherein the at least one chemical compound is a Jasmonate, selected from the group consisting of Jasmonic acid and Methyl-jasmonate.

26. The method of claim 1, wherein the at least one chemical compound is a safener selected from the group consisting of naphthalic anhydride, salicylic acid, fenchlorazole, cloquintocet-mexyl, and mefenpyr diethyl.

27. The method of claim 1, wherein the at least one chemical compound is a kinase inhibitor selected from the group consisting of N-[(2R)-2,3 dihydroxypropoxy]-3,4-difluoro-2-(2-fluoro-4-iodoanilino) benzamide, anthra (1,9-cd) pyrazol-6 (2H)-one:4-(4-Fluoro phenyl)-2-(4-methylsulfinylphenyl)-5-(4-pyridyl) 1H imidazole, and N-benzyl-2-(pyrimidin-4-ylamino)-1,3 thiazole-4-carboxamide.

28. The method of claim 1, wherein the at least one chemical compound is a reactive oxygen species (ROS) scavenger selected from the group consisting of Tiron, dimethylthiourea (DMTU), and diphenyleneiodonium (DPI).

29. The method of claim 1, wherein the at least one chemical compound is 2,4-D, dicamba, 2,4,5-T, NAA, picloram, indole-3-acetic acid, 4-chloroindole-3-acetic acid, phenylacetic acid, indole-3-butyric acid, indole-3-propionic acid, dithiolthreitol (DTT), cysteine, glutathione, ancymidol, paclobutrazol, uniconazole, chlormequat chloride, mepiquat, AMO-1618 [(2′-isopropyl-4′-(trimethylammoniumchloride)-5′-methylphenylpiperidinecarboxylate)], clorphonium-Cl, tetcylacis, flurprimidol, inabenfide, pro-hexadione, trinexapac-ethyl, daminozide, exo-16,17-acetate, malic hydrazide, phoshor D, alar, chlorocholine chloride, cycocel, the fungicides tebuconazole and metconazole, trinexapac-ethyl, prohexadione-calcium, exo-16,17-dihydro-GA5-13-acetate, norflurazon, fluridone, diflufenican, abamine, nordihydroguaiaretic acid (NDGA), AA1 [1-[(4-benzylpiperazin-1-yl)methyl]-2-sulfanylidene-1H,2H,5H,6H,7H,8H-[1,3,4]thiadiazolo[3,2-a][1,3]diazepin-5-one], AS6 [3′-hexylsulfanyl-ABA], DFPM ([5-(3,4-Dichlorophenyl)furan-2-yl]-piperidin-1-ylmethanethione), RK460, pyrabactin, BAP [6-benylamino purine], BA [benzyladenine], kinetin, TDZ [thidiazuron], trans-zeatin, S-4893 [a 4-phenylquinazoline derivative], 4-phenylquinazoline, 6-(2,5-Dihydroxybenzylamino) purine (LGR-991), 2-hydroxy-3-Methyl-benzyladenine, caffeine, quinclorac, RubNeddins1 (RN1), RubNeddins2 (RN2), RubNeddins3 (RN3), RubNeddins4 (RN4), p-Chlorophenoxy isobutylic (PCIB), tert-butoxycarbonylaminohexyl-IAA (BH-Indole Acetic Acid=BH-IAA), α-(phenylethyl-2-oxo)-IAA (PEO-IAA), α-(2,4-dimethylphenylethyl-2-oxo)-IAA (auxinole), fipexide, 5-azacytidine, Decitabine, RG108 [N-Phthalyl-L-tryptophan], zebularine, thioguanine, 2′-deoxy-5-fluoreocytidine, CM-272 (6-methoxy-2-(5-methyl-2-furyl)-N-(1-methyl-4-piperidyl)-7-(3-pyrrolidin-1-ylpropoxy) quinolin-4-amine), CM-579 (6-methoxy-2-(5-methyl-2-furyl)-N-[(1-methyl-4-piperidyl)methyl]-7-(3-pyrrolidin-1-ylpropoxy) quinolin-4-amine), SGI-1027 [N-[4-[(2-Amino-6-methyl-4-pyrimidinyl)amino]phenyl]-4-(4-quinolinylamino)benzamide], gamma-oryzanol, and (−)-epigallocatechin gallate (ECGC), β-Thujaplicin, procainamide, valproic acid, tacedinaline, cambinol, trichostain A (TSA), verinostat, panobinostat, belinostat, RGFP-966 [(E)-N-(2-amino-4-fluorophenyl)-3-(1-cinnamyl-1H-pyrazol-4-yl) acrylamide], tubastatin A, PCI-34051 [N-Hydroxy-1-[(4-methoxyphenyl)methyl]-1H-indole-6-carboxamide], sucrose, maltose, copper, zinc, manganese, proline, asparagine, methionine, glutamine, silver nitrate, 2-aminoethoxyvinyl glycine (AVG), silver ions (Ag), 1-methylcyclopropene (1-MCP), norbornadiene, EPZ005687 [1-cyclopentyl-N-[(4,6-dimethyl-2-oxo-1H-pyridin-3-yl)methyl]-6-[4-(morpholin-4-ylmethyl)phenyl]indazole-4-carboxamide], UNC1999 [N-[(6-methyl-2-oxo-4-propyl-1H-pyridin-3-yl)methyl]-1-propan-2-yl-6-[6-(4-propan-2-ylpiperazin-1-yl) pyridin-3-yl]indazole-4-carboxamide], PF-06726304 [C₂₂H₂₁Cl₂N₃O₃], Lirametostat, Tazemetostat, EPZ011989 (C₃₅H₅₁N₅O₄·HCl), CPI-169 (C₂₇H₃₆N₄O₅S), and JQ-EZ-05 (C₃₀H₃₈N₈O₂), Jasmonic acid, Methyl-jasmonate, naphthalic anhydride, salicylic acid, fenchlorazole, cloquintocet-mexyl, mefenpyr diethyl, N-[(2R)-2,3 dihydroxypropoxy]-3,4-difluoro-2-(2-fluoro-4-iodoanilino) benzamide, anthra (1,9-cd) pyrazol-6 (2H)-one:4 (4-Fluoro phenyl)-2-(4-methylsulfinylphenyl)-5-(4-pyridyl) 1H imidazole, N-benzyl-2-(pyrimidin-4-ylamino)-1,3 thiazole-4-carboxamide, Tiron, dimethylthiourea (DMTU), diphenyleneiodonium (DPI), or any combinations thereof.

30. (canceled)

31. A method of pre-treating a seedling, comprising exposing the seedling to an increased spectrum of light, an increased heat treatment, or a cold-shock treatment, and isolating leaf explants from the exposed seedling for transformation, wherein the transformation frequency of the leaf explants from the pre-treated seedling is higher than the transformation frequency of leaf explants from an unexposed seedling.

32. The method of claim 31, wherein the heat treatment is 45° C. for 3 hours at 70% relative humidity.

33. The method of claim 31, wherein the heat treatment is 37° C. for 17 hours at 50% relative humidity.

34. (canceled)

35. The method of claim 1, wherein the seedling is further contacted with a non-chemical pre-treatment comprising exposing the seedling to an increased spectrum of light, an increased heat treatment, or a cold-shock treatment, and wherein the pre-treatment of the seedling with the at least one chemical compound and the non-chemical pre-treatment occur simultaneously or sequentially, and isolating leaf explants from the exposed seedling for transformation, wherein the transformation frequency of the leaf explants from the pre-treated seedling is higher than the transformation frequency of leaf explants from an unexposed seedling.

36. (canceled)
